SEQUENCE LISTING (78 sequences)

SEQ ID NO: 1 - protein, 10 aa, synthetic construct
GVYDGREHTV

SEQ ID NO: 2 - protein, 6 aa, synthetic construct
VSPFSN

SEQ ID NO: 3 - protein, 7 aa, synthetic construct
LTFSENT

SEQ ID NO: 4 - protein, 13 aa, synthetic construct
VVNSAQGLYIPTF

SEQ ID NO: 5 - protein, 113 aa, synthetic construct
ANQVEQSPQSLIILEGKNVTLQCQYTVSPFSNLRWYKQDTGRGPVSLTILTFSENTKSNGRYTATLDADTKQSSLHITASQLSDSASYICVVNSAQGLYIPTFGRGTSLIVHP

SEQ ID NO: 6 - protein, 113 aa, synthetic construct
ANQVEQSPQSLIILEGKQVTLQCQYTVSPFSNLRWYKQDTGRGPVSLTILTFSENTKSNGRYTATLDADTKQSSLHITASQLSDSASYICVVNSAQGLYIPTFGRGTSLIVHP

SEQ ID NO: 7 - protein, 113 aa, synthetic construct
ANQVEQSPQSLIILEGKNVTLQCNYTVSPFSNLRWYKQDTGRGPVSLTILTFSENTKSNGRYTATLDADTKQSSLHITASQLSDSASYICVVNSAQGLYIPTFGRGTSLIVHP

SEQ ID NO: 8 - protein, 5 aa, synthetic construct
LDHEN

SEQ ID NO: 9 - protein, 6 aa, synthetic construct
SRFATG

SEQ ID NO: 10 - protein, 16 aa, synthetic construct
ASSSDQNSGDPYEQYF

SEQ ID NO: 11 - protein, 116 aa, synthetic construct
DVKVTQSSRYLVKRTGEKVFLECVQDLDHENMFWYRQDPGLGLRLIYFSRFATGKEKGDIPEGYSVSREKKERFSLILESASTQQTSMYLCASSSDQNSGDPYEQYFGPGTRLTVT

SEQ ID NO: 12 - protein, 116 aa, synthetic construct
DVKVTQSSRYLVKRTGEKVFLECVQDLDHENMFWYRQDPGLGLRLIYFSRFATGKEKGDIPEGYSVSREKKERFSLILESASTNQTSMYLCASSSDQNSGDPYEQYFGPGTRLTVT

SEQ ID NO: 13 - protein, 26 aa, synthetic construct
ANQVEQSPQSLIILEGKNVTLQCQYT

SEQ ID NO: 14 - protein, 26 aa, synthetic construct
ANQVEQSPQSLIILEGKQVTLQCQYT

SEQ ID NO: 15 - protein, 26 aa, synthetic construct
ANQVEQSPQSLIILEGKNVTLQCNYT

SEQ ID NO: 16 - protein, 17 aa, synthetic construct
LRWYKQDTGRGPVSLTI

SEQ ID NO: 17 - protein, 34 aa, synthetic construct
KSNGRYTATLDADTKQSSLHITASQLSDSASYIC

SEQ ID NO: 18 - protein, 10 aa, synthetic construct
GRGTSLIVHP

SEQ ID NO: 19 - protein, 26 aa, synthetic construct
DVKVTQSSRYLVKRTGEKVFLECVQD

SEQ ID NO: 20 - protein, 17 aa, synthetic construct
MFWYRQDPGLGLRLIYF

SEQ ID NO: 21 - protein, 37 aa, synthetic construct
KEKGDIPEGYSVSREKKERFSLILESASTQQTSMYLC

SEQ ID NO: 22 - protein, 37 aa, synthetic construct
KEKGDIPEGYSVSREKKERFSLILESASTNQTSMYLC

SEQ ID NO: 23 - protein, 9 aa, synthetic construct
GPGTRLTVT

SEQ ID NO: 24 - protein, 199 aa, synthetic construct
ANQVEQSPQSLIILEGKNVTLQCQYTVSPFSNLRWYKQDTGRGPVSLTILTFSENTKSNGRYTATLDADTKQSSLHITASQLSDSASYICVVNSAQGLYIPTFGRGTSLIVHPYIQKPDPAVYQLRDSKSSDKSVCLFTDFDSQTQVSQSKDSDVYITDKCVLDMRSMDFKSNSAVAWSQKSDFACANAFQNSIIPEDT

SEQ ID NO: 25 - protein, 199 aa, synthetic construct
ANQVEQSPQSLIILEGKQVTLQCQYTVSPFSNLRWYKQDTGRGPVSLTILTFSENTKSNGRYTATLDADTKQSSLHITASQLSDSASYICVVNSAQGLYIPTFGRGTSLIVHPYIQKPDPAVYQLRDSKSSDKSVCLFTDFDSQTQVSQSKDSDVYITDKCVLDMRSMDFKSNSAVAWSQKSDFACANAFQNSIIPEDT

SEQ ID NO: 26 - protein, 199 aa, synthetic construct
ANQVEQSPQSLIILEGKNVTLQCNYTVSPFSNLRWYKQDTGRGPVSLTILTFSENTKSNGRYTATLDADTKQSSLHITASQLSDSASYICVVNSAQGLYIPTFGRGTSLIVHPYIQKPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 27 - protein, 246 aa, synthetic construct
DVKVTQSSRYLVKRTGEKVFLECVQDLDHENMFWYRQDPGLGLRLIYFSRFATGKEKGDIPEGYSVSREKKERFSLILESASTQQTSMYLCASSSDQNSGDPYEQYFGPGTRLTVTEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 28 - protein, 246 aa, synthetic construct
DVKVTQSSRYLVKRTGEKVFLECVQDLDHENMFWYRQDPGLGLRLIYFSRFATGKEKGDIPEGYSVSREKKERFSLILESASTNQTSMYLCASSSDQNSGDPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 29 - protein, 86 aa, synthetic construct
YIQKPDPAVYQLRDSKSSDKSVCLFTDFDSQTQVSQSKDSDVYITDKCVLDMRSMDFKSNSAVAWSQKSDFACANAFQNSIIPEDT

SEQ ID NO: 30 - protein, 86 aa, synthetic construct
YIQKPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 31 - protein, 130 aa, synthetic construct
EDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 32 - protein, 130 aa, synthetic construct
EDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 33 - protein, 10 aa, synthetic construct
DKTHTCPPCP

SEQ ID NO: 34 - protein, 5 aa, synthetic construct
GGGGS

SEQ ID NO: 35 - protein, 5 aa, synthetic construct
GGGSG

SEQ ID NO: 36 - protein, 5 aa, synthetic construct
GGSGG

SEQ ID NO: 37 - protein, 5 aa, synthetic construct
GSGGG

SEQ ID NO: 38 - protein, 6 aa, synthetic construct
GSGGGP

SEQ ID NO: 39 - protein, 5 aa, synthetic construct
GGEPS

SEQ ID NO: 40 - protein, 7 aa, synthetic construct
GGEGGGP

SEQ ID NO: 41 - protein, 12 aa, synthetic construct
GGEGGGSEGGGS

SEQ ID NO: 42 - protein, 8 aa, synthetic construct
GGGSGGGG

SEQ ID NO: 43 - protein, 20 aa, synthetic construct
GGGGSGGGGSGGGGSGGGGS

SEQ ID NO: 44 - protein, 5 aa, synthetic construct
EAAAK

SEQ ID NO: 45 - protein, 15 aa, synthetic construct
EAAAKEAAAKEAAAK

SEQ ID NO: 46 - protein, 24 aa, synthetic construct
GGGGSGGGGSGGGGSGGGGSGGGS

SEQ ID NO: 47 - protein, 15 aa, synthetic construct
EPKSSDKTHTCPPCP

SEQ ID NO: 48 - protein, 12 aa, synthetic construct
ESKYGPPCPSCP

SEQ ID NO: 49 - protein, 217 aa, synthetic construct
APELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 217 aa, synthetic construct
APELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 681 aa, synthetic construct
GACAAGACCCACACCTGTCCTCCATGTCCTGCTCCAGAACTGCTCGGCGGACCTTCCGTGTTCCTGTTTCCTCCAAAGCCTAAGGACACCCTGATGATCTCTCGGACCCCTGAAGTGACCTGCGTGGTGGTGGATGTGTCTCACGAGGATCCCGAAGTGAAGTTCAATTGGTACGTGGACGGCGTGGAAGTGCACAACGCCAAGACCAAGCCTAGAGAGGAACAGTACGGCTCCACCTACAGAGTGGTGTCCGTGCTGACAGTGCTGCACCAGGATTGGCTGAACGGCAAAGAGTACAAGTGCAAGGTGTCCAACAAGGCCCTGCCTGCCCCAATCGAAAAGACCATCTCCAAGGCCAAGGGCCAGCCTAGGGAACCCCAGGTTTACACCCTGCCTCCAAGCCGGGATGAGCTGACCAAGAACCAGGTGTCCCTGTGGTGCCTGGTCAAGGGCTTCTACCCTTCCGATATCGCCGTGGAATGGGAGAGCAATGGCCAGCCTGAGAACAACTACAAGACAACCCCTCCTGTGCTGGACTCCGACGGCTCATTCTTCCTGTACTCCAAGCTGACCGTGGACAAGTCCAGATGGCAGCAGGGCAACGTGTTCTCCTGCTCCGTGATGCACGAGGCCCTGCACAATCACTACACCCAGAAGTCCCTGTCTCTGTCCCCTGGCAAA

SEQ ID NO: 54 - protein, 426 aa, synthetic construct
ANQVEQSPQSLIILEGKQVTLQCQYTVSPFSNLRWYKQDTGRGPVSLTILTFSENTKSNGRYTATLDADTKQSSLHITASQLSDSASYICVVNSAQGLYIPTFGRGTSLIVHPYIQKPDPAVYQLRDSKSSDKSVCLFTDFDSQTQVSQSKDSDVYITDKCVLDMRSMDFKSNSAVAWSQKSDFACANAFQNSIIPEDTDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 55 - protein, 1277 aa, synthetic construct
CCAACCAGGTGGAACAGTCCCCTCAGAGCCTGATCATCCTGGAAGGCAAGCAGGTGACCCTGCAGTGCCAGTACACCGTGTCTCCCTTCTCCAACCTGCGGTGGTACAAGCAGGATACCGGCAGAGGACCTGTGTCTCTGACCATCCTGACCTTCTCCGAGAACACCAAGTCCAACGGCCGGTACACCGCCACACTGGATGCTGACACAAAGCAGTCCAGCCTGCACATCACCGCCTCTCAGCTGTCTGACTCCGCCTCCTACATCTGCGTGGTCAATTCTGCCCAGGGCCTGTACATCCCCACCTTCGGAAGAGGCACCAGCCTGATCGTGCACCCCTACATCCAGAAACCTGATCCTGCCGTGTACCAGCTGAGAGACAGCAAGTCCAGCGACAAGAGCGTGTGCCTGTTCACCGACTTCGACAGCCAGACCCAAGTGTCCCAGAGCAAGGACAGCGACGTGTACATCACCGATAAGTGCGTGCTGGACATGCGGAGCATGGACTTCAAGAGCAACAGCGCCGTGGCCTGGTCCCAAAAGAGCGATTTCGCCTGCGCCAACGCCTTCCAAAACAGCATTATCCCCGAGGACACAGACAAGACCCACACCTGTCCTCCATGTCCTGCTCCAGAACTGCTCGGCGGACCTTCCGTGTTCCTGTTTCCTCCAAAGCCTAAGGACACCCTGATGATCTCTCGGACCCCTGAAGTGACCTGCGTGGTGGTGGATGTGTCTCACGAGGATCCCGAAGTGAAGTTCAATTGGTACGTGGACGGCGTGGAAGTGCACAACGCCAAGACCAAGCCTAGAGAGGAACAGTACGGCTCCACCTACAGAGTGGTGTCCGTGCTGACAGTGCTGCACCAGGATTGGCTGAACGGCAAAGAGTACAAGTGCAAGGTGTCCAACAAGGCCCTGCCTGCCCCAATCGAAAAGACCATCTCCAAGGCCAAGGGCCAGCCTAGGGAACCCCAGGTTTACACCCTGCCTCCAAGCCGGGATGAGCTGACCAAGAACCAGGTGTCCCTGTCCTGCGCCGTCAAGGGCTTCTACCCTTCCGATATCGCCGTGGAATGGGAGAGCAATGGCCAGCCTGAGAACAACTACAAGACAACCCCTCCTGTGCTGGACTCCGACGGCTCATTCTTCCTGGTGTCCAAGCTGACCGTGGACAAGTCCAGATGGCAGCAGGGCAACGTGTTCTCCTGCTCCGTGATGCACGAGGCCCTGCACAATCACTACACCCAGAAGTCCCTGTCTCTGTCCCCTGGCAAA

SEQ ID NO: 56 - protein, 504 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSSGGGGSDVKVTQSSRYLVKRTGEKVFLECVQDLDHENMFWYRQDPGLGLRLIYFSRFATGKEKGDIPEGYSVSREKKERFSLILESASTQQTSMYLCASSSDQNSGDPYEQYFGPGTRLTVTEDLKNVFPPEVAVFEPSEAEISHTQKATLVCLATGFYPDHVELSWWVNGKEVHSGVCTDPQPLKEQPALQDSRYALSSRLRVSATFWQDPRNHFRCQVQFYGLSENDEWTQDRAKPVTQIVSAEAWGRAD

SEQ ID NO: 57 - protein, 1512 aa, synthetic construct
GCAATTCAGATGACTCAAAGTCCGAGTTCTTTGTCTGCGAGCGTTGGGGATCGTGTGACGATTACATGTCGCGCCAGCCAAGACATTCGCAATTATCTGAACTGGTATCAACAGAAGCCAGGGAAGGCGCCGAAACTGTTAATCTATTACACATCCCGCCTGGAATCGGGAGTTCCTTCGCGCTTTAGCGGCTCCGGCAGCGGCACTGATTACACGTTGACCATTAGCAGCTTACAGCCTGAAGATTTTGCCACTTATTATTGTCAGCAGGGAAACACTCTGCCATGGACCTTCGGGCAGGGCACGAAAGTGGAAATCAAGGGTGGTGGTGGCAGTGGTGGAGGCGGATCAGGGGGCGGGGGAAGCGGCGGAGGCGGTTCAGGTGGCGGAAGCGAGGTGCAGTTAGTGGAGAGTGGCGGCGGTTTGGTTCAACCAGGTGGTTCCTTGCGCCTGTCATGCGCGGCTTCAGGCTACTCATTCACCGGCTATGCCATGAATTGGGTGCGTCAGGCACCCGGTAAAGGTTTAGAATGGGTGGCGCTGATTAATCCGTATAAGGGGGTATCTACCTACAACCAGAAGTTCAAAGATCGCTTCACATTTTCTGTAGACAAATCTAAAAATACTGCCTACTTGCAGATGAATAGTCTGCGTGCAGAAGACACAGCAGTTTACTACTGCGCCCGCTCGGGTTACTACGGCGATTCAGACTGGTACTTTGATGTGTGGGGACAGGGAACGTTAGTAACAGTGTCCTCCGGTGGTGGCGGCTCCGACGTGAAAGTGACCCAGAGCAGCAGATACCTGGTCAAGAGAACCGGCGAGAAGGTGTTCCTGGAATGCGTGCAGGACCTGGACCACGAGAACATGTTTTGGTACAGACAGGACCCCGGCCTGGGCCTGAGACTGATCTATTTCTCCAGATTCGCCACCGGCAAAGAGAAGGGCGATATCCCCGAGGGCTACTCCGTGTCCAGAGAGAAGAAAGAGCGGTTCTCCCTGATCCTGGAAAGCGCCTCTACCCAGCAGACCTCTATGTACCTGTGCGCCTCCTCCAGCGACCAGAACTCTGGCGATCCTTACGAGCAGTACTTCGGCCCAGGCACCAGACTGACCGTGACAGAAGATCTGAAGAACGTGTTCCCACCTGAGGTGGCCGTGTTCGAGCCTTCTGAGGCCGAGATCAGCCACACCCAGAAAGCCACCCTCGTGTGTCTGGCCACAGGTTTCTACCCCGACCACGTGGAACTGTCTTGGTGGGTCAACGGCAAAGAGGTGCACTCCGGCGTGTGCACCGATCCCCAGCCCCTGAAAGAACAGCCCGCCCTGCAAGACAGCAGATACGCCCTGAGCAGCAGGCTGAGAGTGTCCGCCACCTTCTGGCAGGACCCCCGGAATCACTTCAGGTGCCAGGTGCAGTTCTACGGCCTGAGCGAGAACGACGAGTGGACCCAGGACAGAGCCAAGCCCGTGACCCAGATCGTGTCTGCCGAAGCCTGGGGCAGAGCTGAT

SEQ ID NO: 58 - protein, 426 aa, synthetic construct
ANQVEQSPQSLIILEGKNVTLQCQYTVSPFSNLRWYKQDTGRGPVSLTILTFSENTKSNGRYTATLDADTKQSSLHITASQLSDSASYICVVNSAQGLYIPTFGRGTSLIVHPYIQKPDPAVYQLRDSKSSDKSVCLFTDFDSQTQVSQSKDSDVYITDKCVLDMRSMDFKSNSAVAWSQKSDFACANAFQNSIIPEDTDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 59 - protein, 1278 aa, synthetic construct
GCCAACCAGGTGGAACAGTCCCCTCAGAGCCTGATCATCCTGGAAGGCAAGAACGTGACCCTGCAGTGCCAGTACACCGTGTCTCCCTTCTCCAACCTGCGGTGGTACAAGCAGGATACCGGCAGAGGACCTGTGTCTCTGACCATCCTGACCTTCTCCGAGAACACCAAGTCCAACGGCCGGTACACCGCCACACTGGATGCTGACACAAAGCAGTCCAGCCTGCACATCACCGCCTCTCAGCTGTCTGACTCCGCCTCCTACATCTGCGTGGTCAATTCTGCCCAGGGCCTGTACATCCCCACCTTCGGAAGAGGCACCAGCCTGATCGTGCACCCCTACATCCAGAAACCTGATCCTGCCGTGTACCAGCTGAGAGACAGCAAGTCCAGCGACAAGAGCGTGTGCCTGTTCACCGACTTCGACAGCCAGACCCAAGTGTCCCAGAGCAAGGACAGCGACGTGTACATCACCGATAAGTGCGTGCTGGACATGCGGAGCATGGACTTCAAGAGCAACAGCGCCGTGGCCTGGTCCCAAAAGAGCGATTTCGCCTGCGCCAACGCCTTCCAAAACAGCATTATCCCCGAGGACACAGACAAGACCCACACCTGTCCTCCATGTCCTGCTCCAGAACTGCTCGGCGGACCTTCCGTGTTCCTGTTTCCTCCAAAGCCTAAGGACACCCTGATGATCTCTCGGACCCCTGAAGTGACCTGCGTGGTGGTGGATGTGTCTCACGAGGATCCCGAAGTGAAGTTCAATTGGTACGTGGACGGCGTGGAAGTGCACAACGCCAAGACCAAGCCTAGAGAGGAACAGTACGGCTCCACCTACAGAGTGGTGTCCGTGCTGACAGTGCTGCACCAGGATTGGCTGAACGGCAAAGAGTACAAGTGCAAGGTGTCCAACAAGGCCCTGCCTGCCCCAATCGAAAAGACCATCTCCAAGGCCAAGGGCCAGCCTAGGGAACCCCAGGTTTACACCCTGCCTCCAAGCCGGGATGAGCTGACCAAGAACCAGGTGTCCCTGTCCTGCGCCGTCAAGGGCTTCTACCCTTCCGATATCGCCGTGGAATGGGAGAGCAATGGCCAGCCTGAGAACAACTACAAGACAACCCCTCCTGTGCTGGACTCCGACGGCTCATTCTTCCTGGTGTCCAAGCTGACCGTGGACAAGTCCAGATGGCAGCAGGGCAACGTGTTCTCCTGCTCCGTGATGCACGAGGCCCTGCACAATCACTACACCCAGAAGTCCCTGTCTCTGTCCCCTGGCAAA

SEQ ID NO: 60 - protein, 426 aa, synthetic construct
ANQVEQSPQSLIILEGKNVTLQCNYTVSPFSNLRWYKQDTGRGPVSLTILTFSENTKSNGRYTATLDADTKQSSLHITASQLSDSASYICVVNSAQGLYIPTFGRGTSLIVHPYIQKPDPAVYQLRDSKSSDKSVCLFTDFDSQTNVSQSKDSDVYITDKCVLDMRSMDFKSNSAVAWSNKSDFACANAFNNSIIPEDTDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 61 - protein, 504 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSSGGGGSDVKVTQSSRYLVKRTGEKVFLECVQDLDHENMFWYRQDPGLGLRLIYFSRFATGKEKGDIPEGYSVSREKKERFSLILESASTNQTSMYLCASSSDQNSGDPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 62 - protein, 253 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 63 - protein, 253 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSS

SEQ ID NO: 64 - protein, 199 aa, synthetic construct; identical to SEQ ID NO: 26

SEQ ID NO: 65 - protein, 130 aa, synthetic construct; identical to SEQ ID NO: 32

SEQ ID NO: 66 - protein, 107 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 67 - protein, 122 aa, synthetic construct
EVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSS

SEQ ID NO: 68 - protein, 6 aa, synthetic construct
QDIRNY

SEQ ID NO: 69 - skipped

SEQ ID NO: 70 - protein, 9 aa, synthetic construct
QQGNTLPWT

SEQ ID NO: 71 - protein, 8 aa, synthetic construct
GYSFTGYA

SEQ ID NO: 72 - protein, 8 aa, synthetic construct
INPYKGVS

SEQ ID NO: 73 - protein, 15 aa, synthetic construct
ARSGYYGDSDWYFDV

SEQ ID NO: 74 - protein, 122 aa, synthetic construct [sequence omitted]

SEQ ID NO: 75 - protein, 8 aa, synthetic construct
GYSFTGYT

SEQ ID NO: 76 - protein, 209 aa, synthetic construct
SVFLFPPKPKDTLYITREPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 77 - protein, 209 aa, synthetic construct
SVFLFPPKPKDTLYITREPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 10 aa, synthetic construct
ALWGPDPAAA

(19) United States
(12) Patent Application Publication
(10) Pub. No.: US 2026/0071001 A1
(43) Pub. Date: Mar. 12, 2026

(54) MULTI-DOMAIN BINDING MOLECULES

(71) Applicant: IMMUNOCORE LIMITED, Abingdon, Oxfordshire (GB)

(72) Inventors: Paul CONROY, Abingdon, Oxfordshire (GB); Stephen HEARTY, Abingdon, Oxfordshire (GB); Lok Hang MAK, Abingdon, Oxfordshire (GB); Tara MAHON, Abingdon, Oxfordshire (GB); David OVERTON, Abingdon, Oxfordshire (GB)

(21) Appl. No.: 19/104,630

(22) PCT Filed: Aug. 18, 2023
(86) PCT No.: PCT/EP2023/072835

§ 371 Date: Feb. 18, 2025

Related U.S. Application Data
(60) Provisional application No. 63/371,866, filed on Aug. 18, 2022.
(60) Provisional application No. 63/498,980, filed on Apr. 28, 2023.

(51) Int. Cl. C07K 16/30; A61K 39/00; A61P 35/00; C07K 14/725; C07K 16/28

(52) U.S. Cl. CPC C07K 16/30; A61P 35/00; C07K 14/7051; C07K 16/2833; A61K 2039/505; C07K 2317/41; C07K 2317/52; C07K 2317/53; C07K 2317/569; C07K 2317/75; C07K 2317/92; C07K 2317/94; C07K 2319/00

(57) ABSTRACT

The present invention relates to multi-domain binding molecules. The molecules comprise i) a peptide-major histocompatibility complex (pMHC) binding domain comprising a first variable region linked to a constant region (VC1) and a second variable region linked to a constant region (VC2), wherein VC1 and VC2 dimerise to form the pMHC binding domain; ii) an immune cell engaging domain comprising an antibody light chain variable domain (TCE-VL) linked to an antibody heavy chain variable domain (TCE-VH); and iii) a half-life extending domain comprising a first portion of an IgG Fc region (FC1) and a second portion of an IgG1 Fc region (FC2); wherein the multi-domain binding molecule comprises: a first polypeptide chain in which the immune cell engaging domain is linked to the N terminus of VC1; a second polypeptide chain in which VC2 is linked via its C terminus to the N terminus of FC1; and a third polypeptide chain comprising FC2; and wherein the pMHC binding domain and the immune cell engaging domain are capable of binding to a pMHC complex and a T cell respectively. The binding molecules can be used to treat diseases such as cancer, autoimmune diseases and infectious diseases.

The instant application contains a Sequence Listing and is hereby incorporated by reference in its entirety. Said XML copy, created on Aug. 8, 2023, is named P200739WO ST26 Sequence Listing.xml, and is 79,120 bytes in size.

The present invention relates generally to multi-domain binding molecules. The invention particularly relates to multi-domain binding molecules that comprise i) a peptide-major histocompatibility complex (pMHC) binding domain comprising a first variable region linked to a constant region (VC1) and a second variable region linked to a constant region (VC2), wherein VC1 and VC2 dimerise to form the pMHC binding domain; ii) an immune cell engaging (ICE) domain and iii) a half-life extending domain comprising a first portion of an IgG Fc region (FC1) and a second portion of an IgG1 Fc region (FC2); wherein the multi-domain binding molecule comprises: a first polypeptide chain in which the ICE domain is linked to the N terminus of VC1; a second polypeptide chain in which VC2 is linked via its C terminus to the N terminus of FC1; and a third polypeptide chain comprising FC2; and wherein the pMHC binding domain and the ICE domain are capable of binding to a pMHC complex and an immune cell respectively. The binding molecules can be used to treat diseases such as cancer, autoimmune diseases and infectious diseases.

### BACKGROUND TO THE INVENTION

Many protein-based therapeutics, including antibody fragments and fusion proteins, are rapidly cleared from the body following administration. Their short circulatory half-life is typically attributed to their small size, which allows for effective clearance via renal filtration, and lack of protection from intracellular degradation. In such cases, frequent administration or long infusion times are required to maintain an effective concentration of the drug over prolonged periods. To improve dosing, several strategies have been employed to extend circulatory half-life. These include increasing the hydrodynamic radius of the protein through attachment of flexible hydrophilic molecules, such as carbohydrate or PEG (polyethelene glycol), and exploiting recycling via the neonatal Fc receptor (FcRn), through attachment of antibody Fc domains or serum albumin (Konnteman, Curr Opin Biotechnol. 2011 December; 22(6):868-76).

Strategies that exploit FcRn mediated recycling are particularly attractive because of the lower risk of inducing immunogenicity in vivo and long half-life extensions that may be achieved. For example, the half-life of a T cell engaging bispecific antibody of the BiTE® format, is reported to be in excess of 200 h, following attachment of an Fc domain (Lorenczewski, et al., Blood 2017. 130(Suppl 1), 2815). Similarly, bispecific antibodies of the TriTac® format, which incorporate an albumin binding domain, are reported to have a half-life of over four days (Wesche et al., Cancer Res 2018; 78(13 Suppl):Abstract nr 3814).

Fusion proteins comprising a soluble T cell receptor (TCR) fused to an anti-CD3 antibody fragment, are a relatively new category of immune cell (e.g., T cell) engaging bispecific fusion proteins, with an in vivo half-life in the region of 6-8 h (Sato et al., 2018 J Clin Onc 2018 36, no. 15, suppl 9521-9521; Middleton et al., J Clin Onc 2016 34, no. 15, suppl 3016-3016). This is far shorter than traditional monoclonal antibodies, which typically have a half-life in the range of 260-720 hours (Ovacik & Lin, 2018 Clin Transl Sci, 11:540). There is therefore a need to identify suitable approaches for extending the half-life of TCR-immune cell engaging domain proteins such as TCR-anti-CD3 fusion proteins and other TCR-containing proteins. Furthermore TCR-anti-CD3 fusion proteins have demonstrated advantageous therapeutic properties including picomolar potency (Lowe et al. 2019 Cancer treatment reviews, vol. 77 35-43). There is therefore a need to identify suitable approaches for extending the half-life of TCR-anti-CD3 fusion proteins and other TCR-containing proteins in order to reduce dosing frequency and maintain effective concentrations over a prolonged period of time, without impacting other therapeutic properties.

Unlike traditional antibodies, TCRs are designed to recognise short peptides derived from intracellular antigens and presented on the cell surface by human leukocyte antigen (peptide-HLA). Effective immune synapse formation between a peptide-HLA complex on an antigen presenting cell and the corresponding receptor on an immune cell such as a T cell relies on carefully choreographed interactions which can be perturbed by increases in intermembrane distance, for example (Choudhuri et al., 2005 Nature July 28; 436(7050):578-82; Holland et al J Clin Invest. 2020; 130(5):2673-2688). Therefore, fusion approaches for increasing the half-life of TCR-containing proteins, such as attachment of antibody Fc domains or serum albumin, are highly challenging due to the risk of perturbing the interaction geometry required for TCR binding. Similar challenges also apply to fusion proteins containing antibodies that bind to peptide-HLA complexes, which are known as TCR-like or TCR-mimic antibodies.

WO 2020/157211 describes an approach for extending the half-life of a TCR-anti-CD3 fusion protein by fusing it to an immunoglobulin Fc domain or an albumin-binding domain. However, such multi-domain binding molecules are large and complex proteins, for which there are a myriad of possible formats, i.e., possible combinations of positions and orientations of each domain (and each region in each domain) on one or more polypeptide chains. The position and orientation of each domain (and regions thereof) in the molecule, and the number of polypeptide chains present, can influence characteristics of the binding molecule such as activity, half-life and manufacturability. Thus, there remains a need to identify favorable formats for such multi-domain binding molecules.

### DESCRIPTION OF THE INVENTION

As shown in Example 1, the inventors tested in excess of 40 different formats (i.e., orientations and positions of each domain in the polypeptide) for a multi-domain binding molecule comprising a pMHC binding domain, an immune cell engaging (ICE) domain (that can be an immune activator) and a half-life extending domain. In doing so, they found that, in many formats, fusing a TCR-anti-CD3 fusion protein to an Fc domain resulted in a substantial loss of potency in vitro. However, the inventors surprisingly identified a format (as depicted in FIGS. 1 and 10 herein) for such a molecule which retains a high degree of potency and specificity, and retains the therapeutic window of the original molecule. Examples 11-13 further show that this identified format is advantageous for a molecule comprising a different (pMHC) binding domain fused to a different ICE domain. The identified format works advantageously with TCRs binding to different targets, and with immune cell engaging domains that are both immune activators and immune suppressors.

In a first aspect, there is provided a multi-domain binding molecule comprising:
i) a peptide-major histocompatibility complex (pMHC) binding domain comprising a first variable region linked to a constant region (VC1) and a second variable region linked to a constant region (VC2), wherein VC1 and VC2 dimerise to form the pMHC binding domain;
ii) an immune cell engaging (ICE) domain; and
iii) a half-life extending domain comprising a first portion of an IgG Fc region (FC1) and a second portion of an IgG1 Fc region (FC2);
wherein the multi-domain binding molecule comprises:
a first polypeptide chain in which the ICE domain is linked to the N terminus of VC1;
a second polypeptide chain in which VC2 is linked via its C terminus to the N terminus of FC1; and
a third polypeptide chain comprising FC2; and
wherein the pMHC binding domain and the ICE domain are capable of binding to a pMHC complex and an immune cell respectively.

In one embodiment, there is provided a multi-domain binding molecule comprising:
i) a peptide-major histocompatibility complex (pMHC) binding domain comprising a first variable region linked to a constant region (VC1) and a second variable region linked to a constant region (VC2), wherein VC1 and VC2 dimerise to form the pMHC binding domain;
ii) a T cell engaging immune effector domain comprising an antibody light chain variable domain (TCE-VL) linked to an antibody heavy chain variable domain (TCE-VH); and
iii) a half-life extending domain comprising a first portion of an IgG Fc region (FC1) and a second portion of an IgG1 Fc region (FC2);
wherein the multi-domain binding molecule comprises:
a first polypeptide chain in which the T cell engaging immune effector domain is linked to the N terminus of VC1;
a second polypeptide chain in which VC2 is linked via its C terminus to the N terminus of FC1; and
a third polypeptide chain comprising FC2; and
wherein the pMHC binding domain and the T cell engaging immune effector domain are capable of binding to a pMHC complex and a T cell respectively.

Advantageously, the binding molecule of the invention has an extended in vitro half-life while retaining a similar therapeutic window and potent response to an equivalent non-Fc molecule. In yet a further aspect, there is provided one or more nucleic acids encoding one or more of the polypeptide chains of the multi-domain binding molecule according to the first aspect of the invention. There is also provided an expression vector comprising the nucleic acid or nucleic acids of this aspect. In addition, there is provided a host cell comprising the nucleic acid or the vector of this aspect.

Also provided, in a further aspect, is a method of making the multi-domain binding molecule according to the first aspect comprising maintaining the host cell described above under optimal conditions for expression of the nucleic acid and isolating the multi-domain binding molecule.

In a further aspect, there is provided a pharmaceutical composition comprising the multi-domain binding molecule according to the first aspect.

The multi-domain binding molecule, the nucleic acid, the vector, the host cell or the pharmaceutical composition of any of the above aspects may be used in the treatment of diseases such as cancer, autoimmune diseases and infectious diseases. Thus, in a further aspect, also provided is the multi-domain binding molecule, the nucleic acid, the vector, the host cell or the pharmaceutical composition for use as a medicament. In a still further aspect there is provided a method of treatment comprising administering the multi-domain binding molecule, the nucleic acid, the vector, the host cell or the pharmaceutical composition to a patient in need thereof.

Peptide-Major Histocompatibility Complex (pMHC) Binding Domains

A “pMHC binding domain”, as used herein, is a protein domain capable of binding to a peptide-MHC complex. A first variable region linked to a constant region (VC1) and a second variable region linked to a constant region (VC2) dimerise to form the pMHC binding domain. In this context, “VC1” refers to a region of the pMHC binding domain sequence that comprises the first variable region linked to a constant region and “VC2” refers to a region that comprises the second variable region linked to a constant region. The pMHC binding site is within the variable regions of VC1 and VC2. Suitable variable and constant region sequences include TCR or antibody variable and constant regions. The terms “MHC” and “HLA” as used herein are used interchangeably.

The pMHC binding domain may comprise at least part of a TCRα and a TCRβ chain. For example, the variable regions of VC1 and VC2 may be TCR variable regions. VC1 may comprise either a TCRα or a TCRβ variable region and VC2 may comprise the other of the TCRα and TCRβ variable regions. For example:
(i) VC1 may comprise either (a) a TCRα variable and constant region or (b) a TCRβ variable and constant region; and
(ii) VC2 may comprise the other of (a) or (b). Preferably, VC1 comprises the TCRβ variable and constant region and VC2 comprises the TCRα variable and constant region.

The pMHC binding domain may be a T cell receptor (TCR), such as a soluble TCR, comprising TCR variable regions, and constant regions. The TCR sequences defined herein are described with reference to IMGT nomenclature which is widely known and accessible to those working in the TCR field. For example, see: LeFranc and LeFranc, (2001). “T cell Receptor Factsbook”, Academic Press; Lefranc, (2011), Cold Spring Harb Protoc 2011 (6): 595-603; Lefranc, (2001), Curr Protoc Immunol Appendix 1: Appendix 100; and Lefranc, (2003), Leukemia 17(1): 260-266. Briefly, TCRs consist of two disulfide linked chains. Each chain (alpha and beta) is generally regarded as having two extracellular regions, namely a variable and a constant region. A short joining region connects the variable and constant regions and is typically considered part of the alpha variable region. Additionally, the beta chain usually contains a short diversity region next to the joining region, which is also typically considered part of the beta variable region. The variable region of each chain of a typical TCR is located N-terminally and comprises three Complementarity Determining Regions (CDRs) embedded in a framework sequence. The CDRs comprise the recognition site for peptide-MHC binding.

Alternatively, the pMHC binding domain may comprise variable regions of an antibody. The VC1 and VC2 variable regions may be antibody heavy or light chain variable regions. For example, VC1 may comprise either a heavy or a light chain antibody variable region and VC2 may comprise the other of the heavy or a light chain antibody variable region. In this regard, the pMHC binding domain may be a TCR-like antibody, also known as a “TCR mimic antibody” (TCRm-Ab). For example, the pMHC binding domain may comprise variable regions of a TCR-like antibody. Antibodies do not naturally recognise a pMHC complex. However, it is known that antibodies with specificity for pMHC can be engineered, as described in Chang et al., Expert Opin Biol Ther. 2016 August; 16(8):979-87 and Dahan et al., Expert Rev Mol Med. 2012 Feb. 24; 14:e6.

The pMHC binding domain may comprise constant regions. For example, VC1 and VC2 may each comprise a constant region. The constant region may correspond to a constant region from a TCRα chain or a TCRβ chain (TRAC or TRBC respectively). Alternatively the constant regions of the pMHC binding domain may be a constant region from an antibody light or heavy chain (for example it may comprise one or more or all of the CL, CH1, CH2, CH3 or CH4 domains). The constant region may be full length or may be truncated. TCR constant regions may be truncated to remove the transmembrane domain and cytoplasmic tail. Where the constant region is truncated, preferably only membrane-associated and cytoplasmic portions are removed from the C-terminal end. Where the pMHC binding domain comprises TCRα or TCRβ chain sequences, VC1 and VC2 may each comprise a TCR variable region and a TCR constant region. Preferably, VC1 and VC2 do not comprise a transmembrane or cytoplasmic domain, i.e., preferably the pMHC binding domain is soluble. Additional mutations may be introduced in to the amino acid sequence of the constant regions relative to natural constant regions. The constant regions may also include residues, either naturally-occurring or introduced, that allow for dimerisation by, for example, a disulphide bond between two cysteine residues.

If present, TCR portions of the molecules of the invention may be as heterodimers. Alpha-beta heterodimeric TCR portions of the molecules of the invention may comprise an alpha chain TRAC constant region sequence and/or a beta chain TRBC1 or TRBC2 constant region sequence. As described above, the constant regions may be in soluble format (i.e. having no transmembrane or cytoplasmic domains). One or both of the constant regions may contain mutations, substitutions or deletions relative to the native TRAC and/or TRBC1/2 sequences. The terms TRAC and TRBC1/2 also encompass natural polymorphic variants, for example N to K at position 4 of TRAC (Bragado et al International immunology. 1994 February; 6(2):223-30).

Alpha and beta chain constant region sequences may be modified by truncation or substitution to delete the native disulphide bond between Cys4 of exon 2 of TRAC and Cys2 of exon 2 of TRBC1 or TRBC2. Alpha and/or beta chain constant region sequence(s) may have an introduced disulphide bond between residues of the respective constant domains, as described, for example, in WO 2003/020763, WO 2004/033685 and WO 2006/000830. Alpha and beta constant regions may be modified by substitution of cysteine residues at position Thr 48 of TRAC and position Ser 57 of TRBC1 or TRBC2, the said cysteines forming a disulphide bond between the alpha and beta constant regions of the TCR. TRBC1 or TRBC2 may additionally include a cysteine to alanine mutation at position 75 of the constant domain and an asparagine to aspartic acid mutation at position 89 of the constant domain. One or both of the extracellular constant regions present in an as heterodimer may be truncated at the C terminus or C termini, for example by up to 15, or up to 10, or up to 8 or fewer amino acids. The C terminus of an alpha chain extracellular constant region may be truncated by 8 amino acids.

The amino acid sequence of the VC1 and VC2 variable and constant regions may correspond to those found in nature, or they may contain one or more mutations relative to a natural protein. Such mutations may be made to increase the affinity of the pMHC binding domain for a given antigen. Additionally or alternatively mutations may be incorporated to improve stability and manufacturability. The VC1 and VC2 sequences may be derived from human sequences.

The VC1 and VC2 sequences may comprise one or more engineered cysteine residues in the constant region to form a non-native disulfide bond between VC1 and VC2. Where VC1 and VC2 comprise TCR constant regions, suitable positions for introducing a disulfide bond between residues of the respective constant regions are described in WO 2003/020763 and WO 2004/033685. Single chain TCRs are further described in WO2004/033685; WO98/39482; WO01/62908; Weidanz et al. (1998) J Immunol Methods 221 (1-2): 59-76; Hoo et al. (1992) Proc Natl Acad Sci USA 89(10): 4759-4763; Schodin (1996) Mol Immunol 33(9): 819-829).

The VC1 may comprise a TCRα or TCRβ variable region and VC2 may comprise the other of the TCRα and TCRβ variable region. Preferably:
(i) the TCRα variable region comprises CDRs of SEQ ID NO: 2, 3, and 4 as CDR1, CDR2 and CDR3 respectively; and
(ii) the TCRβ variable region comprises CDRs of SEQ ID NO: 8, 9, and 10 as CDR1, CDR2 and CDR3 respectively.

Alternatively, the TCRα and TCRβ CDR sequences may each optionally have one, two, three, or four amino acid substitutions relative to the sequences recited above.

The TCRα variable region may comprise CDRs that are at least 90%, at least 95%, at least 98%, or at least 99% identical to the sequence of SEQ ID NO: 2, 3, and 4 as CDR1, CDR2 and CDR3 and/or the TCRβ variable region may comprise CDRs that are at least 90%, at least 95%, at least 98%, or at least 99% identical to SEQ ID NO: 8, 9, and 10 as CDR1, CDR2 and CDR3 respectively.

The TCRα variable region may be at least 80% identical to the sequence of SEQ ID NO: 5, 6 or 7 and the TCRβ variable region may be at least 80% identical to the sequence of SEQ ID NO: 11 or 12. The TCRα variable region may be at least 90%, at least 95%, or at least 98% identical to the sequence of SEQ ID NO: 5, 6 or 7 and the TCRβ variable region may be at least 90%, at least 95%, or at least 98% identical to the sequence of SEQ ID NO: 11 or 12. Preferably, the TCRα variable region has the sequence provided in SEQ ID NO: 5, 6 or 7 and the TCRβ variable region has the sequence provided in SEQ ID NO: 11 or 12. The TCRα variable region may have the sequence provided in SEQ ID NO: 5 and the TCRβ variable region may have the sequence provided in SEQ ID NO: 11. Alternatively, the TCRα variable region may have the sequence provided in SEQ ID NO: 6 and the TCRβ variable region may have the sequence provided in SEQ ID NO: 11. Alternatively, the TCRα variable region may have the sequence provided in SEQ ID NO: 7 and the TCRβ variable region may have the sequence provided in SEQ ID NO: 12.

VC1 may comprise a TCRα or TCRβ constant region and VC2 may comprise the other of the TCRα and TCRβ constant region. The TCRα constant region may be at least 80% identical to the sequence of SEQ ID NO: 29 and the TCRβ constant region may be at least 80% identical to the sequence of SEQ ID NO: 31. The TCRα constant region may be at least 90%, at least 95%, or at least 98% identical to the sequence of SEQ ID NO: 29 and the TCRβ constant region may be at least 90%, at least 95%, or at least 98% identical to the sequence of SEQ ID NO: 31. Preferably, the TCRα constant region has the sequence provided in SEQ ID NO: 29 and the TCRβ constant region has the sequence provided in SEQ ID NO: 31.

Alternatively, The TCRα constant region may be at least 80% identical to the sequence of SEQ ID NO: 30 and the TCRβ constant region may be at least 80% identical to the sequence of SEQ ID NO: 32. The TCRα constant region may be at least 90%, at least 95%, or at least 98% identical to the sequence of SEQ ID NO: 30 and the TCRβ constant region may be at least 90%, at least 95%, or at least 98% identical to the sequence of SEQ ID NO: 32. Preferably, the TCRα constant region has the sequence provided in SEQ ID NO: 30 and the TCRβ constant region has the sequence provided in SEQ ID NO: 32.

VC1 may comprise one of a TCRα variable and constant region or a TCRβ variable and constant region and VC2 may comprise the other of the TCRα and TCRβ variable and constant regions. The TCRα variable and constant region may comprise, or consist of, an amino acid sequence that is at least 80% identical to the sequence of SEQ ID NO: 24, 25 or 26 and the TCRβ variable and constant region may comprise, or consist of, an amino acid sequence that is at least 80% identical to the sequence of SEQ ID NO: 27 or 28. The TCRα variable and constant region may comprise, or consist of, an amino acid sequence that is at least 90%, at least 95%, or at least 98% identical to the sequence of SEQ ID NO: 24,25 or 26 and the TCRβ variable and constant region may comprise, or consist of, an amino acid sequence that is at least 90%, at least 95%, or at least 98% identical to the sequence of SEQ ID NO: 27 or 28. Preferably, the TCRα variable and constant region comprises, or consists of, the amino acid sequence provided in SEQ ID NO: 24, 25 or 26 and the TCRβ variable and constant region comprises, or consists of, the amino acid sequence provided in SEQ ID NO: 27 or 28.

The TCRα variable and constant region may have the sequence provided in SEQ ID NO: 24 and the TCRβ variable and constant region may have the sequence provided in SEQ ID NO: 27. Alternatively, the TCRα variable and constant region may have the sequence provided in SEQ ID NO: 25 and the TCRβ variable and constant region may have the sequence provided in SEQ ID NO: 27. Alternatively, the TCRα variable and constant region may have the sequence provided in SEQ ID NO: 26 and the TCRβ variable region and constant may have the sequence provided in SEQ ID NO: 28. The skilled person would appreciate that the format of the multi-domain binding molecule of the invention could equally be applied to TCR sequences other than those recited above. For example, other suitable TCR chain amino acid sequences are provided in WO2015092362, WO2011001152, WO2017109496, WO2017175006 and WO2018234319, and, for example, in U.S. Publ. No. 2016/0318988, and U.S. Pat. Nos. 8,519,100, 11,639,374, 11,505,590, and 11,427,624, the contents of each which are herein incorporated by reference.

A multi-domain binding molecule of the invention may have:
(a) a first polypeptide chain comprising in the following order, from N-terminus to C-terminus, an amino acid sequence of an immune cell engaging domain (e.g. an scFv or VHH domain), optionally followed by a linker sequence provided in SEQ ID NO: 34, and an amino acid sequence of a TCRβ variable and constant region;
(b) a second polypeptide chain comprising in the following order, from N-terminus to C-terminus, an amino acid sequence of a TCRα variable and constant region, optionally followed by a truncated hIgG1 hinge sequence provided in SEQ ID NO: 33 and an Fc region having the sequence provided in SEQ ID NO: 50;
(c) a third polypeptide chain comprising an Fc region having the sequence provided in SEQ ID NO: 52.

In a preferred embodiment, a multi-domain binding molecule of the invention may have:
(a) a first polypeptide chain comprising in the following order, from N-terminus to C-terminus, an amino acid sequence of an scFv, optionally followed by a linker sequence provided in SEQ ID NO: 34, and an amino acid sequence of a TCRβ variable and constant region;
(b) a second polypeptide chain comprising in the following order, from N-terminus to C-terminus, an amino acid sequence of a TCRα variable and constant region, optionally followed by a truncated hIgG1 hinge sequence provided in SEQ ID NO: 33 and an Fc region having the sequence provided in SEQ ID NO: 50;
(c) a third polypeptide chain comprising an Fc region having the sequence provided in SEQ ID NO: 52.

Preferably, the scFv is an anti-CD3 scFv.

In an alternative embodiment, a multi-domain binding molecule of the invention may have:
(a) a first polypeptide chain comprising in the following order, from N-terminus to C-terminus, an amino acid sequence of a VHH, optionally followed by a linker sequence provided in SEQ ID NO: 34, and an amino acid sequence of a TCRβ variable and constant region;
(b) a second polypeptide chain comprising in the following order, from N-terminus to C-terminus, an amino acid sequence of a TCRα variable and constant region, optionally followed by a truncated hIgG1 hinge sequence provided in SEQ ID NO: 33 and an Fc region having the sequence provided in SEQ ID NO: 50;
(c) a third polypeptide chain comprising an Fc region having the sequence provided in SEQ ID NO: 52.

Preferably, the VHH is a PD-1 agonist VHH.

In alternative embodiments, a multi-domain binding molecule of the invention may comprise a Fc domain having a first Fc region comprising the sequence of SEQ ID NO: 77 and a second Fc region comprising the sequence of SEQ ID NO: 76.

Thus, in any of the embodiments set out above, the Fc region having the sequence provided in SEQ ID NO: 50 may be replaced with an Fc region comprising the sequence provided in SEQ ID NO: 77 and the Fc region comprising the sequence provided in SEQ ID NO: 52 may be replaced with an Fc region having the sequence provided in SEQ ID NO: 76.

The TCRα chain and TCRβ chain may dimerise to form a peptide-major histocompatibility complex (pMHC) binding domain.

The anti-CD3 scFv may comprise, or consist of, the amino acid sequence provided in SEQ ID NO: 62 or the amino acid sequence provided in SEQ ID NO: 63.

The TCRβ constant region may have the amino acid sequence provided in SEQ ID NO: 31 and/or the TCRα constant region may have the amino acid sequence provided in SEQ ID NO: 29.

The multi-domain binding molecule may comprise no amino acid sequences other than the sequences in a) to c) above.

The TCRα chain and TCRβ chain may dimerise to form a peptide-major histocompatibility complex (pMHC) binding domain.

As is well-known in the art, protein molecules may be subject to post translational modifications. Glycosylation is one such modification, which comprises the covalent attachment of oligosaccharide moieties to defined amino acids in a TCR or antibody chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e. oligosaccharide type, covalent linkage and total number of attachments) can influence protein function. Therefore, when producing recombinant proteins, controlling glycosylation is often desirable. Controlled glycosylation has been used to improve antibody based therapeutics. (Jefferis et al., (2009) Nat Rev Drug Discov March; 8(3):226-34.). Glycosylation may be controlled, by using particular cell lines for example (including but not limited to mammalian cell lines such as Chinese hamster ovary (CHO) cells or human embryonic kidney (HEK) cells), or by chemical modification. Such modifications may be desirable, since glycosylation can improve pharmacokinetics, reduce immunogenicity and more closely mimic a native human protein (Sinclair and Elliott, (2005) Pharm Sci. August; 94(8):1626-35). Alternatively, glycosylation can lead to a lack of consistency in manufacturing which is not desirable for a therapeutic molecule. Residues at high risk of glycosylation, such as asparagine, may be substituted with an alternative amino acid, such as glutamine.

VC1 and/or VC2 may comprise one or more amino acid substitutions compared to unmodified V1 and/or VC2, wherein the one or more amino acid substitutions remove one or more glycosylation sites. The substitutions in this context are relative to a native (e.g., wild-type) sequence or unmodified sequence. For example:
(i) VC1 or VC2 may comprise a TCRα variable and constant region comprising one or more amino acid substitutions at positions selected from the group consisting of N18, N24, N146, N180 and N191, numbered according to SEQ ID NO: 25; and/or
(ii) the other of VC1 and VC2 may comprise a TCRβ variable and constant region comprising one or more amino acid substitutions at positions selected from the group consisting of N84 and 186, numbered according to SEQ ID NO: 27. The substitutions may be Asn to Gln (i.e., N to Q) substitutions. The TCRα variable and constant region may comprise N18Q, N24Q, N146Q, N180Q and N191Q substitutions, numbered according to SEQ ID NO: 25 and the TCRβ variable and constant region comprises a N84Q and N186Q substitution, numbered according to SEQ ID NO: 27.

The pMHC binding domain may not be fully aglycosylated, i.e., the pMHC may retain one or more glycosylation site(s) from its native sequence. For example, the pMHC binding domain may be glycosylated at a single glycosylation site (i.e., the pMHC binding domain may contain only one glycosylation site). The single glycosylation site may be in the variable region of VC1 or VC2. The single glycosylation site may be at position N18 of a TCRα variable region, numbered according to SEQ ID NO: 25.

The pMHC binding domain binds to MHC in complex with a peptide antigen. The peptide antigen may be a disease-associated antigen. The pMHC binding domain may bind to a tumour associated antigen peptide in complex with an MHC. For example, the peptide antigen may be a peptide derived from GP100, NYESO, MAGEA4, or PRAME as described in WO2011001152, WO2017109496, WO2017175006 and WO2018234319. The tumour associated antigen may be MAGEA4. Preferably, the pMHC binding domain binds to a GVYDGREHTV (SEQ ID NO: 1) HLA-A*02 complex.

In an alternative embodiment, the peptide antigen may be a peptide derived from a 3-cell antigen such as pre-pro-insulin (PPI). The ALWGPDPAAA₁₅₋₂₄ (SEQ ID No: 78) peptide is one such peptide derived from the signal sequence of human PPI (Skowera, et al. 2008 J Clin Invest. 118:3390-402 and WO2009004315). The peptide is loaded on to HLA-A*02 molecules and presented on the surface of insulin-producing p cells. Therefore, the ALWGPDPAAA-HLA-A*02 complex provides a human beta cell-specific marker that can be recognised by TCRs. High expression of this PPI peptide can be detected on the surface of beta cells, independent of disease stage, meaning a PPI targeted therapeutic could be efficacious at earlier disease stages compared to existing immunotherapies.

### Immune Cell Engaging Domains

An “immune cell engaging domain”, as used herein, is a protein domain that is capable of modifying an immune response, for example by promoting or suppressing an immune response such as T cell activation.

In some embodiments, the immune cell engaging domain comprises an antibody light chain variable region (TCE-VL) and an antibody heavy chain variable region (TCE-VH). As used herein, “TCE-VL” and “TCE-VH” refer to the light chain variable region and the heavy chain variable region of the immune cell engaging domain (ICE), respectively. “TCE-VL” and “TCE-VH” may also be referred to as “TCEVL” and “TCEVH” herein. Thus, the immune cell engaging domain may comprise an antigen-binding site. The immune cell engaging domain is also referred to herein as the “ICE” domain.

In some embodiments, the immune cell engaging domain may be a T cell engaging immune effector domain and may bind to a protein expressed on a cell surface of a T cell to promote activation of the T cell. For example, the T cell engaging immune effector domain may be a CD3 effector domain. The T cell engaging immune effector domain may bind to, for example, specifically bind to, CD3 (i.e., the T cell engaging immune effector domain may be a CD3-binding protein). The T cell engaging immune effector may be an antibody, or a functional fragment thereof, for example a single-chain variable fragment (scFv), or a similar sized antibody-like scaffold, or any other binding protein that activates a T cell through interaction with CD3 and/or the TCR/CD3 complex. The antibody may also be a single domain antibody, such as the variable region of a heavy chain antibody.

Alternatively, binding molecules of the invention may comprise an immune suppressor. As used herein, the term “immune suppressor” refers to any molecule, e.g., a protein, that is capable of inhibiting an immune response, such as inhibiting T cell activation. The immune suppressor may bind to a target (e.g. antigen). For example, the immune suppressor may be an immune checkpoint agonist, i.e., a molecule that induces immune checkpoint signalling. The immune suppressor may comprise an antigen-binding moiety that is capable of binding to an antigen. The antigen of the immune suppressor may be located on an immune cell, such as a T cell. The binding molecule may comprise an antibody or antigen binding fragment thereof, for example, the antibody may also be a single domain antibody, such as the variable region of a heavy chain antibody. Alternatively, the antibody may be a single-chain variable fragment (scFv), or a similar sized antibody-like scaffold, or any other binding protein that suppresses a T cell through induction of immune checkpoint signalling. Such immune suppressors are described below.

The immune cell engaging domain may be a single-chain variable fragment (scFv). “Single-chain Fv” also abbreviated as “sFv” or “scFv” are antibody fragments that comprise the VH and VL antibody domains connected into a single polypeptide chain. The scFv polypeptide may further comprise a polypeptide linker between the VH and VL domains which enables the scFv to form the desired structure for antigen binding. For a review of scFv's, see Pluckthun in The Pharmacology of Monoclonal Antibodies, vol. 113, Rosenburg and Moore eds., Springer-Verlag, New York, pp. 269-315 (1994).

The immune cell engaging domain may comprise an antigen-binding moiety that is capable of binding to an antigen. The antigen of the immune cell engaging domain may be located on an immune cell, such as a T cell. The binding molecule may comprise an antibody or antigen binding fragment thereof. The term “antibody” as used herein is meant to include conventional/native antibodies and engineered antibodies, in particular functional antibody fragments, single chain antibodies, single domain antibodies, and bispecific or multispecific antibodies. “Native” or “conventional”, in this context, refers to an antibody that has the same type of domains and domain arrangements as an antibody found in nature and comprises antibody-derived CDR and FR sequences. In a native/conventional four-chain, e.g. human, antibody, two heavy chains are linked to each other by disulfide bonds and each heavy chain is linked to a light chain by a disulfide bond. The variable domains of both light (VL) and heavy (VH) chains determine binding recognition and specificity to the antigen. Conventional antibody binding sites are made up of residues that are primarily from the “antibody complementarity determining regions” (CDRs) or hypervariable regions. Occasionally, residues from non-hypervariable or framework regions (FR) influence the overall domain structure and hence the binding site. CDRs refer to amino acid sequences that together define the binding affinity and specificity of the native antibody binding site. The light and heavy chains of a conventional four-chain antibody each have three CDRs, designated CDR1-L, CDR2-L, CDR3-L and CDR1-H, CDR2-H, CDR3-H, respectively. A conventional four-chain antibody antigen-binding site, therefore, includes six CDRs, comprising the CDR set from each of a VH and VL.

“Engineered” antibody formats include functional antibody fragments, single chain antibodies, single domain antibodies, and chimeric, humanized, bispecific or multispecific antibodies. Engineered antibody formats further include constructs in which TCR-derived CDRs, possibly including additional 3, 2 or 1 N and/or C terminal framework residues, or entire TCR-derived variable domains are grafted onto antibody heavy or light chains. A “functional antibody fragment” refers to a portion of a full-length antibody, or a protein that resembles a portion of a full-length antibody, that retains the ability to bind to its target antigen, in particular the antigen binding region or variable region of the full-length antibody. Examples of functional antibody “fragments” include Fv, Fab, F(ab′)2, Fab′, dsFv, (dsFv)2, scFv, sc(Fv)2 and diabodies. For example, a binding molecule of the invention may comprise a scFv.

The antibody may also be a single domain antibody, such as the variable region of a heavy chain antibody. In this regard, the term “single domain antibody” refers to an antibody that consists of a single antibody variable domain (e.g., a heavy chain variable domain). Thus, the immune suppressor may comprise a VHH (i.e., the variable domain of a heavy chain antibody), for example. As is known in the art, the antigen binding site of a single domain antibody, such as a VHH, may comprise three CDRs (as opposed to six in a conventional four-chain antibody). The term “antigen binding moiety of an antibody”, as used herein, encompasses such binding sites. Alternatively, or additionally, the binding molecule may comprise a Fab or Fv fragment. The term “Fab” (“fragment antigen-binding”) denotes an antigen-binding fragment of an antibody, which comprises the antibody light chain (VL-CL) and the variable and CH1 domain (VH-CH1) of the antibody heavy chain. Fab fragments typically have a molecular weight of about 50,000 Dalton. The Fv fragment is the N-terminal part of the Fab fragment of an antibody and consists of the variable portions of one light chain (VL) and one heavy chain (VH).

The immune cell engaging domain may comprise an antibody heavy chain variable region (VH) and an antibody light chain variable region (VL), which associate to form the antigen-binding moiety that is capable of binding to the antigen. Thus, the antigen binding moiety may comprise the VH and the VL. For example, the immune cell engaging domain may comprise a scFv comprising the VH and VL. Other suitable antigen binding moieties are heavy chain antibodies (hcAb), single domain antibodies (sdAb), minibodies (Tramontano et al (1994) J. Mol. Recognition 7, 9-24), the variable domain of camelid heavy chain antibodies (VHH), the variable domain of the new antigen receptors (VNAR), affibodies (Nygren P. A. (2008) FEBS J. 275, 2668-2676), alphabodies (see WO2010066740), designed ankyrin-repeat domains (DARPins) (Stumpp et al (2008) Drug Discovery Today 13, 695-701), anticalins (Skerra et al (2008) FEBS J. 275, 2677-2683), knottins (Kolmar et al (2008) FEBS J. 275, 2684-2690) and engineered CH2 domains (nanoantibodies, see Dimitrov D S (2009) mAbs 1, 26-28).

The antigen binding moiety may be, or comprise, a heavy chain variable domain that comprises, consists or essentially consists of four framework regions (FR1 to FR4 respectively) and three complementarity determining regions (CDR1 to CDR3 respectively); or any suitable fragment of such a heavy chain variable domain (which retains the antigen binding site). The antigen binding moiety may be a heavy chain antibody. The antigen binding moiety may be a heavy chain variable domain sequence of an antibody that is derived from a conventional four-chain antibody, such as, without limitation, a VH sequence that is derived from a human antibody. Preferably, the antigen binding moiety is, or comprises, the variable domain of a heavy chain antibody (e.g., a camelid antibody), such as a VHH (also referred to herein as a “VHH domain”). Preferably, the antigen binding moiety is a VHH.

As described herein, the immune cell engaging domain may comprise an antigen binding moiety (e.g., an antibody antigen binding moiety) that binds to an antigen located on an immune cell. In the context of the present invention, “immune cell” may refer to, for example, a T cell or a B cell. In particular, the antigen of the antigen-binding moiety may be a T cell surface antigen.

CD3 effectors include but are not limited to anti-CD3 antibodies or antibody fragments, in particular an anti-CD3 scFv or antibody-like scaffolds. The immune cell engaging domain may be a T cell engaging immune effector domain, which may be an anti-CD3 scFv. Further immune effectors include but are not limited to antibodies, including fragments, derivatives and variants thereof, that bind to antigens on immune cells such as T cells. Such antigens include CD28, 4-1bb (CD137) or CD16 or any molecules that exert an effect at the immune synapse. A particularly preferred immune effector is an anti-CD3 antibody, or a functional fragment or variant of said anti-CD3 antibody. As used herein, the term “antibody” encompasses such fragments and variants. Examples of anti-CD3 antibodies include but are not limited to OKT3, UCHT-1, BMA-031 and 12F6. Antibody fragments and variants/analogues which are suitable for use in the compositions and methods described herein include minibodies, Fab fragments, F(ab′)2 fragments, dsFv and scFv fragments.

Preferably, the immune cell engaging domain comprises:
(i) a VL region comprising CDRs of SEQ ID NO: 68, 69, and 70 as CDR1, CDR2 and CDR3 respectively; and
(ii) a VH region comprising CDRs of SEQ ID NO: 71, 72, and 73 as CDR1, CDR2 and CDR3 respectively.

Alternatively, the immune cell engaging domain may comprise:
(i) a VL region comprising CDRs of SEQ ID NO: 68, 69, and 70 as CDR1, CDR2 and CDR3 respectively; and
(ii) a VH region comprising CDRs of SEQ ID NO: 75, 72, and 73 as CDR1, CDR2 and CDR3 respectively.

The VL and VH CDR sequences above may each optionally have one, two, three, or four amino acid substitutions relative to the sequences recited above.

The TCE-VL may comprise CDRs that are at least 90%, at least 95%, at least 98%, or at least 99% identical to the sequence of SEQ ID NO: 68, 69, and 70 as CDR1, CDR2 and CDR3 and/or the TCE-VH may comprise CDRs that are at least 90%, at least 95%, at least 98%, or at least 99% identical to SEQ ID NO: 71, 72, and 73 as CDR1, CDR2 and CDR3 respectively.

Alternatively, the TCE-VL may comprise CDRs that are at least 90%, at least 95%, at least 98%, or at least 99% identical to the sequence of SEQ ID NO: 68, 69, and 70 as CDR1, CDR2 and CDR3 and/or the TCE-VH may comprise CDRs that are at least 90%, at least 95%, at least 98%, or at least 99% identical to SEQ ID NO: 75, 72, and 73 as CDR1, CDR2 and CDR3 respectively.

The TCE-VL may comprise, or consist of, an amino acid sequence that is at least 80% identical to the sequence of SEQ ID NO: 66 and the TCE-VH may comprise, or consist of, an amino acid sequence that is at least 80% identical to the sequence of SEQ ID NO: 67. The TCE-VL may comprise, or consist of, an amino acid sequence that is at least 90%, at least 95%, or at least 98% identical to the sequence of SEQ ID NO: 66 and the TCE-VH may comprise, or consist of, an amino acid sequence that is at least 90%, at least 95%, or at least 98% identical to the sequence of SEQ ID NO: 67. Preferably, the TCE-VL comprises, or consists of, the amino acid sequence provided in SEQ ID NO: 66 and the TCE-VH comprises, or consists of, the amino acid sequence provided in SEQ ID NO: 67.

Alternatively, the TCE-VL comprises, or consists of, an amino acid sequence that is at least 80% identical to the sequence of SEQ ID NO: 66 and the TCE-VH comprises, or consists of, an amino acid sequence that is at least 80% identical to the sequence of SEQ ID NO: 74. The TCE-VL may comprise, or consist of, an amino acid sequence that is at least 90%, at least 95%, or at least 98% identical to the sequence of SEQ ID NO: 66 and the TCE-VH may comprise, or consist of, an amino acid sequence that is at least 90%, at least 95%, or at least 98% identical to the sequence of SEQ ID NO: 74. For example, the TCE-VL may comprise, or consist of, the amino acid sequence provided in SEQ ID NO: 66 and the TCE-VH may comprise, or consist of, the amino acid sequence provided in SEQ ID NO: 74.

As described above, the immune cell engaging domain may be an scFv. The immune cell engaging domain may be an scFv comprising, or consisting of, an amino acid sequence that is at least 80% identical to the sequence of SEQ ID NO: 62 or 63. The scFv may comprise, or consist of, an amino acid sequence that is at least 90%, at least 95%, or at least 98% identical to the sequence of SEQ ID NO: 62 or 63. Preferably, the scFv comprises, or consists of, the amino acid sequence provided in SEQ ID NO: 63. Alternatively, the scFv may comprise, or consist of, the amino acid sequence provided in SEQ ID NO: 62.

The target (i.e., antigen) of the immune cell engaging domain may alternatively be an immune suppressor. For example, the target may be an immune checkpoint molecule, such as PD-1 (Programmed Death 1 receptor), A2AR (Adenosine A2A receptor), A2BR (Adenosine A2B receptor), B7-H3 (B7 Homolog 3, also called CD276) B7-H4 (B7 Homolog 4, also called VTCN1), BTLA (B and T Lymphocyte Attenuator, also called CD272), CTLA-4 (Cytotoxic T-Lymphocyte-Associated protein 4, also called CD152), IDO (Indoleamine 2,3-dioxygenase), CD200 Receptor, KIR (Killer-cell Immunoglobulin-like Receptor), TIGIT (T cell Immunoreceptor with Ig and ITIM domains), LAG3 (Lymphocyte Activation Gene-3), NOX2 (nicotinamide adenine dinucleotide phosphate NADPH oxidase isoform 2), TIM-3 (T-cell Immunoglobulin domain and Mucin domain 3), VISTA (V-domain Ig suppressor of T cell activation), SIGLEC7 (Sialic acid-binding immunoglobulin-type lectin 7, also called CD328), and SIGLEC9 (Sialic acid-binding immunoglobulin-type lectin 9, also called CD329).

In this regard, the immune suppressor may be an agonist of one or more of the above immune checkpoint molecules. Thus, the immune suppressor may be an immune checkpoint agonist (i.e., to inhibit immune activation). Suitable immune checkpoint agonists, including native ligands and antibodies, are reviewed in Paluch et al Front Immunol, 2018, 9:2306, for example.

Instead of comprising an antigen binding moiety of an antibody, the immune suppressor may comprise one of a receptor-ligand pair, whereby the immune suppressor is capable of binding to the other of the receptor-ligand pair. The target ligand or receptor may be located on an immune cell. For example, the immune suppressor may comprise a ligand of an immune checkpoint molecule described above. In particular, the immune suppressor may comprise a portion (e.g., a soluble extracellular region) of PD-L1 that is capable of binding to PD-1. Such an immune suppressor may engage an immune cell by binding to PD-1 and stimulate PD-1 signalling.

Alternatively the immune suppressor may comprise an agonist antibody that binds to, and preferably stimulates signalling of, an immune checkpoint molecule. For example, the immune suppressor may be, or comprise, a PD-1 agonist antibody (e.g., single domain antibody). Preferably, such PD-1 agonists do not compete with PD-L1 for binding to PD-1. The PD-1 agonist may a full-length antibody or fragment thereof, such as a scFv antibody or a Fab fragment, or a single domain antibody. Examples of such antibodies are provided in WO2011110621 and WO2010029434 and WO2018024237. Thus the antigen of the immune suppressor may be PD-1 and the antigen binding moiety of the immune suppressor may be a PD-1 agonist. The antigen binding moiety of the immune suppressor may comprise a single domain antibody, optionally a VHH. Preferably, the immune suppressor is a PD-1 agonist VHH.

The immune suppressor is preferably a PD-1 agonist. As used herein, the term “PD-1 agonist” refers to any molecule that is capable of binding to PD-1 and activating PD-1 signalling, including e.g., the PD-1 ligand, PD-L1, and PD-1 agonist antibodies. Activation of the PD-1 pathway down-regulates immune activity, promoting peripheral immune tolerance and preventing autoimmunity (Keir et al., Annu Rev Immunol, 26:677-704, 2008; Okazaki et al., Int Immunol 19:813-824, 2007).

### Half-Life Extending Domains

A “half-life extending domain”, as used herein, refers to a protein domain for extending the half-life of the multi-domain binding protein, relative to a multi-domain binding protein lacking the half-life extending domain. The half-life extending domain comprises a first IgG Fc region (FC1) and a second IgG Fc region (FC2), wherein the FC1 region and FC2 region dimerise to form an Fc domain. As used herein, the term “Fc region” is used to refer to a region of a single polypeptide chain comprising at least a CH2 domain and a CH3 domain sequence, whereas the term “Fc domain” refers to a dimer of two Fc regions (i.e., FC1 and FC2).

The term “half-life” as used herein means a pharmacokinetic property of a binding molecule that is a measure of the mean survival time of binding molecules following their administration. Binding molecule half-life can be expressed as the time required to eliminate 50 percent of a known quantity of a binding molecule from the patient's body (or other mammal) or a specific compartment thereof, for example, as measured in serum, i.e., circulating half-life, or in other tissues.

An increase in half-life allows for the reduction in amount of drug given to a patient as well as reducing the frequency of administration. An increase in half-life can be beneficial, for example, for treatment of cancer, infectious disease or an autoimmune disease or condition. Binding proteins with increased half-lives may also be generated by modifying amino acid residues identified as involved in the interaction between the Fc and the FcRn receptor. Binding proteins comprising Fc regions that comprise one or more modifications which promote binding to FcRn may have an increased half-life of about 5%, about 10%, about 15%, about 20%, about 25%, about 30%, about 35%, about 40%, about 45%, about 50%, about 60%, about 65%, about 70%, about 80%, about 85%, about 90%, about 95%, about 100%, about 125%, about 150% or more as compared to a binding protein comprising a native Fc region. Binding proteins comprising Fc regions that comprise one or more modifications which promote binding to FcRn may have an increased half-life of about 2 fold, about 3 fold, about 4 fold, about 5 fold, about 10 fold, about 20 fold, about 50 fold or more, or is between 2 fold and 10 fold, or between 5 fold and 25 fold, or between 15 fold and 50 fold, as compared to binding proteins comprising a native Fc region.

WO 2020/157211 describes an approach for extending the half-life of a TCR-anti-CD3 fusion protein by fusing it to an IgG Fc domain. The present inventors have surprisingly found that the multi-domain binding molecules of the invention retain the extended half-life provided by the Fc domain in the format disclosed in WO 2020/157211, but, in addition, have significantly higher potency.

The immunoglobulin Fc domain may be any antibody Fc domain. The Fc domain is the tail region of an antibody that interacts with cell surface Fc receptors and some proteins of the complement system. The Fc domain comprises two polypeptide chains (i.e., two Fc “regions”) both having two or three heavy chain constant domains (termed CH2, CH3 and CH4), and optionally a hinge region. The two Fc region chains may be linked by one or more disulphide bonds within the hinge region. Fc domains from immunoglobulin subclasses IgG1, IgG2 and IgG4 bind to and undergo FcRn mediated recycling, affording a long circulatory half-life (3-4 weeks), thus extending the half-life of the multi-domain binding molecule of the invention. The interaction of IgG with FcRn has been localized in the Fc region covering parts of the CH2 and CH3 domains. Preferred immunoglobulin Fc domains for use in the present invention include, but are not limited to Fc domains from IgG1 or IgG4. For example, the Fc domain may be an IgG1 Fc domain, i.e., the FC1 and FC2 regions may be IgG1 Fc regions. The Fc domain may be derived from human sequences.

The FC1 region may comprise, or consist of, an amino acid sequence that is at least 80% identical to the sequence of SEQ ID NO: 50 and the FC2 region may comprise, or consist of, an amino acid sequence that is at least 80% identical to the sequence of SEQ ID NO: 49. The FC1 region may comprise, or consist of, an amino acid sequence that is at least 90%, at least 95%, or at least 98% identical to the sequence of SEQ ID NO: 50 and the FC2 region may comprise, or consist of, an amino acid sequence that is at least 90%, at least 95%, or at least 98% identical to the sequence of SEQ ID NO: 49. Preferably, the FC1 region comprises, or consists of, the amino acid sequence provided in SEQ ID NO: 50 and the FC2 region comprises, or consists of, the amino acid sequence provided in SEQ ID NO: 49. As the skilled person would appreciate, the sequences provided above for FC1 and FC2 are suitable vice versa. For example, the FC1 region may comprise, or consist of, the amino acid sequence provided in SEQ ID NO: 49 and the FC2 region may comprise, or consist of, the amino acid sequence provided in SEQ ID NO: 50.

The Fc regions may comprise mutations relative to a wild-type or unmodified Fc sequence. Mutations include substitutions, insertions and deletions. Such mutations may be made for the purpose of introducing desirable therapeutic properties. For example, to facilitate hetero-dimerisation, knobs into holes (KiH) mutations maybe engineered into the CH3 domain. Thus, the half-life extending domain may comprise one or more amino acid substitutions which facilitate dimerisation of the FC1 region and the FC2 region. Such substitutions include “Knob-in-hole” substitutions. In this case, one chain (i.e. one of the FC1 or FC2 regions) is engineered to contain a bulky protruding residue (i.e. the knob), such as Y, and the other chain (i.e., the other of the FC1 and FC2 regions) is engineered to contain a complementary pocket (i.e. the hole). For example, a knob may be constructed by replacing a small amino acid side chain with a larger side chain. A hole may be constructed by replacing a large amino acid side chain with a smaller side chain. Without wishing to be bound to theory, this is thought to stabilize a hetero-dimer of the FC1 and FC2 regions by favouring formation of the hetero-dimer over other species, for example homomultimers of FC1 and FC2, thereby enhancing the stability and manufacturability of the multi-domain binding molecule of the invention.

Suitable positions and substitutions for KiH mutations, and other mutations for facilitating dimerisation of Fc regions, are known in the art and include those described in Merchant et al., Nat Biotechnol 16:677 (1998) and Ridgway et al., Prot Engineering 9:617 (1996) and Atwell et al. J Mol Biol 270, 1 (1997): 26-35. For example, the substitutions forming corresponding knobs and holes in two Fc regions may correspond to one or more pairs provided in the following table:

| CH3 of one of the FC1 and | CH3 of the other of the |
|---|---|
| FC2 regions | FC1 and FC2 regions |
| T366Y | Y407T |
| T366W | Y407A |
| T366W | T366S:L368A:Y407V |
| F405A | T394W |
| Y407T | T366Y |
| T366Y:F405A | T394W:Y407T |
| T366W:F405W | T394S:Y407A |
| F405W:Y407A | T366W:T394S |
| F405W | T394S |

The substitutions in the table above are denoted by the original residue, followed by the position using the EU numbering system, and then the import residue (all residues are given in single-letter amino acid code). Multiple substitutions are separated by a colon.

The FC1 and FC2 regions may comprise one or more substitutions in the table above. For example:
(i) one of the FC1 region and the FC2 region may comprise one or more amino acid substitutions selected from the group consisting of T366S, L368A, T394S, F405A, Y407A, Y407T and Y407V, according to the EU numbering scheme; and
(ii) the other of the FC1 region and the FC2 region may comprise one or more amino acid substitutions selected from the group consisting of T366W, T366Y, T366W, T394W and F405W according to the EU numbering scheme. The substitutions in (i) and (ii) are hole-forming and knob-forming substitutions respectively. The FC1 region may comprise one or more of the substitutions in (i) and the FC2 region may comprise one or more of the substitutions in (ii).

For example:
(i) one of the FC1 region and the FC2 region may comprise one or more amino acid substitutions selected from the group consisting of T366S, L368A, and Y407V, according to the EU numbering scheme; and
(ii) the other of the FC1 region and the FC2 region may comprise a T366W amino acid substitution, according to the EU numbering scheme. The FC1 region may comprise one or more of the substitutions in (i) and the FC2 region may comprise the substitution in (ii).

Preferably, (i) one of the FC1 region and the FC2 region comprises T366S, L368A, and Y407V amino acid substitutions, according to the EU numbering scheme; and (ii) the other of the FC1 region and the FC2 region comprises a T366W amino acid substitution, according to the EU numbering scheme. For example, the FC1 region may comprise a T366W amino acid substitution, according to the EU numbering scheme; and the FC2 region may comprise T366S, L368A, and Y407V amino acid substitutions, according to the EU numbering scheme.

The Fc domain may also comprise one or more mutations that attenuate an effector function of the Fc domain. Exemplary effector functions include, without limitation, complement-dependent cytotoxicity (CDC) and/or antibody-dependent cellular cytotoxicity (ADCC). The modification to attenuate effector function may be a modification that alters the glycosylation pattern of the Fc domain, e.g., a modification that results in an aglycosylated Fc domain. Alternatively, the modification to attenuate effector function may be a modification that does not alter the glycosylation pattern of the Fc domain. The modification to attenuate effector function may reduce or eliminate binding to human effector cells, binding to one or more Fc receptors, and/or binding to cells expressing an Fc receptor. For example, the half-life extending domain may comprise one or more amino acid substitutions selected from the group consisting of S228P, E233P, L234A, L235A, L235E, L235P, G236R, G237A, P238S, F241A, V264A D265A, H268A, D270A, N297A, N297G, N297Q, E318A, K322A, L328R, P329G, P329A, A330S, A330L, P331A and P331S, according to the EU numbering scheme. Particular modifications include a N297G or N297A substitution in the Fc region of human IgG1 (EU numbering). Other suitable modifications include L234A, L235A and P329G substitutions in the Fc region of human IgG1 (EU numbering), that result in attenuated effector function. The Fc regions in the multi-domain binding molecule of the invention may comprise a substitution at residue N297, numbering according to EU index. For example, the substitution may be an N297G or N297A substitution. Other suitable mutations (e.g., at residue N297) are known to those skilled in the art.

Fc variants having reduced effector function refers to Fc variants that reduce effector function (e.g., CDC, ADCC, and/or binding to FcR, etc. activities) by at least 10%, 20%, 30%, 40%, 50%, 60%, 70%, 80%, 90%, 95%, 97%, 98%, 99% or more as compared to the effector function achieved by a wild-type Fc region (e.g., an Fc region not having a mutation to reduce effector function, although it may have other mutations). The Fc variants having reduced effector function may be Fc variants that eliminate all detectable effector function as compared to a wild-type Fc region. Assays for measuring effector function are known in the art and described below.

In vitro and/or in vivo cytotoxicity assays can be conducted to confirm the reduction/depletion of CDC and/or ADCC activities. For example, Fc receptor (FcR) binding assays can be conducted to ensure that the Fc region or fusion protein lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)).

Substitutions may be introduced into the FC1 and FC2 regions that abrogate or reduce binding to Fcy receptors and/or increase binding to FcRn, and/or prevent Fab arm exchange, and/or remove protease sites. In this regard, the half-life extending domain may also comprise one or more amino acid substitutions which prevent or reduce binding to activating receptors. The half-life extending domain may comprise one or more amino acid substitutions which prevent or reduce binding to FcγR. For example, the FC1 region and/or the FC2 region may comprise a N297G amino acid substitution, according to the EU numbering scheme. Both the FC1 region and the FC2 region may comprise the N297G amino acid substitution.

The half-life extending domain may comprise one or more amino acid substitutions compared to the unmodified half-life extending domain, wherein the one or more amino acid substitutions promote binding to FcRn. Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody substitutions which improved or diminished binding to FcRs. See also, e.g., Shields et al., J. Biol. Chem. 9(2): 6591-6604 (2001). In particular, Mackness et al., MAbs. 11:1276-1288 (2019) describes suitable amino acid substitutions in antibody Fc regions for enhancing binding to FcRn.

The modification(s) (e.g., amino acid substitutions, amino acid insertions, or amino acid deletions) in the Fc region which promote binding to FcRn may be at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 331, 332, 333, 334, 339, 341, 343, 370, 373, 378, 392, 416, 419, 421, 440 and 443 as numbered by the EU index as set forth in Kabat. Optionally, the Fc region may comprise a non-naturally occurring amino acid residue at additional and/or alternative positions known in the art.

More specifically, the Fc region can comprise at least one substitution selected from the group consisting of 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 240I, 240A, 240T, 240M, 241W, 241 L, 241Y, 241E, 241R. 243W, 243L 243Y, 243R, 243Q, 244H, 245A, 247L, 247V, 247G, 251F, 252Y, 254T, 255L, 256E, 256M, 262I, 262A, 262T, 262E, 263I, 263A, 263T, 263M, 264L, 264I, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 305I, 313F, 316D, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 331G, 331A, 331L, 331M, 331F, 331W, 331K, 331Q, 331E, 331S, 331V, 331I, 331C, 331Y, 331H, 331R, 331N, 331D, 331T, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, the Fc region may comprise additional and/or alternative non-naturally occurring amino acid residues known in the art.

The modification(s) (e.g., amino acid substitutions, amino acid insertions, or amino acid deletions) in the Fc region which promote binding to FcRn may be at one or more positions selected from the group consisting of 234, 235 and 331, as numbered by the EU index as set forth in Kabat. For example, the Fc region may comprise at least one substitution selected from the group consisting of 234F, 235F, 235Y, and 331S, as numbered by the EU index as set forth in Kabat.

The modification(s) (e.g., amino acid substitutions, amino acid insertions, or amino acid deletions) in the Fc region which promote binding to FcRn may be at one or more positions selected from the group consisting of 239, 330 and 332, as numbered by the EU index as set forth in Kabat. For example, the Fc region may comprise at least one substitution selected from the group consisting of 239D, 330L and 332E, as numbered by the EU index as set forth in Kabat.

The modification(s) (e.g., amino acid substitutions, amino acid insertions, or amino acid deletions) in the Fc region which promote binding to FcRn may be at one or more positions selected from the group consisting of 252, 254, and 256, as numbered by the EU index as set forth in Kabat. For example, the Fc region may comprise at least one substitution selected from the group consisting of 252Y, 254T and 256E, as numbered by the EU index as set forth in Kabat, as described in U.S. Pat. No. 7,083,784, the contents of which are herein incorporated by reference in its entirety. The Fc region may comprise all of the following substitutions: 252Y, 254T and 256E, as numbered by the EU index as set forth in Kabat.

The substitutions which promote binding to FcRn listed above are relative to a corresponding wild-type Fc region (e.g., a human IgG1 or IgG4 Fc region) and may be present in one of or preferably both of the FC1 and FC2 portions of the Fc region. In other words, the substitutions refer to amino acids that are not normally present in a corresponding wild-type Fc region, for example a human IgG1 or IgG4 Fc region. In this regard, a “substitution”, as used herein, refers to the presence of one of the listed amino acids in a polypeptide and does not necessarily require replacing one amino acid with another

In one embodiment, the FC1 and/or FC2 region comprise the M252Y/S254T/T256E amino acid substitutions (numbered according to the EU numbering scheme).

Additionally or alternatively, mutations may be made for manufacturing reasons, for example to remove or replace amino acids that may be subject to post-translational modifications such as glycosylation, as described herein. The immunoglobulin Fc may be fused to the other domains (i.e., VC1 or VC2) in the molecule of the invention via a linker, and/or a hinge sequence as described herein. Alternatively no linker may be used.

The two Fc regions in the molecule of the invention may comprise CH2 and CH3 constant domains and all or part of a hinge sequence. The hinge sequence may correspond substantially or partially to a hinge region from IgG1, IgG2, IgG3 or IgG4. The hinge sequence may be an IgG1 hinge sequence, such as the amino acid sequence provided in SEQ ID NO: 33. The hinge may comprise all or part of a core hinge domain and all or part of a lower hinge region.

### Format and Linkers

As used herein, the term “format” refers to the position and orientation of each domain (and each region in each domain), and the number of polypeptide chains, in the multi-domain binding molecule of the invention. A schematic diagram of the format of an exemplary multi-domain binding molecule is provided in FIG. 1. The pMHC binding domain and the immune cell engaging (ICE) domain of such molecules are capable of binding to a pMHC complex and a T cell, respectively. In this regard, the pMHC binding domain and the immune cell engaging (ICE) domain may be capable of simultaneously binding to a pMHC complex and a T cell, respectively.

The multi-domain binding molecule format of the invention comprises three polypeptide chains: a first polypeptide chain in which an T cell engaging immune effector domain is linked to the N terminus of VC1; a second polypeptide chain in which a VC2 is linked via its C terminus to the N terminus of the FC1; and a third polypeptide chain comprising FC2.

The format can be represented as: N-(TCEVL-TCEVH or TCEVH-TCEVL)-VC1; N-VC2-FC1-C; N-FC2-C. The inventors have identified that molecules in this format have the highest activity (i.e., potency and selectivity) of the in excess of 40 formats different formats tested.

The multi-domain binding molecule of the invention is in a three-chain format. In this context, three-chain” is used to describe a multi-domain binding molecule that is expressed as three separate polypeptide chains which associate with each other to form a single three-dimensional folded structure comprising a pMHC binding domain, the immune cell engaging (ICE) domain and the half-life extending domain.

Preferably, VC1 comprises a TCRβ variable and constant region, VC2 comprises a TCRα variable and constant region, the immune cell engaging domain (ICE) is an anti-CD3 scFv and the Fc domain is an IgG1 Fc domain.

The TCE-VH, TCE-VL, VC1, VC2, FC1 and FC2 regions may be linked to each other via linkers and/or IgG hinge sequences. Linker sequences may be flexible, in that they are made up primarily of amino acids such as glycine, alanine and serine, which do not have bulky side chains likely to restrict flexibility. Such linkers include “glycine-serine” linkers, which refer to linkers that comprise only, or predominantly, glycine and serine residues for example (GGGGS)n). Alternatively, linkers with greater rigidity may be desirable. Examples of more rigid linkers include alpha helix-forming linkers with the sequence of (EAAAK)n. Usable or optimum lengths of linker sequences may be easily determined. Often the linker sequence will be less than about 15, such as less than 10, or from 2-10 amino acids in length. The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used in multi-domain binding molecules are known in the art and include those described in WO2010/133828 and Chen et al Adv Drug Deliv Rev. 2013; 65(10):1357-1369. For example, the linker or linkers present in the multi-domain binding protein of the invention may have a sequence selected from the group of GGGGS (SEQ ID NO: 34), GGGSG (SEQ ID NO: 35), GGSGG (SEQ ID NO: 36), GSGGG (SEQ ID NO: 37), GSGGGP (SEQ ID NO: 38), GGEPS (SEQ ID NO: 39), GGEGGGP (SEQ ID NO: 40), GGEGGGSEGGGS (SEQ ID NO: 41), and GGGSGGGG (SEQ ID NO: 42). Further suitable linkers include GGGGSGGGGSGGGGSGGGGS (SEQ ID NO: 43), EAAAK (SEQ ID NO: 44) and EAAAKEAAAKEAAAK (SEQ ID NO: 45), GGGGSGGGGSGGGGSGGGGSGGGS (SEQ ID NO: 46)

Suitable IgG hinge sequences are known in the art and include the exemplary truncated IgG1 hinge sequence provided in SEQ ID NO: 33. Other suitable IgG hinge sequences include a full IgG1 hinge sequence provided in SEQ ID NO: 47 and the IgG4 hinge provided in SEQ ID NO: 48.

The TCE-VL region may be linked via its C terminus to the N terminus of the TCE-VH region and the TCE-VH region may be linked via its C terminus to the N terminus of VC1. In this regard, the first chain of the multi-domain binding molecule of the invention may have the following format: N-TCEVL-TCEVH-VC1-C.

VC1 may comprise a TCRβ variable and constant region and VC2 may comprise a TCRα variable and constant region. Thus, VC1 and VC2 may dimerise to form a soluble TCR. In this regard, the second chain of the multi-domain binding molecule of the invention may have the following format: N-TCRβ-FC1-C (where “TCRβ” refers to the TCRβ variable and constant region and “TCRα” refers to the TCRα variable and constant region).

The TCE-VL region may be linked to the TCE-VH region via a sequence comprising a glycine-serine linker. Preferably, the sequence linking the TCE-VL region to the TCE-VH region is the amino acid sequence provided in SEQ ID NO: 46.

The TCE-VH region may be linked to VC1 via a sequence comprising, or consisting of, a glycine-serine linker. Preferably, the sequence linking the TCE-VH region to VC1 is the amino acid sequence provided in SEQ ID NO: 34.

VC2 may be linked to the FC1 region via a sequence comprising an IgG hinge sequence. The IgG hinge sequence may be at least 80% identical to SEQ ID NO: 33. Preferably, the IgG hinge sequence is at least 90%, at least 95%, at least 98% or is 100% identical to SEQ ID NO: 33.

The sequence linking VC2 to the FC1 region may further comprise a glycine-serine linker. Preferably, the glycine-serine linker has the sequence provided in SEQ ID NO: 42. Preferably, these sequences are in the following formats, N-terminal to C-terminal: VC2-GS linker-IgG hinge-FC1.

The FC1 region may be linked to VC2 via a sequence comprising a glycine-serine linker. Preferably, the glycine-serine linker linking the FC1 region VC2 region has the sequence provided in SEQ ID NO: 42.

The multi-domain binding molecule of the invention comprises three polypeptide chains. The multi-domain binding molecule may be soluble and/or recombinant and/or isolated. An exemplary multi-domain binding molecules may comprise the sequences of:
(a) SEQ ID NO: 52, SEQ ID NO: 54 and SEQ ID NO: 56
(b) SEQ ID NO: 52, SEQ ID NO: 58 and SEQ ID NO: 56; or
(c) SEQ ID NO: 52, SEQ ID NO: 60 and SEQ ID NO: 61.

The first chain of the multi-domain binding molecule may have an amino acid sequence that is at least 80% identical to the sequence of SEQ ID NO: 56. The first chain of the multi-domain binding molecule may have an amino acid sequence that is at least 90%, at least 95%, or at least 98% identical to the sequence of SEQ ID NO: 56. Preferably, the first chain of the multi-domain binding molecule comprises, or consists of, the amino acid sequence provided in SEQ ID NO: 56.

Alternatively, the first chain of the multi-domain binding molecule may have an amino acid sequence that is at least 80% identical to the sequence of SEQ ID NO: 61. The first chain of the multi-domain binding molecule may have an amino acid sequence that is at least 90%, at least 95%, or at least 98% identical to the sequence of SEQ ID NO: 61. For example, the first chain of the multi-domain binding molecule comprises, or consists of, the amino acid sequence provided in SEQ ID NO: 61.

The second chain of the multi-domain binding molecule may have an amino acid sequence that is at least 80% identical to the sequence of SEQ ID NO: 54. The second chain of the multi-domain binding molecule may have an amino acid sequence that is at least 90%, at least 95%, or at least 98% identical to the sequence of SEQ ID NO: 54. For example, the second chain of the multi-domain binding molecule comprises, or consists of, the amino acid sequence provided in SEQ ID NO: 54.

Alternatively, the second chain of the multi-domain binding molecule may have an amino acid sequence that is at least 80% identical to the sequence of SEQ ID NO: 58. The second chain of the multi-domain binding molecule may have an amino acid sequence that is at least 90%, at least 95%, or at least 98% identical to the sequence of SEQ ID NO: 58. For example, the second chain of the multi-domain binding molecule comprises, or consists of, the amino acid sequence provided in SEQ ID NO: 58.

Alternatively, the second chain of the multi-domain binding molecule may have an amino acid sequence that is at least 80% identical to the sequence of SEQ ID NO: 60. The second chain of the multi-domain binding molecule may have an amino acid sequence that is at least 90%, at least 95%, or at least 98% identical to the sequence of SEQ ID NO: 60. Preferably, the second chain of the multi-domain binding molecule comprises, or consists of, the amino acid sequence provided in SEQ ID NO: 60.

The third chain of the multi-domain binding molecule may have an amino acid sequence that is at least 80% identical to the sequence of SEQ ID NO: 52. The third chain of the multi-domain binding molecule may have an amino acid sequence that is at least 90%, at least 95%, or at least 98% identical to the sequence of SEQ ID NO: 52. Preferably, the third chain of the multi-domain binding molecule comprises, or consists of, the amino acid sequence provided in SEQ ID NO: 52. Optionally, the multi-domain binding molecule sequences above may be further fused to one or more other polypeptide sequences.

The sequences above relate to multi-domain binding molecules comprising TCR chains that bind to a GVYDGREHTV (SEQ ID NO: 1) HLA-A*02 complex. A person skilled in the art could adapt these sequences to another target by replacing the TCR chains in SEQ ID NO: 54, 56, 58, 60 or 61 with sequences of a different TCR of interest.

### Amino Acid Sequences

Within the scope of the invention are phenotypically silent variants of any molecule disclosed herein. As used herein the term “phenotypically silent variants” is understood to refer to a variant which incorporates one or more further amino acid changes, including substitutions, insertions and deletions, in addition to those set out above, which variant has a similar phenotype to the corresponding molecule without said change(s). For the purposes of this application, phenotype comprises binding affinity (Ko and/or binding half-life) and specificity. The phenotype for a soluble multi-domain binding molecule may include potency of immune activation and purification yield, in addition to binding affinity and specificity.

Phenotypically silent variants may contain one or more conservative substitutions and/or one or more tolerated substitutions. By tolerated substitutions it is meant those substitutions which do not fall under the definition of conservative as provided below but are nonetheless phenotypically silent. The skilled person is aware that various amino acids have similar properties and thus are ‘conservative’. One or more such amino acids of a protein, polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). It should be appreciated that amino acid substitutions within the scope of the present invention can be made using naturally occurring or non-naturally occurring amino acids. For example, it is contemplated herein that the methyl group on an alanine may be replaced with an ethyl group, and/or that minor changes may be made to the peptide backbone. Whether or not natural or synthetic amino acids are used, it is preferred that only L-amino acids are present.

Substitutions of this nature are often referred to as “conservative” or “semi-conservative” amino acid substitutions. The present invention therefore extends to use of a molecule comprising any of the amino acid sequences described above but with one or more conservative substitutions and or one or more tolerated substitutions in the sequence, such that the amino acid sequence of the molecule, or any domain or region thereof, has at least 90% identity, such as 90%, 91%, 92%, 93%, 94%, 95%, 96%, 97%, 98%, 99% or 100% identity, to the sequences disclosed herein.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The BLASTn and BLASTp programs of Altschul, et al. (1990) J. Mol. Biol. 215:403-410 have incorporated such an algorithm. Determination of percent identity between two nucleotide sequences can be performed with the BLASTn program. Determination of percent identity between two protein sequences can be performed with the BLASTp program. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., BLASTp and BLASTp) can be used. See http://www.ncbi.nlm.nih.gov. Default general parameters may include for example, Word Size=3, Expect Threshold=10. Parameters may be selected to automatically adjust for short input sequences. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search. For the purposes of evaluating percent identity in the present disclosure, BLASTp with the default parameters is used as the comparison methodology. In addition, when the recited percent identity provides a non-whole number value for amino acids (i.e., a sequence of 25 amino acids having 90% sequence identity provides a value of “22.5”, the obtained value is rounded down to the next whole number, thus “22”). Accordingly, in the example provided, a sequence having 22 matches out of 25 amino acids is within 90% sequence identity.

As will be obvious to those skilled in the art, it may be possible to truncate, or extend, the sequences provided at the C-terminus and/or N-terminus thereof, by 1, 2, 3, 4, 5 or more residues, without substantially affecting the functional characteristics of the molecule, for example a TCR portion. The sequences provided at the C-terminus and/or N-terminus thereof may be truncated or extended by 1, 2, 3, 4 or 5 residues. All such variants are encompassed by the present invention.

Mutations, including conservative and tolerated substitutions, insertions and deletions, may be introduced into the sequences provided using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning—A Laboratory Manual (3^{rd} Ed.) CSHL Press. Further information on ligation independent cloning (LIC) procedures can be found in Rashtchian, (1995) Curr Opin Biotechnol 6(1): 30-6. The protein sequences provided herein may be obtained from recombinant expression, solid state synthesis, or any other appropriate method known in the art.

### Assessing Binding Characteristics and Activity of Multi-Domain Binding Molecules

Methods to determine binding affinity (inversely proportional to the equilibrium constant Ko) and binding half-life (expressed as T½) are known to those skilled in the art. Binding affinity and binding half-life may be determined using Surface Plasmon Resonance (SPR) or Bio-Layer Interferometry (BLI), for example using a BIAcore instrument or Octet instrument, respectively. It will be appreciated that doubling the affinity results in halving the K_{D}. T½ is calculated as In2 divided by the off-rate (k_{off}).

Therefore, doubling of T½ results in a halving in k_{off}. K_{D} and k_{off} values for TCRs are usually measured for soluble forms of the TCR, i.e. those forms which are truncated to remove cytoplasmic and transmembrane domain residues. To account for variation between independent measurements, and particularly for interactions with dissociation times in excess of 20 hours, the binding affinity and or binding half-life of a given protein may be measured several times, for example 3 or more times, using the same assay protocol, and an average of the results taken. To compare binding data between two samples (i.e. two different proteins and or two preparations of the same protein) it is preferable that measurements are made using the same assay conditions (e.g. temperature). Measurement methods described in relation to TCRs may also be applied to the multi-domain binding molecules described herein.

Certain multi-domain binding molecules of the invention are able to generate a highly potent T cell response in vitro against antigen positive cells, in particular those cells presenting low levels of antigen typical of cancer cells (i.e. in the order of 5-100, for example 50, antigens per cell (Bossi et al., (2013) Oncoimmunol. 1; 2 (11):e26840; Purbhoo et al., (2006). J Immunol 176(12): 7308-7316.). The T cell response that is measured may be the release of T cell activation markers such as Interferon γ or Granzyme B, or target cell killing, or other measure of T cell activation, such as T cell proliferation. A highly potent response may be one with EC₅₀ value in the nM-pM range, for example 500 nM or lower, preferably 1 nM or lower, or 500 pM or lower.

Alternatively, certain binding molecules of the invention may generate a highly potent anti-inflammatory response, such as CD8+ cell killing and/or CD4+ inflammation inhibition. Such binding molecules may be in soluble form and linked to an immune suppressor such as a PD-1 agonist or an interleukin or cytokine such as IL-2, IL-4, IL-10 or IL-13. The anti-inflammatory response that is measured may be CD8+ cell killing and/or CD4+ inflammation inhibition, and or inhibition of CD8+ T cell signalling pathways. Suitable methods for assessing an anti-inflammatory response will be known in the art and include the Jurkat NFAT cell reporter assay described in Example 11. Preferably a highly potent response is one with IC₅₀ value in the pM range, i.e. 1000 pM or lower. Preferably the maximum inhibition obtained in reporter assays is greater than 50%, for example 80% or more.

Molecules encompassed by the present invention may have an improved half-life. Methods for determining whether a protein has an improved half-life will be apparent to the skilled person. For example, the ability of a protein to bind to a neonatal Fc receptor (FcRn) is assessed. In this regard, increased binding affinity for FcRn increases the serum half-life of the protein (see for example, Kim et al. Eur J Immunol., 24:2429, 1994).

The half-life of a protein of the disclosure can also be measured by pharmacokinetic studies, e.g., according to the method described by Kim et al. Eur J of Immunol 24: 542, 1994. According to this method radiolabeled protein is injected intravenously into mice and its plasma concentration is periodically measured as a function of time, for example at 3 minutes to 72 hours after the injection. Alternatively, unlabeled protein of the disclosure can be injected and its plasma concentration periodically measured using an ELISA. The clearance curve thus obtained should be biphasic, that is, an alpha phase and beta phase. For the determination of the in vivo half-life of the protein, the clearance rate in beta-phase is calculated and compared with that of the wild type or unmodified protein.

### Nucleic Acids, Vectors and Host Cells

The present invention provides a nucleic acid encoding a multi-domain binding molecule of the invention. The nucleic acid may be cDNA. The nucleic acid may be mRNA. The nucleic acid may be non-naturally occurring and/or purified and/or engineered. The nucleic acid sequence may be codon optimised, in accordance with the expression system utilised. As is known to those skilled in the art, expression systems may include bacterial cells such as E. coli, or yeast cells, or mammalian cells, or insect cells, or they may be cell free expression systems.

The present invention also provides constructs in the form of plasmids, vectors, transcription or expression cassettes which comprise at least one nucleic acid as described above. The present invention also provides a recombinant host cell which comprises one or more constructs as above. As mentioned, a nucleic acid encoding a specific binding molecule of the invention forms an aspect of the present invention, as does a method of production of the specific binding molecule comprising expression from a nucleic acid encoding a specific binding molecule of the invention. Expression may conveniently be achieved by culturing recombinant host cells containing the nucleic acid under appropriate conditions. Following production by expression, a specific binding molecule may be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli. The expression of antibodies and antibody fragments in prokaryotic cells such as E. coli is well established in the art. For a review, see for example Pluckthun, Bio/Technology 9:545-551 (1991). Expression in eukaryotic cells in culture is also available to those skilled in the art as an option for production of a specific binding molecule, see for recent review, for example Reff, Curr. Opinion Biotech. 4:573-576 (1993); Trill et al., Curr. Opinion Biotech. 6:553-560 (1995).

Suitable vectors can be chosen or constructed, containing appropriate regulatory sequences, including promoter sequences, terminator sequences, polyadenylation sequences, enhancer sequences, marker genes and other sequences as appropriate. Vectors may be any suitable vectors known in the art, including plasmids or viral vectors (e.g. ‘phage, or phagemid), as appropriate. For further details see, for example, Sambrook et al., Molecular Cloning: A Laboratory Manual: 2nd Edition, Cold Spring Harbor Laboratory Press (1989). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. eds., Short Protocols in Molecular Biology, 2nd Edition, John Wiley & Sons (1992).

The present invention also provides a host cell containing a nucleic acid as disclosed herein. Further, the invention provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene.

Suitable host cells for cloning or expression of polynucleotides and/or vectors of the present invention are known in the art. Suitable host cells for the expression of (glycosylated) proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR− CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for protein production, see, e.g., Yazaki, P. and Wu, A. M., Methods in Molecular Biology, Vol. 248, Lo, B. K. C. (ed.), Humana Press, Totowa, NJ (2004), pp. 255-268. The host cell may be eukaryotic, e.g., a Chinese Hamster Ovary (CHO) cell or lymphoid cell (e.g., Y0, NS0, Sp20 cell).

The nucleic acid of the invention may be integrated into the genome (e.g. chromosome) of the host cell. Integration may be promoted by inclusion of sequences which promote recombination with the genome, in accordance with standard techniques.

### Methods of Making Multi-Domain Binding Molecules

Further provided herein are methods for making the multi-domain binding molecule described herein. The methods comprise maintaining the host cell of the invention under optimal conditions for expression of the nucleic acid or expression vector of the invention and isolating the multi-domain binding molecule.

Methods of producing recombinant proteins are well known in the art. Nucleic acids encoding the protein can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary mammalian cells used for expressing a protein are CHO cells, myeloma cells or HEK cells. Molecular cloning techniques to achieve these ends are known in the art and described, for example in Ausubel et al. (editors), Current Protocols in Molecular Biology, Greene Pub. Associates and Wiley-Interscience (1988, including all updates until present) or Sambrook et al. Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

The nucleic acid may be inserted operably linked to a promoter in an expression construct or expression vector for further cloning (amplification of the DNA) or for expression in a cell-free system or in cells. As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid. As used herein, the term “operably linked to” means positioning a promoter relative to a nucleic acid such that expression of the nucleic acid is controlled by the promoter.

Many vectors for expression in cells are commercially available. The vector components generally include, but are not limited to, one or more of the following: a signal sequence, a sequence encoding a protein (e.g., derived from the information provided herein), an enhancer element, a promoter, and a transcription termination sequence. The skilled person will be aware of suitable sequences for expression of a protein. Exemplary signal sequences include prokaryotic secretion signals (e.g., pe1B, alkaline phosphatase, penicillinase, lpp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-a promoter (EF1), small nuclear RNA promoters (Ula and Ulb), a-myosin heavy chain promoter, Simian virus 40 promoter (SV40), Rous sarcoma virus promoter (RSV), Adenovirus major late promoter, b-actin promoter; hybrid regulatory element comprising a CMV enhancer/b-actin promoter or an immunoglobulin promoter or an active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GALA promoter, the CUP1 promoter, the PH05 promoter, the nmt promoter, the RPR1 promoter, or the TEF1 promoter.

The host cells used to produce the protein may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

Methods for isolating a protein are known in the art. Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing.

These methods are known in the art and described, for example in WO99/57134 or Ed Harlow and David Lane (editors) Antibodies: A Laboratory Manual, Cold Spring Harbor Laboratory, (1988). The skilled person will also be aware that a protein can be modified to include a tag to facilitate purification or detection, e.g., a poly-histidine tag, a hexa-histidine tag, a influenza virus hemagglutinin (HA) tag, a Simian Virus 5 (V5) tag, a LLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Molecules of the invention may be amenable to high yield purification. Yield may be determined based on the amount of material retained during the purification process (i.e. the amount of correctly folded material obtained at the end of the purification process relative to the amount of solubilised material obtained prior to refolding), and or yield may be based on the amount of correctly folded material obtained at the end of the purification process, relative to the original culture volume. High yield means greater than 1%, or greater than 5%, or higher yield. High yield means greater than 1 mg/ml, or greater than 3 mg/ml, or greater than 5 mg/ml, or higher yield.

### Pharmaceutical Compositions and Medical Methods

For administration to patients, the molecules of the invention, nucleic acids, expression vectors or cells of the invention may be provided as part of a pharmaceutical composition together with one or more pharmaceutically acceptable carriers or excipients. This pharmaceutical composition may be in any suitable form, (e.g. depending upon the desired method of administering it to a patient). It may be provided in unit dosage form, and will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, such as parenteral (including subcutaneous, intramuscular, intrathecal or intravenous), enteral (including oral or rectal), inhalation or intranasal routes. Such compositions may be prepared by any method known in the art of pharmacy, for example by mixing the active ingredient with the carrier(s) or excipient(s) under sterile conditions. Methods for preparing a protein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions will commonly comprise a solution of the multi-domain binding molecule of the invention (or the nucleic acid, cell, or vector of the invention) dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of molecules of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Molecules of the invention may have an ideal safety profile for use as therapeutic agents. An ideal safety profile means that in addition to demonstrating good specificity, the molecules of the invention may have passed further preclinical safety tests. Examples of such tests include whole blood assays to confirm minimal cytokine release in the presence of whole blood and thus low risk of causing a potential cytokine release syndrome in vivo, and alloreactivity tests to confirm low potential for recognition of alternative HLA types.

Binding molecules of the present invention may have an extended in vitro half-life while retaining a similar therapeutic window to an equivalent non-Fc molecule. The term “therapeutic window” as used herein refers to the difference between on-target activity and off-target activity, for example the difference between on-target T cell activation and off-target T cell activation.

Dosages of the molecules of the present invention can vary between wide limits, depending upon the disease or disorder to be treated, the age and condition of the individual to be treated, etc. A physician will ultimately determine appropriate dosages to be used.

Multi-domain binding molecules, pharmaceutical compositions, vectors, nucleic acids and cells of the invention may be provided in substantially pure form, for example, at least 80%, at least 85%, at least 90%, at least 91%, at least 92%, at least 93%, at least 94%, at least 95%, at least 96%, at least 97%, at least 98%, at least 99% or 100% pure.

The multi-domain binding molecule of the invention may be further associated with a therapeutic agent. Therapeutic agents which may be associated with the molecules of the invention include immune-modulators and effectors, radioactive compounds, enzymes (perforin for example) or chemotherapeutic agents (cis-platin for example). To ensure that toxic effects are exercised in the desired location the toxin could be inside a liposome linked to the multi-domain binding molecule described herein so that the compound is released slowly. This will prevent damaging effects during the transport in the body and ensure that the toxin has maximum effect after binding of the multi-domain binding molecule described herein to the relevant antigen presenting cells.

Examples of suitable therapeutic agents include, but are not limited to:
small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E, vincristine and doxorubicin;
peptide cytotoxins, i.e. proteins or fragments thereof with the ability to kill mammalian cells. For example, ricin, diphtheria toxin, pseudomonas bacterial exotoxin A, Dnase and Rnase;
radio-nuclides, i.e. unstable isotopes of elements which decay with the concurrent emission of one or more of α or β particles, or γ rays. For example, iodine 131, rhenium 186, indium 111, yttrium 90, bismuth 210 and 213, actinium 225 and astatine 213; chelating agents may be used to facilitate the association of these radio-nuclides to the multi-domain binding molecule;
Immuno-stimulants, i.e. immune effector molecules which stimulate immune response. For example, cytokines such as IL-2 and IFN-γ,
Superantigens and mutants thereof;
TCR-HLA fusions, e.g. fusion to a peptide-HLA complex, wherein said peptide is derived from a common human pathogen, such as Epstein Barr Virus (EBV);
chemokines such as IL-8, platelet factor 4, melanoma growth stimulatory protein, etc.;
antibodies or fragments thereof, including anti-T cell or NK cell determinant antibodies (e.g. anti-CD3, anti-CD28 or anti-CD16);
antibodies or fragments thereof that bind to molecules that locate to the immune synapse
alternative protein scaffolds with antibody like binding characteristics
complement activators;
xenogeneic protein domains, allogeneic protein domains, viral/bacterial protein domains, viral/bacterial peptides.

The multi-domain binding molecule, nucleic acid, vector, pharmaceutical composition and cell of the invention may be used for treating diseases such as cancer, particularly cancers which are associated with expression of a tumour-associated antigen. For example, the cancer may be associated with expression of GP100, NYESO, MAGEA4, or PRAME as described in WO2011001152, WO2017109496, WO2017175006 and WO2018234319, and, for example, in corresponding U.S. Pat. Nos. 8,519,100, 11,639,374, 11,505,590, and 11,427,624, the contents of each which are herein incorporated by reference.

The cancer to be treated may be a cancer associated with MAGEA4 expression. By “associated with MAGEA4 expression” it is meant that the cancer comprises cancer cells that express MAGEA4. In this regard, the cancer may be MAGEA4-positive cancer. The cancer may be known to be associated with expression of MAGEA4, and thus MAGEA4 expression may not be assessed. Alternatively, MAGEA4 expression can be assessed using any method known in the art, including, for example, histological methods. However, the invention is not intended to be limited to the treatment of cancers for which MAGEA4 expression can be detected by histological methods. Cancers associated with MAGEA4 expression include, but are not limited to, ovarian cancer, lung cancer, head and neck cancer, oesophageal cancer, breast cancer, synovial sarcoma, gastric cancer, bladder cancer, and any tumour with squamous cell histology. The head and neck cancer may be head and neck squamous cell carcinoma (HNSCC). The lung cancer may be non-small cell lung carcinoma (NSCLC). The bladder cancer may be urothelial carcinoma. The oesophageal cancer may be gastroesophageal junction (GEJ) adenocarcinoma. The ovarian cancer may be epithelial ovarian cancer, such as high grade serous ovarian cancer.

The multi-domain binding molecule, nucleic acid, vector, pharmaceutical composition and cell of the invention may be used for treating an infectious disease. The infectious disease may be caused by a bacterial, viral, fungal or parasitic pathogen. Any infection with a pathogen which results in antigen-presenting cells presenting MHC bound to a peptide from the pathogen may be suitable for treatment with the multi-domain binding molecule of the invention. The multi-domain binding molecule of the invention is particularly well suited to infections where antigen-presenting cells present levels of pathogen peptide that are lower than optimal for the natural immune system to clear the infection without additional treatment. The infectious disease may be a chronic infection. Exemplary infectious diseases include Hepatitis B virus (HBV) infection and human immunodeficiency virus (HIV) infection.

The multi-domain binding molecule of the invention may be used in a method of treating an autoimmune disease, such as type 1 diabetes. Organ-specific immune suppression, rather than systemic immunosuppression, may be a beneficial route for treatment given the potential significant adverse events associated with systemic immunosuppression. In autoimmunity, there is also mounting evidence that PD-1 pathway impairment plays an important role in disease pathogenesis. PD-1, PD-L1 and PD-L2 gene polymorphisms are associated with several autoimmune diseases. Abnormally low PD-L1 expression has been observed in samples from type 1 diabetes and Crohn's disease patients. Activating PD-1 on autoreactive lymphocytes thus may serve as a mechanism to treat autoimmune diseases. Effective therapeutics for the treatment of autoimmune diseases include those having an advantageous risk profile (e.g., a high level of target and tissue specificity) and are capable of being administered with less frequency.

Also provided by the invention are:
A multi-domain binding molecule, nucleic acid, vector, pharmaceutical composition or cell of the invention for use in medicine, preferably for use in a method of treating cancer or a tumour or an autoimmune or infectious disease;
the use of a multi-domain binding molecule, nucleic acid, vector, pharmaceutical composition or cell of the invention in the manufacture of a medicament for treating cancer or a tumour or an autoimmune disease or infectious disease;
a method of treating cancer or a tumour or an autoimmune disease or infectious disease in a patient, comprising administering to the patient a multi-domain binding molecule, nucleic acid, vector, pharmaceutical composition or cell of the invention;
an injectable formulation for administering to a human subject comprising a multi-domain binding molecule, nucleic acid, vector pharmaceutical composition or cell of the invention.

The method of treatment may further include administering separately, in combination, or sequentially, an additional anti-neoplastic agent. Example of such agents are known in the art and may include immune activating agents and/or T cell modulating agents.

### Kits and Articles of Manufacture

In another aspect, a kit or an article of manufacture containing materials useful for the treatment and/or prevention of the diseases described above is provided.

The kit may comprise (a) a container comprising the molecule, nucleic acid, vector or cell of the invention, optionally in a pharmaceutically acceptable carrier or diluent; and (b) a package insert with instructions for treating a disease (e.g., cancer, autoimmune disease or an infectious disease) in a subject. The kit may further comprise (c) at least one further therapeutically active compound or drug.

The package insert may be on or associated with the container. Suitable containers include, for example, bottles, vials, syringes, etc. The containers may be formed from a variety of materials such as glass or plastic. The container holds or contains a composition that comprises the molecule, nucleic acid, vector or cell of the invention and may have a sterile access port (for example, the container may be an intravenous solution bag or a vial having a stopper pierceable by a hypodermic injection needle). At least one active agent in the composition is the molecule, nucleic acid, vector or cell of the invention. The label or package insert indicates that the composition is used for treating a subject eligible for treatment, e.g., one having or predisposed to developing a disease described herein, with specific guidance regarding dosing amounts and intervals of the composition and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The kit optionally further comprises a container comprising a second medicament, wherein the molecule, nucleic acid, vector or cell of the invention is a first medicament, and which kit further comprises instructions on the package insert for treating the subject with the second medicament, in an effective amount.

Various modifications of the invention in addition to those shown and described herein will become apparent to those skilled in the art from the foregoing description and fall within the scope of the appended claims. Preferred features of each aspect of the invention are as for each of the other aspects mutatis mutandis. The documents referred to herein are incorporated by reference to the fullest extent permitted by law.

### DESCRIPTION OF THE DRAWINGS

FIG. 1A is a schematic diagram of a T-Cell Receptor (TCR):anti-CD3 fusion molecule according to the present invention: (i) shows the components of the three individual chains; (ii) shows the overall structure of the molecule. This format is defined as Mol020 herein.

FIG. 1B is a schematic diagram illustrating alternative TCR:anti-CD3 fusion molecule formats used in the examples: (i) is a two-chain molecule fused to Fc, this format is defined as Mol014 herein; (ii) is a fusion molecule lacking an Fc domain, this format is defined as Mol001 herein.

FIG. 2 shows a comparison of T cell activation, defined as IFNγ release, in the presence and absence of antigen positive cells for Mol020 and Mol014. In this example the TCR portion of the molecule binds to a peptide from MAGEA4. T2 cells pulsed with MAGEA4 peptide or an irrelevant peptide were used as antigen positive and antigen negative cells respectively. Mol020 gave rise to a substantially more potent T cell response compared to Mol014, whilst retaining a high degree of specificity.

FIG. 3 shows a further comparison of T cell activation, defined as IFNγ release, in the presence and absence of antigen positive cells for Mol020 and Mol014. In this example the TCR portion of the molecule recognises a peptide from PRAME. Human Melanoma Mel624 cells were used as antigen positive cells. Mel624 with MAGEA4 gene knock out were used as antigen negative cells. Mol020 was substantially more potent than Mol014, while retaining a high degree of specificity.

FIG. 4 shows a comparison of T cell activation, defined as IFNγ release, in the presence and absence of antigen positive cells for Mol020 and Mol001 molecules targeting MAGEA4. Human lung cancer NCI-H1755 cells were used as antigen positive targets cells. Human melanoma Mel202 cells which had been prior transduced with HLA-A2 B2M were used as antigen negative cells. Mol020 showed lower T cell activation against both antigen positive and antigen negative cells. Therefore, the therapeutic window for Mol020, relative to the non-Fc fusion Mol001, is maintained.

FIG. 5 shows pharmacokinetic (PK) assessment of Mol020 and Mol001 in Tg32 SCID mice. Comparison of serum concentration over time demonstrated a substantially extended in vivo half life for Mol020 relative to Mol001.

FIG. 6 provides a schematic diagram showing each of the glycosylation site variants, with the locations of the glycosylation sites indicated. The three glycosylation sites in the TCR variable domains are shown at positions N18 and N24 of the alpha chain and N84 of the beta chain. The corresponding relative yield is shown below each of the variants, the melting temperature (Tm) of the TCR portion of the molecule is also shown. The data show that removing all TCR 7 glycosylation sites negatively impacted yield and molecule stability. Retaining a single glycosylation site at position N18 of the alpha chain partially compensates for these effects.

FIG. 7 shows stability overtime of monoglycosylated (Mol020v14) and aglycosylated (Mol020v13) molecules in human serum. Dotted lines indicate 20% change from baseline. The data show that the monoglycosylated variant (v14) demonstrated improved stability in serum relative to the aglycosylated variant (v13).

FIG. 8 shows pharmacokinetic (PK) assessment of monoglycosylated (mol020v14) and aglycosylated (mol020v13) variants in Tg32 SCID mice. A fully glycosylated variant, retaining all 7 glycosylation sites intact, was used as a control.

FIG. 9 shows T cell activation, defined as IFNγ release, for monoglycosylated (mol020v14) and aglycosylated (mol020v13) variants, in the presence or absence of antigen positive and antigen negative primary cell lines. The data indicated that both molecules drive potent T cell activation in the presence of antigen positive cancer cells

FIG. 10: Schematic of an exemplary TCR-PD-1 agonist binding molecule incorporating an Fc domain

FIG. 11: a) Graphs showing inhibition of T cell signalling in Jurkat NFAT reporter assay by TCR PD-1 agonist binding molecules b) A graph comparing inhibition of T cell signalling in Jurkat NFAT reporter assay by TCR PD-1 agonist with and without Fc.

FIG. 12: A graph showing in vivo concentration of TCR PD-1 agonist binding molecule in SCID mice over 3 weeks following intravenous (IV) or subcutaneous (SC) administration.

FIG. 13: A graph showing inhibition of IL2 release by primary CD4+ T cells in the presence of TCR PD-1 agonist

FIG. 14: Graphs showing inhibition of B cell killing (a) and IFNγ cytokine release (b) by two autoreactive T cell clones in the presence of TCR PD-1 agonist binding molecule FIG. 15: Graphs showing inhibition of stimulation in PD1 +ve and PD-1 −ve NK cells indicated by % cells positive for CD107a and IFNγ, in the presence of TCR PD-1 agonist binding molecules (*p≤0.05, **p≤0.01; ns=not significant).

### DESCRIPTION OF THE SEQUENCES

SEQ ID NO: 1 HLA-A*02 restricted peptide:
GVYDGREHTV

SEQ ID NO: 5 Amino acid sequence of the TCRα chain variable domain of an exemplary TCR. CDRs (CDR1, CDR2 and CDR3) are underlined and are designated SEQ ID NO: 2, 3 and 4 respectively, framework regions (FR1, FR2, FR3 and FR4) are in italics and are designated SEQ ID NO: 13, 16, 17 and 18 respectively. This sequence contains a N24Q mutation (double underlined).

ANQVEQSPQSLIILEGKNVTLQCQYTVSPFSNLRWYKQDTGRGPVSLTI
LTFSENTKSNGRYTATLDADTKQSSLHITASQLSDSASYICVVNSAQGL
YIPTFGRGTSLIVHP

SEQ ID NO: 11 Amino acid sequence of the TCRβ chain variable domain of an exemplary TCR. CDRs (CDR1, CDR2 and CDR3) are underlined and are designated SEQ ID NO: 8, 9 and 10 respectively, framework regions (FR1, FR2, FR3 and FR4) are in italics and are designated SEQ ID NO: 19, 20, 21 and 23 respectively. This sequence contains a N84Q mutation (double underlined).

DVKVTQSSRYLVKRTGEKVFLECVQDLDHENMFWYRQDPGLGLRLIYFS
RFATGKEKGDIPEGYSVSREKKERFSLILESASTQQTSMYLCASSSDQN
SGDPYEQYFGPGTRLTVT

SEQ ID NO: 24 Amino acid sequence of the TCRα chain of an exemplary TCR. CDRs (CDR1, CDR2 and CDR3) are underlined and are designated SEQ ID NO: 2, 3 and 4 respectively, framework regions (FR1, FR2, FR3 and FR4) are in italics and are designated SEQ ID NO: 13, 16, 17 and 18 respectively. The constant region is shown in bold and is designated SEQ ID NO: 29. Within the constant region, a non-native cysteine residue is double underlined (at position 48 of the constant region) which was introduced to create an inter-chain disulphide bond. The sequence also contains N24Q, N146Q, N180Q and N191Q substitutions (double underlined).

ANQVEQSPQSLIILEGKNVTLQC [symbol] YTVSPFSNLRWYKQDTGRGPVSLTILTFSENTKSNGRY
TATLDADTKQSSLHITASQLSDSASYICVVNSAQGLYIPTFGRGTSLIVHPYIQKPDPAVYQLR
DSKSSDKSVCLFTDFDSQT [symbol] VSQSKDSDVYITDK [symbol] VLDMRSMDFKSNSAVAWS [symbol] KSDFAC
ANAF [symbol] NSIIPEDT

SEQ ID NO: 27 Amino acid sequence of the TCRβ chain of an exemplary TCR. CDRs (CDR1, CDR2 and CDR3) are underlined and are designated SEQ ID NO: 8, 9 and 10 respectively, framework regions (FR1, FR2, FR3 and FR4) are in italics and are designated SEQ ID NO: 19, 20, 21 and 23 respectively. Constant region is shown in bold and is designated SEQ ID NO: 31. Within the constant region, a non-native cysteine residue is double underlined (at position 57 of the constant region) which was introduced to create an inter-chain disulphide bond. The sequence also contains N84Q and N186Q substitutions (double underlined).

DVKVTQSSRYLVKRTGEKVFLECVQDLDHENMFWYRQDPGLGLRLIYFSRFATGKEKGDIP
EGYSVSREKKERFSLILESASTQQTSMYLCASSSDQNSGDPYEQYFGPGTRLTVTEDLKNV
FPPEVAVFEPSEAEISHTQKATLVCLATGFYPDHVELSWWVNGKEVHSGV [symbol] TDPQPLKEQ
PAL [symbol] DSRYALSSRLRVSATFWQDPRNHFRCQVQFYGLSENDEWTQDRAKPVTQIVSAEA
WGRAD

SEQ ID NO: 62 An exemplary anti-CD3 scFv (immune cell engaging domain (ICE)) referred to herein as “UO”. The light chain variable domain (VL) is in italics and is designated SEQ ID NO: 66. The light chain CDRs (CDR1, CDR2 and CDR3) are underlined and are designated SEQ ID NO: 68, 69 and 70. The heavy chain variable domain (VH) is shown in bold and is designated SEQ ID NO: 74. The heavy chain CDRs (CDR1, CDR2 and CDR3) are underlined and are designated SEQ ID NO: 75, 72 and 73. A glycine-serine linker, linking the VL and VH, is shown in plain text and is designated SEQ ID NO: 46.

AIQMTQSPSSLSASVGDRVTITCRASQDIRNYLNWYQQKPGKAPKLLIYYTSRLESGVPSRFS
GSGSGTDYTLTISSLQPEDFATYYCQQGNTLPWTFGQGTKVEIKGGGGSGGGGSGGGGSG
GGGSGGGSEVQLVESGGGLVQPGGSLRLSCAASGYSFTGYTMNWVRQAPGKGLEWVAL
INPYKGVSTYNQKFKDRFTISVDKSKNTAYLQMNSLRAEDTAVYYCARSGYYGDSDWYFD
VWGQGTLVTVSS

SEQ ID NO: 63 Another exemplary anti-CD3 scFv (immune cell engaging domain (ICE)) referred to herein as “U28”. This sequence is the same as SEQ ID NO: 62 above, except for two substitutions double underlined (T164A and 1201F). The light chain variable domain (VL) is in italics and is designated SEQ ID NO: 66. The light chain CDRs (CDR1, CDR2 and CDR3) are underlined and are designated SEQ ID NO: 68, 69 and 70. The heavy chain variable domain (VH) is shown in bold and is designated SEQ ID NO: 67. The heavy chain CDRs (CDR1, CDR2 and CDR3) are underlined and are designated SEQ ID NO: 71, 72 and 73. A glycine-serine linker, linking the VL and VH, is shown in plain text and is designated SEQ ID NO: 46.

AIQMTQSPSSLSASVGDRVTITCRASQDIRNYLNWYQQKPGKAPKLLIYYTSRLESGVPSRFS
GSGSGTDYTLTISSLQPEDFATYYCQQGNTLPWTFGQGTKVEIKGGGGSGGGGSGGGGSG
GGGSGGGSEVQLVESGGGLVQPGGSLRLSCAASGYSFTGY [symbol] MNWVRQAPGKGLEWVAL
INPYKGVSTYNQKFKDRFT [symbol] SVDKSKNTAYLQMNSLRAEDTAVYYCARSGYYGDSDWYFD
VWGQGTLVTVSS

SEQ ID NO: 51 Human IgG1 Fc region (CH2 and CH3 domains), unmodified

APELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSR
WQQGNVFSCSVMHEALHNHYTQKSLSLSPGK

SEQ ID NO: 49 Another exemplary IgG1 Fc region sequence. This sequence has two substitutions, double underlined, relative to the above unmodified IgG1 Fc sequence (SEQ ID NO: 51). These are an N297G substitution for inhibiting binding to FcγR as well as a T366W substitution (knob-forming substitution) for enhancing dimerization with another Fc region (e.g., SEQ ID NO: 50) containing T366S, L368A, and Y407V substitutions (hole-forming substitutions). The numbering of the substitutions in this sequence is according to the EU numbering scheme.

APELLGGPSVFLFPPKPKDTLMISRTPEVTCVVVDVSHEDPEVKFNWYVDGVEVHNAKTKPR
EEQYGSTYRVVSVLTVLHQDWLNGKEYKCKVSNKALPAPIEKTISKAKGQPREPQVYTLPPS
RDELTKNQVSLWCLVKGFYPSDIAVEWESNGQPENNYKTTPPVLDSDGSFFLYSKLTVDKS
RWQQGNVFSCSVMHEALHNHYTQKSLSLSPGK

SEQ ID NO: 50 An exemplary IgG1 Fc region sequence. This sequence has four substitutions, (double underlined), relative to the above unmodified IgG1 Fc sequence (SEQ ID NO: 51). These are an N297G substitution for inhibiting binding to FcγR as well as T366S, L368A, and Y407V substitutions (hole-forming substitutions) for enhancing dimerization with another Fc region (e.g., SEQ ID NO: 49) containing a T366W substitution (knob-forming substitution). The numbering of the substitutions in this sequence is according to the EU numbering scheme.

APELLGGPSVFLFPPKPKDTLMISRTPEVTCVVVDVSHEDPEVKFNWYVDGVEVHNAKTKPR
EEQYGSTYRVVSVLTVLHQDWLNGKEYKCKVSNKALPAPIEKTISKAKGQPREPQVYTLPPS
RDELTKNQVSLSCAVKGFYPSDIAVEWESNGQPENNYKTTPPVLDSDGSFFLVSKLTVDKSR
WQQGNVFSCSVMHEALHNHYTQKSLSLSPGK

SEQ ID NO: 76 An exemplary IgG1 Fc region sequence. This sequence has seven substitutions, (double underlined), relative to the above unmodified IgG1 Fc sequence (SEQ ID NO: 51). These are an N297G substitution for inhibiting binding to FcγR, T366S, L368A, and Y407V substitutions (hole-forming substitutions) for enhancing dimerization with another Fc region (e.g., SEQ ID NO: 77) containing a T366W substitution (knob-forming substitution), and the substitutions M252Y, S254T and T256E, to increase binding to FcRn. The numbering of the substitutions in this sequence is according to the EU numbering scheme.

APELLGGPSVFLFPPKPKDTLYITREPEVTCVVVDVSHEDPEVKFNWYVDGVEVHNAKTKPR
EEQYGSTYRVVSVLTVLHQDWLNGKEYKCKVSNKALPAPIEKTISKAKGQPREPQVYTLPPS
RDELTKNQVSLSCAVKGFYPSDIAVEWESNGQPENNYKTTPPVLDSDGSFFLVSKLTVDKSR
WQQGNVFSCSVMHEALHNHYTQKSLSLSPGK

SEQ ID NO: 77 A further exemplary IgG1 Fc region sequence. This sequence has five substitutions, double underlined, relative to the above unmodified IgG1 Fc sequence (SEQ ID NO: 51). These are an N297G substitution for inhibiting binding to FcγR, a T366W substitution (knob-forming substitution) for enhancing dimerization with another Fc region (e.g., SEQ ID NO: 76) containing T366S, L368A, and Y407V substitutions (hole-forming substitutions), and the substitutions M252Y, S254T and T256E, to increase binding to FcRn.

The numbering of the substitutions in this sequence is according to the EU numbering scheme.

APELLGGPSVFLFPPKPKDTLYITREPEVTCVVVDVSHEDPEVKFNWYVDGVEVHNAKTKPR
EEQYGSTYRVVSVLTVLHQDWLNGKEYKCKVSNKALPAPIEKTISKAKGQPREPQVYTLPPS
RDELTKNQVSLWCLVKGFYPSDIAVEWESNGQPENNYKTTPPVLDSDGSFFLYSKLTVDKS
RWQQGNVFSCSVMHEALHNHYTQKSLSLSPGK

SEQ ID NO: 47 An exemplary IgG1 hinge sequence (containing a C to S substitution at position 5, numbered according to SEQ ID NO: 47, relative to the native human IgG1 sequence; double underlined):

EPKSSDKTHTCPPCP
SEQ ID NO: 33 A truncated IgG1 hinge sequence:
DKTHTCPPCP
SEQ ID NO: 48 An lgG4 hinge sequence:
ESKYGPPCPSCP

SEQ ID NO: 52 A complete amino acid sequence of the third polypeptide chain of an exemplary multi-domain binding molecule. The polypeptide comprises a hIgG1 Fc chain with a truncated hinge (underlined). The Fc region sequence corresponds to the Fc sequence in SEQ ID NO: 49 (italics), which in this case is the FC2 region, comprising N297G (effector attenuation) and T366W (knob forming substitutions) amino acid substitutions (double underline). The numbering of the substitutions in this sequence is according to the EU numbering scheme.

DKTHTCPPCPAPELLGGPSVFLFPPKPKDTLMISRTPEVTCVVVDVSHEDPEVKFNWYVDGV
EVHNAKTKPREEQYGSTYRVVSVLTVLHQDWLNGKEYKCKVSNKALPAPIEKTISKAKGQPR
EPQVYTLPPSRDELTKNQVSLWCLVKGFYPSDIAVEWESNGQPENNYKTTPPVLDSDGSFF
LYSKLTVDKSRWQQGNVFSCSVMHEALHNHYTQKSLSLSPGK

SEQ ID NO: 54 A complete amino acid sequence of the second polypeptide chain of an exemplary multi-domain binding molecule. The polypeptide comprises a TCRα chain, which in this case is VC2 provided in SEQ ID NO: 25 (bold text), linked via IgG1 Fc truncated hinge (underlined) to an Fc region sequence (plain text), which in this case is the FC1 region as provided in SEQ ID NO: 50, comprising the amino acid substitutions N297G (effector attenuation; double underlined) and T366S, L368A, Y407V (hole-forming substitutions; double underlined). The TCRα chain sequence also contains N18Q, N24Q, N146Q, N180Q and N191Q substitutions (double underlined) relative to the TCRα chain of SEQ ID NO: 26.

ANQVEQSPQSLIILEGK [symbol] VTLQC [symbol] YTVSPFSNLRWYKQDTGRGPVSLTILTFSENTKSNGR
YTATLDADTKQSSLHITASQLSDSASYICVVNSAQGLYIPTFGRGTSLIVHPYIQKPDPAVYQ
LRDSKSSDKSVCLFTDFDSQT [symbol] VSQSKDSDVYITDKCVLDMRSMDFKSNSAVAWS [symbol] KSDF
ACANAF [symbol] NSIIPEDTDKTHTCPPCPAPELLGGPSVFLFPPKPKDTLMISRTPEVTCVVVDVSH
EDPEVKFNWYVDGVEVHNAKTKPREEQYGSTYRVVSVLTVLHQDWLNGKEYKCKVSNKAL
PAPIEKTISKAKGQPREPQVYTLPPSRDELTKNQVSLSCAVKGFYPSDIAVEWESNGQPENN
YKTTPPVLDSDGSFFLVSKLTVDKSRWQQGNVFSCSVMHEALHNHYTQKSLSLSPGK

SEQ ID NO: 58 An alternative complete amino acid sequence of the second polypeptide chain of an exemplary multi-domain binding molecule. The polypeptide comprises a TCRα chain, which in this case is “VC2” provided in SEQ ID NO: 24, (bold text) linked via IgG1 Fc truncated hinge (underlined) to an Fc region sequence (plain text), which in this case is the FC1 region as provided in SEQ ID NO: 50, comprising the amino acid substitutions N297G (effector attenuation; double underlined) and T366S, L368A, Y407V (hole-forming substitutions; double underlined). The TCRα chain sequence also contains N24Q, N146Q, N180Q and N191Q substitutions (double underlined) relative to the TCRα chain of SEQ ID NO: 26. The N-linked glycosylation site at N18 (relative to SEQ ID NO: 26) has been retained in this polypeptide.

ANQVEQSPQSLIILEGKNVTLQC [symbol] YTVSPFSNLRWYKQDTGRGPVSLTILTFSENTKSNGR
YTATLDADTKQSSLHITASQLSDSASYICVVNSAQGLYIPTFGRGTSLIVHPYIQKPDPAVYQ
LRDSKSSDKSVCLFTDFDSQT [symbol] VSQSKDSDVYITDKCVLDMRSMDFKSNSAVAWSQKSDF
ACANAFQNSIIPEDTDKTHTCPPCPAPELLGGPSVFLFPPKPKDTLMISRTPEVTCVVVDVSH
EDPEVKFNWYVDGVEVHNAKTKPREEQY [symbol] STYRVVSVLTVLHQDWLNGKEYKCKVSNKAL
PAPIEKTISKAKGQPREPQVYTLPPSRDELTKNQVSLSCSVKGFYPSDIAVEWESNGQPENN
YKTTPPVLDSDGSFFLVSKLTVDKSRWQQGNVFSCSVMHEALHNHYTQKSLSLSPGK

SEQ ID NO: 60 A further alternative complete amino acid sequence of the second polypeptide chain of an exemplary multi-domain binding molecule. The polypeptide comprises a TCRα chain, which in this case is “VC2” provided in SEQ ID NO: 26 (bold text), linked via IgG1 Fc truncated hinge (underlined) to an Fc region sequence (plain text), which in this case is the FC1 region as provided in SEQ ID NO: 50, comprising the amino acid substitutions N297G (effector attenuation; double underlined) and T366S, L368A, Y407V (hole-forming substitutions; double underlined).

ANQVEQSPQSLIILEGKNVTLQCNYTVSPFSNLRWYKQDTGRGPVSLTILTFSENTKSNGRY
TATLDADTKQSSLHITASQLSDSASYICVVNSAQGLYIPTFGRGTSLIVHPYIQKPDPAVYQL
RDSKSSDKSVCLFTDFDSQTNVSQSKDSDVYITDKCVLDMRSMDFKSNSAVAWSNKSDFA
CANAFNNSIIPEDTDKTHTCPPCPAPELLGGPSVFLFPPKPKDTLMISRTPEVTCVVVDVSHE
DPEVKFNWYVDGVEVHNAKTKPREEQYGSTYRVVSVLTVLHQDWLNGKEYKCKVSNKALP
APIEKTISKAKGQPREPQVYTLPPSRDELTKNQVSLSCAVKGFYPSDIAVEWESNGQPENNY
KTTPPVLDSDGSFFLVSKLTVDKSRWQQGNVFSCSVMHEALHNHYTQKSLSLSPGK

SEQ ID NO: 56: The complete amino acid sequence of the first polypeptide chain of an exemplary multi-domain binding molecule. The polypeptide comprises a immune cell engaging domain (ICE) (dash underlined) comprising the anti-CD3 scFv sequence provided in SEQ ID NO: 63 (“U28”). The VH (SEQ ID NO: 67; dash underlined, plain text) and VL (SEQ ID NO: 66; dash underlined, italics) of the scFv are linked by the glycine-serine linker of SEQ ID NO: 46 (dash underlined, bold text). The scFv is linked to a pMHC binding domain (not underlined) comprising the TCRβ chain sequence (which in this case is “VC1”) provided in SEQ ID NO: 27 with the glycine-serine linker of SEQ ID NO: 34 (underlined, bold text). The TCRβ variable domain is shown in italics and is designated SEQ ID NO: 11. The TCRβ constant domain is shown in bold and is designated SEQ ID NO: 31

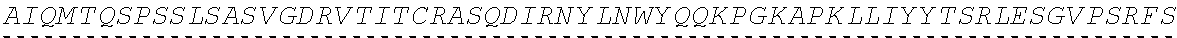

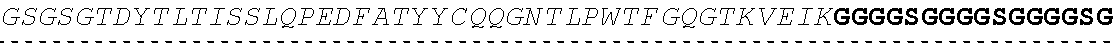

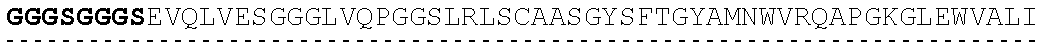

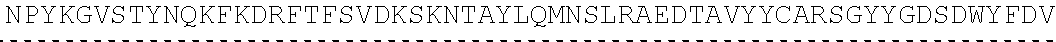

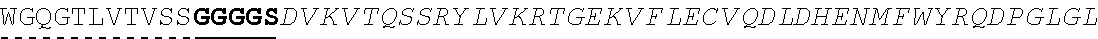

RLIYFSRFATGKEKGDIPEGYSVSREKKERFSLILESASTQQTSMYLCASSSDQNSGDPYEQ
YFGPGTRLTVTEDLKNVFPPEVAVFEPSEAEISHTQKATLVCLATGFYPDHVELSWWVNGK
EVHSGVCTDPQPLKEQPALQDSRYALSSRLRVSATFWQDPRNHFRCQVQFYGLSENDEW
TQDRAKPVTQIVSAEAWGRAD

SEQ ID NO: 61: The complete amino acid sequence of the first polypeptide chain of an alternative exemplary multi-domain binding molecule. The polypeptide comprises a immune cell engaging domain (ICE) (dash underlined) comprising the anti-CD3 scFv sequence provided in SEQ ID NO: 63 (“U28”). The VH (SEQ ID NO: 67; dash underlined, plain text) and VL (SEQ ID NO: 66; dash underlined, italics) of the scFv are linked by the glycine-serine linker of SEQ ID NO: 46 (dash underlined, bold text). The scFv is linked to a pMHC binding domain (not underlined) comprising the TCRβ chain sequence (which in this case is “VC1”) provided in SEQ ID NO: 28 with the glycine-serine linker of SEQ ID NO: 34 (underlined, bold text). The TCRβ variable domain is shown in italics and is designated SEQ ID NO: 12. The TCRβ constant domain is shown in bold and is designated SEQ ID NO: 32.

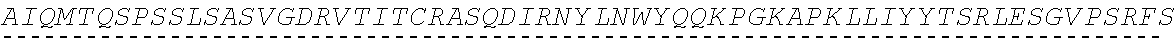

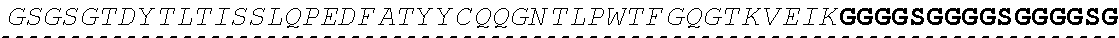

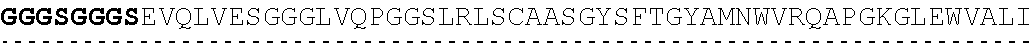

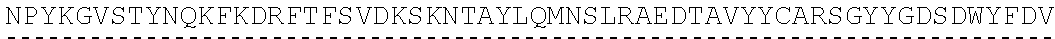

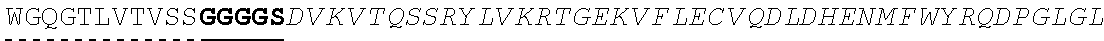

RLIYFSRFATGKEKGDIPEGYSVSREKKERFSLILESASTNQTSMYLCASSSDQNSGDPYEQ
YFGPGTRLTVTEDLKNVFPPEVAVFEPSEAEISHTQKATLVCLATGFYPDHVELSWWVNGK
EVHSGVCTDPQPLKEQPALNDSRYALSSRLRVSATFWQDPRNHFRCQVQFYGLSENDEW
TQDRAKPVTQIVSAEAWGRAD

### Additional Linker Sequences:

(SEQ ID NO: 34)
GGGGS,
(SEQ ID NO: 35)
GGGSG,
(SEQ ID NO: 36)
GGSGG,
(SEQ ID NO: 37)
GSGGG,
(SEQ ID NO: 38)
GSGGGP,
(SEQ ID NO: 39)
GGEPS,
(SEQ ID NO: 40)
GGEGGGP,
(SEQ ID NO: 41)
GGEGGGSEGGGS,
(SEQ ID NO: 42)
GGGSGGGG,
(SEQ ID NO: 43)
GGGGSGGGGSGGGGSGGGGS,
(SEQ ID NO: 44)
EAAAK,
(SEQ ID NO: 45)
EAAAKEAAAKEAAAK,
and
(SEQ ID NO: 46)
GGGGSGGGGSGGGGSGGGGSGGGS.

TABLE 1

| SEQ ID | | |
|---|---|---|
| NO: | Description | Sequence |
| 1 | MAGEA4 HLA-A*02 | GVYDGREHTV |
| | restricted peptide | |
| 2 | MAGEA4 specific TCR | VSPFSN |
| | alpha CDR1 | |
| 3 | MAGEA4 specific TCR | LTFSENT |
| | alpha CDR2 | |
| 4 | MAGEA4 specific TCR | VVNSAQGLYIPTF |
| | alpha CDR3 | |
| 5 | MAGEA4 specific TCR | ANQVEQSPQSLIILEGKNVTLQCQYTVSPFSNLRWYKQDTGRGPV |
| | alpha chain variable | SLTILTFSENTKSNGRYTATLDADTKQSSLHITASQLSDSASYICVV |
| | domain | NSAQGLYIPTFGRGTSLIVHP |
| | (monoglycosylated | |
| | form) | |
| 6 | MAGEA4 specific TCR | ANQVEQSPQSLIILEGKQVTLQCQYTVSPFSNLRWYKQDTGRGPV |
| | alpha chain variable | SLTILTFSENTKSNGRYTATLDADTKQSSLHITASQLSDSASYICVV |
| | domain (aglycosylated | NSAQGLYIPTFGRGTSLIVHP |
| | form) | |
| 7 | MAGEA4 specific TCR | ANQVEQSPQSLIILEGKNVTLQCNYTVSPFSNLRWYKQDTGRGPV |
| | alpha chain variable | SLTILTFSENTKSNGRYTATLDADTKQSSLHITASQLSDSASYICVV |
| | domain (fully | NSAQGLYIPTFGRGTSLIVHP |
| | glycosylated form) | |
| 8 | MAGEA4 specific TCR | LDHEN |
| | beta CDR1 | |
| 9 | MAGEA4 specific TCR | SRFATG |
| | beta CDR2 | |
| 10 | MAGEA4 specific TCR | ASSSDQNSGDPYEQYF |
| | beta CDR3 | |
| 11 | MAGEA4 specific TCR | DVKVTQSSRYLVKRTGEKVFLECVQDLDHENMFWYRQDPGLGLR |
| | beta chain variable | LIYFSRFATGKEKGDIPEGYSVSREKKERFSLILESASTQQTSMYLC |
| | domain | ASSSDQNSGDPYEQYFGPGTRLTVT |
| | (monoglycosylated/ | |
| | aglycosylated form) | |
| 12 | MAGEA4 specific beta | DVKVTQSSRYLVKRTGEKVFLECVQDLDHENMFWYRQDPGLGLR |
| | chain variable domain | LIYFSRFATGKEKGDIPEGYSVSREKKERFSLILESASTNQTSMYLC |
| | (fully glycosylated form) | ASSSDQNSGDPYEQYFGPGTRLTVT |
| 13 | MAGEA4 specific TCR | ANQVEQSPQSLIILEGKNVTLQCQYT |
| | alpha FR1 | |
| | (monoglycosylated | |
| | form) | |
| 14 | MAGEA4 specific TCR | ANQVEQSPQSLIILEGKQVTLQCQYT |
| | alpha FR1 | |
| | (aglycosylated form) | |
| 15 | MAGEA4 specific TCR | ANQVEQSPQSLIILEGKNVTLQCNYT |
| | alpha FR1 (fully | |
| | glycosylated form) | |
| 16 | MAGEA4 specific TCR | LRWYKQDTGRGPVSLTI |
| | alpha FR2 | |
| | (monoglycosylated/ | |
| | aglycosylated form) | |
| 17 | MAGEA4 specific TCR | KSNGRYTATLDADTKQSSLHITASQLSDSASYIC |
| | alpha FR3 | |
| | (monoglycosylated/ | |
| | aglycosylated form) | |
| 18 | MAGEA4 specific TCR | GRGTSLIVHP |
| | alpha FR4 | |
| | (monoglycosylated/ | |
| | aglycosylated form) | |
| 19 | MAGEA4 specific TCR | DVKVTQSSRYLVKRTGEKVFLECVQD |
| | beta FR1 | |
| | (monoglycosylated/ | |
| | aglycosylated form) | |
| 20 | MAGEA4 specific TCR | MFWYRQDPGLGLRLIYF |
| | beta FR2 | |
| | (monoglycosylated/ | |
| | aglycosylated form) | |
| 21 | MAGEA4 specific TCR | KEKGDIPEGYSVSREKKERFSLILESASTQQTSMYLC |
| | beta FR3 | |
| | (monoglycosylated/ | |
| | aglycosylated form) | |
| 22 | MAGEA4 specific TCR | KEKGDIPEGYSVSREKKERFSLILESASTNQTSMYLC |
| | beta FR3 (fully | |
| | glycosylated form) | |
| 23 | MAGEA4 specific TCR | GPGTRLTVT |
| | beta FR4 | |
| | (monoglycosylated/ | |
| | aglycosylated form) | |
| 24 | MAGEA4 specific TCR | ANQVEQSPQSLIILEGKNVTLQCQYTVSPFSNLRWYKQDTGRGPV |
| | alpha chain | SLTILTFSENTKSNGRYTATLDADTKQSSLHITASQLSDSASYICVV |
| | (monoglycosylated | NSAQGLYIPTFGRGTSLIVHPYIQKPDPAVYQLRDSKSSDKSVCLFT |
| | form) | DFDSQTQVSQSKDSDVYITDKCVLDMRSMDFKSNSAVAWSQKSD |
| | | FACANAFQNSIIPEDT |
| 25 | MAGEA4 specific TCR | ANQVEQSPQSLIILEGKQVTLQCQYTVSPFSNLRWYKQDTGRGPV |
| | alpha chain | SLTILTFSENTKSNGRYTATLDADTKQSSLHITASQLSDSASYICVV |
| | (aglycosylated form) | NSAQGLYIPTFGRGTSLIVHPYIQKPDPAVYQLRDSKSSDKSVCLFT |
| | | DFDSQTQVSQSKDSDVYITDKCVLDMRSMDFKSNSAVAWSQKSD |
| | | FACANAFQNSIIPEDT |
| 26 | MAGEA4 specific TCR | ANQVEQSPQSLIILEGKNVTLQCNYTVSPFSNLRWYKQDTGRGPV |
| | alpha chain (fully | SLTILTFSENTKSNGRYTATLDADTKQSSLHITASQLSDSASYICVV |
| | glycosylated form) | NSAQGLYIPTFGRGTSLIVHPYIQKPDPAVYQLRDSKSSDKSVCLFT |
| | | DFDSQTNVSQSKDSDVYITDKCVLDMRSMDFKSNSAVAWSNKSD |
| | | FACANAFNNSIIPEDT |
| 27 | MAGEA4 specific TCR | DVKVTQSSRYLVKRTGEKVFLECVQDLDHENMFWYRQDPGLGLR |
| | beta chain | LIYFSRFATGKEKGDIPEGYSVSREKKERFSLILESASTQQTSMYLC |
| | (monoglycosylated/ | ASSSDQNSGDPYEQYFGPGTRLTVTEDLKNVFPPEVAVFEPSEAEI |
| | aglycosylated form) | SHTQKATLVCLATGFYPDHVELSWWVNGKEVHSGVCTDPQPLKE |
| | | QPALQDSRYALSSRLRVSATFWQDPRNHFRCQVQFYGLSENDEW |
| | | TQDRAKPVTQIVSAEAWGRAD |
| 28 | MAGEA4 specific TCR | DVKVTQSSRYLVKRTGEKVFLECVQDLDHENMFWYRQDPGLGLR |
| | beta chain (fully | LIYFSRFATGKEKGDIPEGYSVSREKKERFSLILESASTNQTSMYLC |
| | glycosylated form) | ASSSDQNSGDPYEQYFGPGTRLTVTEDLKNVFPPEVAVFEPSEAEI |
| | | SHTQKATLVCLATGFYPDHVELSWWVNGKEVHSGVCTDPQPLKE |
| | | QPALNDSRYALSSRLRVSATFWQDPRNHFRCQVQFYGLSENDEW |
| | | TQDRAKPVTQIVSAEAWGRAD |
| 29 | MAGEA4 specific TCR | YIQKPDPAVYQLRDSKSSDKSVCLFTDFDSQTQVSQSKDSDVYITD |
| | alpha chain constant | KCVLDMRSMDFKSNSAVAWSQKSDFACANAFQNSIIPEDT |
| | domain | |
| | (monoglycosylated/ | |
| | aglycosylated form) | |
| 30 | MAGEA4 specific TCR | YIQKPDPAVYQLRDSKSSDKSVCLFTDFDSQTNVSQSKDSDVYITD |
| | alpha chain constant | KCVLDMRSMDFKSNSAVAWSNKSDFACANAFNNSIIPEDT |
| | domain (fully | |
| | glycosylated form) | |
| 31 | MAGEA4 specific TCR | EDLKNVFPPEVAVFEPSEAEISHTQKATLVCLATGFYPDHVELSWW |
| | beta chain constant | VNGKEVHSGVCTDPQPLKEQPALQDSRYALSSRLRVSATFWQDP |
| | domain | RNHFRCQVQFYGLSENDEWTQDRAKPVTQIVSAEAWGRAD |
| | (monoglycosylated/ | |
| | aglycosylated form) | |
| 32 | MAGEA4 specific TCR | EDLKNVFPPEVAVFEPSEAEISHTQKATLVCLATGFYPDHVELSWW |
| | beta chain constant | VNGKEVHSGVCTDPQPLKEQPALNDSRYALSSRLRVSATFWQDP |
| | domain fully | RNHFRCQVQFYGLSENDEWTQDRAKPVTQIVSAEAWGRAD |
| | glycosylated form | |
| 33 | Truncated IgG Hinge | DKTHTCPPCP |
| 34 | Linker | GGGGS |
| 35 | Linker | GGGSG |
| 36 | Linker | GGSGG |
| 37 | Linker | GSGGG |
| 38 | Linker | GSGGGP |
| 39 | Linker | GGEPS |
| 40 | Linker | GGEGGGP |
| 41 | Linker | GGEGGGSEGGGS |
| 42 | Linker | GGGSGGGG |
| 43 | Linker | GGGGSGGGGSGGGGSGGGGS |
| 44 | Linker | EAAAK |
| 45 | Linker | EAAAKEAAAKEAAAK |
| 46 | Linker | GGGGSGGGGSGGGGSGGGGSGGGS |
| 47 | Hinge | EPKSSDKTHTCPPCP |
| 48 | Hinge | ESKYGPPCPSCP |
| 49 | Fc region with knob | APELLGGPSVFLFPPKPKDTLMISRTPEVTCVVVDVSHEDPEVKFN |
| | mutation | WYVDGVEVHNAKTKPREEQYGSTYRVVSVLTVLHQDWLNGKEYK |
| | | CKVSNKALPAPIEKTISKAKGQPREPQVYTLPPSRDELTKNQVSLW |
| | | CLVKGFYPSDIAVEWESNGQPENNYKTTPPVLDSDGSFFLYSKLTV |
| | | DKSRWQQGNVFSCSVMHEALHNHYTQKSLSLSPGK |
| 50 | Fc region with hole | APELLGGPSVFLFPPKPKDTLMISRTPEVTCVVVDVSHEDPEVKFN |
| | mutations | WYVDGVEVHNAKTKPREEQYGSTYRVVSVLTVLHQDWLNGKEYK |
| | | CKVSNKALPAPIEKTISKAKGQPREPQVYTLPPSRDELTKNQVSLS |
| | | CAVKGFYPSDIAVEWESNGQPENNYKTTPPVLDSDGSFFLVSKLT |
| | | VDKSRWQQGNVFSCSVMHEALHNHYTQKSLSLSPGK |
| 51 | Human IgG1 Fc region | APELLGGPSVFLFPPKPKDTLMISRTPEVTCVVVDVSHEDPEVKFN |
| | (CH2 and CH3 | WYVDGVEVHNAKTKPREEQYNSTYRVVSVLTVLHQDWLNGKEYK |
| | domains), unmodified | CKVSNKALPAPIEKTISKAKGQPREPQVYTLPPSRDELTKNQVSLT |
| | | CLVKGFYPSDIAVEWESNGQPENNYKTTPPVLDSDGSFFLYSKLTV |
| | | DKSRWQQGNVFSCSVMHEALHNHYTQKSLSLSPGK |
| 52 | Exemplary third chain | DKTHTCPPCPAPELLGGPSVFLFPPKPKDTLMISRTPEVTCVVVDV |
| | (amino acid) comprising | SHEDPEVKFNWYVDGVEVHNAKTKPREEQYGSTYRVVSVLTVLH |
| | an hIgG1 Fc chain (with | QDWLNGKEYKCKVSNKALPAPIEKTISKAKGQPREPQVYTLPPSR |
| | truncated hinge; N297G, | DELTKNQVSLWCLVKGFYPSDIAVEWESNGQPENNYKTTPPVLDS |
| | T366W substitutions) | DGSFFLYSKLTVDKSRWQQGNVFSCSVMHEALHNHYTQKSLSLS |
| | | PGK |
| 53 | Exemplary third chain | GACAAGACCCACACCTGTCCTCCATGTCCTGCTCCAGAACTGCT |
| | (DNA) comprising a | CGGCGGACCTTCCGTGTTCCTGTTTCCTCCAAAGCCTAAGGAC |
| | hIgG1 Fc chain (with | ACCCTGATGATCTCTCGGACCCCTGAAGTGACCTGCGTGGTGG |
| | truncated hinge; | TGGATGTGTCTCACGAGGATCCCGAAGTGAAGTTCAATTGGTAC |
| | N297G, T366W | GTGGACGGCGTGGAAGTGCACAACGCCAAGACCAAGCCTAGA |
| | substitutions) | GAGGAACAGTACGGCTCCACCTACAGAGTGGTGTCCGTGCTGA |
| | | CAGTGCTGCACCAGGATTGGCTGAACGGCAAAGAGTACAAGTG |
| | | CAAGGTGTCCAACAAGGCCCTGCCTGCCCCAATCGAAAAGACC |
| | | ATCTCCAAGGCCAAGGGCCAGCCTAGGGAACCCCAGGTTTACA |
| | | CCCTGCCTCCAAGCCGGGATGAGCTGACCAAGAACCAGGTGTC |
| | | CCTGTGGTGCCTGGTCAAGGGCTTCTACCCTTCCGATATCGCC |
| | | GTGGAATGGGAGAGCAATGGCCAGCCTGAGAACAACTACAAGA |
| | | CAACCCCTCCTGTGCTGGACTCCGACGGCTCATTCTTCCTGTAC |
| | | TCCAAGCTGACCGTGGACAAGTCCAGATGGCAGCAGGGCAACG |
| | | TGTTCTCCTGCTCCGTGATGCACGAGGCCCTGCACAATCACTAC |
| | | ACCCAGAAGTCCCTGTCTCTGTCCCCTGGCAAA |
| 54 | Exemplary second | ANQVEQSPQSLIILEGKQVTLQCQYTVSPFSNLRWYKQDTGRGPV |
| | chain (amino acid) | SLTILTFSENTKSNGRYTATLDADTKQSSLHITASQLSDSASYICVV |
| | comprising a TCRα | NSAQGLYIPTFGRGTSLIVHPYIQKPDPAVYQLRDSKSSDKSVCLFT |
| | variable domain and | DFDSQTQVSQSKDSDVYITDKCVLDMRSMDFKSNSAVAWSQKSD |
| | constant domain | FACANAFQNSIIPEDTDKTHTCPPCPAPELLGGPSVFLFPPKPKDTL |
| | (aglycosylated variant) | MISRTPEVTCVVVDVSHEDPEVKFNWYVDGVEVHNAKTKPREEQY |
| | linked to an hIgG1 Fc | GSTYRVVSVLTVLHQDWLNGKEYKCKVSNKALPAPIEKTISKAKGQ |
| | chain (with truncated | PREPQVYTLPPSRDELTKNQVSLSCAVKGFYPSDIAVEWESNGQP |
| | hinge, N297G T366S, | ENNYKTTPPVLDSDGSFFLVSKLTVDKSRWQQGNVFSCSVMHEAL |
| | L368A, and Y407V | HNHYTQKSLSLSPGK |
| | substitutions) | |
| 55 | Exemplary second | CCAACCAGGTGGAACAGTCCCCTCAGAGCCTGATCATCCTGGA |
| | chain (DNA) comprising | AGGCAAGCAGGTGACCCTGCAGTGCCAGTACACCGTGTCTCCC |
| | aTCRα variable domain | TTCTCCAACCTGCGGTGGTACAAGCAGGATACCGGCAGAGGAC |
| | and constant domain | CTGTGTCTCTGACCATCCTGACCTTCTCCGAGAACACCAAGTCC |
| | (aglycosylated) linked | AACGGCCGGTACACCGCCACACTGGATGCTGACACAAAGCAGT |
| | to anhIgG1 Fc chain | CCAGCCTGCACATCACCGCCTCTCAGCTGTCTGACTCCGCCTC |
| | (with truncated hinge, | CTACATCTGCGTGGTCAATTCTGCCCAGGGCCTGTACATCCCCA |
| | N297G T366S, L368A, | CCTTCGGAAGAGGCACCAGCCTGATCGTGCACCCCTACATCCA |
| | Y407V substitutions) | GAAACCTGATCCTGCCGTGTACCAGCTGAGAGACAGCAAGTCC |
| | | AGCGACAAGAGCGTGTGCCTGTTCACCGACTTCGACAGCCAGA |
| | | CCCAAGTGTCCCAGAGCAAGGACAGCGACGTGTACATCACCGA |
| | | TAAGTGCGTGCTGGACATGCGGAGCATGGACTTCAAGAGCAAC |
| | | AGCGCCGTGGCCTGGTCCCAAAAGAGCGATTTCGCCTGCGCCA |
| | | ACGCCTTCCAAAACAGCATTATCCCCGAGGACACAGACAAGAC |
| | | CCACACCTGTCCTCCATGTCCTGCTCCAGAACTGCTCGGCGGA |
| | | CCTTCCGTGTTCCTGTTTCCTCCAAAGCCTAAGGACACCCTGAT |
| | | GATCTCTCGGACCCCTGAAGTGACCTGCGTGGTGGTGGATGTG |
| | | TCTCACGAGGATCCCGAAGTGAAGTTCAATTGGTACGTGGACG |
| | | GCGTGGAAGTGCACAACGCCAAGACCAAGCCTAGAGAGGAACA |
| | | GTACGGCTCCACCTACAGAGTGGTGTCCGTGCTGACAGTGCTG |
| | | CACCAGGATTGGCTGAACGGCAAAGAGTACAAGTGCAAGGTGT |
| | | CCAACAAGGCCCTGCCTGCCCCAATCGAAAAGACCATCTCCAA |
| | | GGCCAAGGGCCAGCCTAGGGAACCCCAGGTTTACACCCTGCCT |
| | | CCAAGCCGGGATGAGCTGACCAAGAACCAGGTGTCCCTGTCCT |
| | | GCGCCGTCAAGGGCTTCTACCCTTCCGATATCGCCGTGGAATG |
| | | GGAGAGCAATGGCCAGCCTGAGAACAACTACAAGACAACCCCT |
| | | CCTGTGCTGGACTCCGACGGCTCATTCTTCCTGGTGTCCAAGC |
| | | TGACCGTGGACAAGTCCAGATGGCAGCAGGGCAACGTGTTCTC |
| | | CTGCTCCGTGATGCACGAGGCCCTGCACAATCACTACACCCAG |
| | | AAGTCCCTGTCTCTGTCCCCTGGCAAA |
| 56 | Exemplary first chain | AIQMTQSPSSLSASVGDRVTITCRASQDIRNYLNWYQQKPGKAPK |
| | (amino acid) comprising | LLIYYTSRLESGVPSRFSGSGSGTDYTLTISSLQPEDFATYYCQQG |
| | a UCHT1(U28) Anti- | NTLPWTFGQGTKVEIKGGGGSGGGGSGGGGSGGGGSGGGSEV |
| | CD3 scFv linked to | QLVESGGGLVQPGGSLRLSCAASGYSFTGYAMNWVRQAPGKGL |
| | aTCRβ variable domain | EWVALINPYKGVSTYNQKFKDRFTFSVDKSKNTAYLQMNSLRAED |
| | and constant domain | TAVYYCARSGYYGDSDWYFDVWGQGTLVTVSSGGGGSDVKVTQ |
| | (aglycosylated) | SSRYLVKRTGEKVFLECVQDLDHENMFWYRQDPGLGLRLIYFSRF |
| | | ATGKEKGDIPEGYSVSREKKERFSLILESASTQQTSMYLCASSSDQ |
| | | NSGDPYEQYFGPGTRLTVTEDLKNVFPPEVAVFEPSEAEISHTQKA |
| | | TLVCLATGFYPDHVELSWWVNGKEVHSGVCTDPQPLKEQPALQD |
| | | SRYALSSRLRVSATFWQDPRNHFRCQVQFYGLSENDEWTQDRAK |
| | | PVTQIVSAEAWGRAD |
| 57 | Exemplary first chain | GCAATTCAGATGACTCAAAGTCCGAGTTCTTTGTCTGCGAGCGT |
| | (DNA) comprising a | TGGGGATCGTGTGACGATTACATGTCGCGCCAGCCAAGACATT |
| | UCHT1(U28) Anti-CD3 | CGCAATTATCTGAACTGGTATCAACAGAAGCCAGGGAAGGCGC |
| | scFv linked to aTCRβ | CGAAACTGTTAATCTATTACACATCCCGCCTGGAATCGGGAGTT |
| | variable domain and | CCTTCGCGCTTTAGCGGCTCCGGCAGCGGCACTGATTACACGT |
| | constant domain | TGACCATTAGCAGCTTACAGCCTGAAGATTTTGCCACTTATTATT |
| | (monoglycoslylated/ | GTCAGCAGGGAAACACTCTGCCATGGACCTTCGGGCAGGGCAC |
| | aglycosylated) | GAAAGTGGAAATCAAGGGTGGTGGTGGCAGTGGTGGAGGCGG |
| | | ATCAGGGGGCGGGGGAAGCGGCGGAGGCGGTTCAGGTGGCG |
| | | GAAGCGAGGTGCAGTTAGTGGAGAGTGGCGGCGGTTTGGTTCA |
| | | ACCAGGTGGTTCCTTGCGCCTGTCATGCGCGGCTTCAGGCTAC |
| | | TCATTCACCGGCTATGCCATGAATTGGGTGCGTCAGGCACCCG |
| | | GTAAAGGTTTAGAATGGGTGGCGCTGATTAATCCGTATAAGGG |
| | | GGTATCTACCTACAACCAGAAGTTCAAAGATCGCTTCACATTTTC |
| | | TGTAGACAAATCTAAAAATACTGCCTACTTGCAGATGAATAGTCT |
| | | GCGTGCAGAAGACACAGCAGTTTACTACTGCGCCCGCTCGGGT |
| | | TACTACGGCGATTCAGACTGGTACTTTGATGTGTGGGGACAGG |
| | | GAACGTTAGTAACAGTGTCCTCCGGTGGTGGCGGCTCCGACGT |
| | | GAAAGTGACCCAGAGCAGCAGATACCTGGTCAAGAGAACCGGC |
| | | GAGAAGGTGTTCCTGGAATGCGTGCAGGACCTGGACCACGAGA |
| | | ACATGTTTTGGTACAGACAGGACCCCGGCCTGGGCCTGAGACT |
| | | GATCTATTTCTCCAGATTCGCCACCGGCAAAGAGAAGGGCGAT |
| | | ATCCCCGAGGGCTACTCCGTGTCCAGAGAGAAGAAAGAGCGGT |
| | | TCTCCCTGATCCTGGAAAGCGCCTCTACCCAGCAGACCTCTATG |
| | | TACCTGTGCGCCTCCTCCAGCGACCAGAACTCTGGCGATCCTT |
| | | ACGAGCAGTACTTCGGCCCAGGCACCAGACTGACCGTGACAGA |
| | | AGATCTGAAGAACGTGTTCCCACCTGAGGTGGCCGTGTTCGAG |
| | | CCTTCTGAGGCCGAGATCAGCCACACCCAGAAAGCCACCCTCG |
| | | TGTGTCTGGCCACAGGTTTCTACCCCGACCACGTGGAACTGTC |
| | | TTGGTGGGTCAACGGCAAAGAGGTGCACTCCGGCGTGTGCACC |
| | | GATCCCCAGCCCCTGAAAGAACAGCCCGCCCTGCAAGACAGCA |
| | | GATACGCCCTGAGCAGCAGGCTGAGAGTGTCCGCCACCTTCTG |
| | | GCAGGACCCCCGGAATCACTTCAGGTGCCAGGTGCAGTTCTAC |
| | | GGCCTGAGCGAGAACGACGAGTGGACCCAGGACAGAGCCAAG |
| | | CCCGTGACCCAGATCGTGTCTGCCGAAGCCTGGGGCAGAGCT |
| | | GAT |
| 58 | Alternative exemplary | ANQVEQSPQSLIILEGKNVTLQCQYTVSPFSNLRWYKQDTGRGPV |
| | second chain (amino | SLTILTFSENTKSNGRYTATLDADTKQSSLHITASQLSDSASYICVV |
| | acid) comprising a | NSAQGLYIPTFGRGTSLIVHPYIQKPDPAVYQLRDSKSSDKSVCLFT |
| | TCRα variable domain | DFDSQTQVSQSKDSDVYITDKCVLDMRSMDFKSNSAVAWSQKSD |
| | and constant domain | FACANAFQNSIIPEDTDKTHTCPPCPAPELLGGPSVFLFPPKPKDTL |
| | (mono-glycosylated) | MISRTPEVTCVVVDVSHEDPEVKFNWYVDGVEVHNAKTKPREEQY |
| | linked to an hIgG1 Fc | GSTYRVVSVLTVLHQDWLNGKEYKCKVSNKALPAPIEKTISKAKGQ |
| | chain (with truncated | PREPQVYTLPPSRDELTKNQVSLSCAVKGFYPSDIAVEWESNGQP |
| | hinge, N297G, T366S, | ENNYKTTPPVLDSDGSFFLVSKLTVDKSRWQQGNVFSCSVMHEAL |
| | L368A, Y407V | HNHYTQKSLSLSPGK |
| | substitutions) | |
| 59 | Alternative exemplary | GCCAACCAGGTGGAACAGTCCCCTCAGAGCCTGATCATCCTGG |
| | second chain (DNA) | AAGGCAAGAACGTGACCCTGCAGTGCCAGTACACCGTGTCTCC |
| | comprising a TCRα | CTTCTCCAACCTGCGGTGGTACAAGCAGGATACCGGCAGAGGA |
| | variable domain and | CCTGTGTCTCTGACCATCCTGACCTTCTCCGAGAACACCAAGTC |
| | constant domain | CAACGGCCGGTACACCGCCACACTGGATGCTGACACAAAGCAG |
| | (mono-glycosylated) | TCCAGCCTGCACATCACCGCCTCTCAGCTGTCTGACTCCGCCT |
| | linked to an hIgG1 Fc | CCTACATCTGCGTGGTCAATTCTGCCCAGGGCCTGTACATCCC |
| | chain (with truncated | CACCTTCGGAAGAGGCACCAGCCTGATCGTGCACCCCTACATC |
| | hinge, N297G, T366S, | CAGAAACCTGATCCTGCCGTGTACCAGCTGAGAGACAGCAAGT |
| | L368A, Y407V | CCAGCGACAAGAGCGTGTGCCTGTTCACCGACTTCGACAGCCA |
| | substitutions) | GACCCAAGTGTCCCAGAGCAAGGACAGCGACGTGTACATCACC |
| | | GATAAGTGCGTGCTGGACATGCGGAGCATGGACTTCAAGAGCA |
| | | ACAGCGCCGTGGCCTGGTCCCAAAAGAGCGATTTCGCCTGCGC |
| | | CAACGCCTTCCAAAACAGCATTATCCCCGAGGACACAGACAAG |
| | | ACCCACACCTGTCCTCCATGTCCTGCTCCAGAACTGCTCGGCG |
| | | GACCTTCCGTGTTCCTGTTTCCTCCAAAGCCTAAGGACACCCTG |
| | | ATGATCTCTCGGACCCCTGAAGTGACCTGCGTGGTGGTGGATG |
| | | TGTCTCACGAGGATCCCGAAGTGAAGTTCAATTGGTACGTGGA |
| | | CGGCGTGGAAGTGCACAACGCCAAGACCAAGCCTAGAGAGGA |
| | | ACAGTACGGCTCCACCTACAGAGTGGTGTCCGTGCTGACAGTG |
| | | CTGCACCAGGATTGGCTGAACGGCAAAGAGTACAAGTGCAAGG |
| | | TGTCCAACAAGGCCCTGCCTGCCCCAATCGAAAAGACCATCTC |
| | | CAAGGCCAAGGGCCAGCCTAGGGAACCCCAGGTTTACACCCTG |
| | | CCTCCAAGCCGGGATGAGCTGACCAAGAACCAGGTGTCCCTGT |
| | | CCTGCGCCGTCAAGGGCTTCTACCCTTCCGATATCGCCGTGGA |
| | | ATGGGAGAGCAATGGCCAGCCTGAGAACAACTACAAGACAACC |
| | | CCTCCTGTGCTGGACTCCGACGGCTCATTCTTCCTGGTGTCCAA |
| | | GCTGACCGTGGACAAGTCCAGATGGCAGCAGGGCAACGTGTTC |
| | | TCCTGCTCCGTGATGCACGAGGCCCTGCACAATCACTACACCC |
| | | AGAAGTCCCTGTCTCTGTCCCCTGGCAAA |
| 60 | Alternative exemplary | ANQVEQSPQSLIILEGKNVTLQCNYTVSPFSNLRWYKQDTGRGPV |
| | second chain (amino | SLTILTFSENTKSNGRYTATLDADTKQSSLHITASQLSDSASYICVV |
| | acid) comprising a | NSAQGLYIPTFGRGTSLIVHPYIQKPDPAVYQLRDSKSSDKSVCLFT |
| | TCRα variable domain | DFDSQTNVSQSKDSDVYITDKCVLDMRSMDFKSNSAVAWSNKSD |
| | and constant domain | FACANAFNNSIIPEDTDKTHTCPPCPAPELLGGPSVFLFPPKPKDTL |
| | (fully glycosylated) | MISRTPEVTCVVVDVSHEDPEVKFNWYVDGVEVHNAKTKPREEQY |
| | linked to an hIgG1 Fc | GSTYRVVSVLTVLHQDWLNGKEYKCKVSNKALPAPIEKTISKAKGQ |
| | chain (with truncated | PREPQVYTLPPSRDELTKNQVSLSCAVKGFYPSDIAVEWESNGQP |
| | hinge, N297G, T366S, | ENNYKTTPPVLDSDGSFFLVSKLTVDKSRWQQGNVFSCSVMHEAL |
| | L368A, Y407V | HNHYTQKSLSLSPGK |
| | substitutions) | |
| 61 | Alternative exemplary | AIQMTQSPSSLSASVGDRVTITCRASQDIRNYLNWYQQKPGKAPK |
| | first chain (amino acid) | LLIYYTSRLESGVPSRFSGSGSGTDYTLTISSLQPEDFATYYCQQG |
| | comprising a | NTLPWTFGQGTKVEIKGGGGSGGGGSGGGGSGGGGSGGGSEV |
| | UCHT1(U28) Anti-CD3 | QLVESGGGLVQPGGSLRLSCAASGYSFTGYAMNWVRQAPGKGL |
| | scFv linked to a TCRβ | EWVALINPYKGVSTYNQKFKDRFTFSVDKSKNTAYLQMNSLRAED |
| | variable domain and | TAVYYCARSGYYGDSDWYFDVWGQGTLVTVSSGGGGSDVKVTQ |
| | constant domain (fully | SSRYLVKRTGEKVFLECVQDLDHENMFWYRQDPGLGLRLIYFSRF |
| | glycosylated) | ATGKEKGDIPEGYSVSREKKERFSLILESASTNQTSMYLCASSSDQ |
| | | NSGDPYEQYFGPGTRLTVTEDLKNVFPPEVAVFEPSEAEISHTQKA |
| | | TLVCLATGFYPDHVELSWWVNGKEVHSGVCTDPQPLKEQPALND |
| | | SRYALSSRLRVSATFWQDPRNHFRCQVQFYGLSENDEWTQDRAK |
| | | PVTQIVSAEAWGRAD |
| 62 | Anti-CD3 scFv UCHT1 | AIQMTQSPSSLSASVGDRVTITCRASQDIRNYLNWYQQKPGKAPK |
| | (U0) | LLIYYTSRLESGVPSRFSGSGSGTDYTLTISSLQPEDFATYYCQQG |
| | | NTLPWTFGQGTKVEIKGGGGSGGGGSGGGGSGGGGSGGGSEV |
| | | QLVESGGGLVQPGGSLRLSCAASGYSFTGYTMNWVRQAPGKGLE |
| | | WVALINPYKGVSTYNQKFKDRFTISVDKSKNTAYLQMNSLRAEDTA |
| | | VYYCARSGYYGDSDWYFDVWGQGTLVTVSS |
| 63 | Anti-CD3 scFv U28 | AIQMTQSPSSLSASVGDRVTITCRASQDIRNYLNWYQQKPGKAPK |
| | | LLIYYTSRLESGVPSRFSGSGSGTDYTLTISSLQPEDFATYYCQQG |
| | | NTLPWTFGQGTKVEIKGGGGSGGGGSGGGGSGGGGSGGGSEV |
| | | QLVESGGGLVQPGGSLRLSCAASGYSFTGYAMNWVRQAPGKGL |
| | | EWVALINPYKGVSTYNQKFKDRFTFSVDKSKNTAYLQMNSLRAED |
| | | TAVYYCARSGYYGDSDWYFDVWGQGTLVTVSS |
| 64 | Anti-MAGE-A4 TCR | ANQVEQSPQSLIILEGKNVTLQCNYTVSPFSNLRWYKQDTGRGPV |
| | alpha chain constant | SLTILTFSENTKSNGRYTATLDADTKQSSLHITASQLSDSASYICVV |
| | region (fully | NSAQGLYIPTFGRGTSLIVHPYIQKPDPAVYQLRDSKSSDKSVCLFT |
| | glycosylated form) | DFDSQTNVSQSKDSDVYITDKCVLDMRSMDFKSNSAVAWSNKSD |
| | | FACANAFNNSIIPEDT |
| 65 | Anti-MAGE-A4 TCR | EDLKNVFPPEVAVFEPSEAEISHTQKATLVCLATGFYPDHVELSWW |
| | beta chain constant | VNGKEVHSGVCTDPQPLKEQPALNDSRYALSSRLRVSATFWQDP |
| | region (fully | RNHFRCQVQFYGLSENDEWTQDRAKPVTQIVSAEAWGRAD |
| | glycosylated form) | |
| 66 | Anti-CD3 U28 and U0 | AIQMTQSPSSLSASVGDRVTITCRASQDIRNYLNWYQQKPGKAPK |
| | VL | LLIYYTSRLESGVPSRFSGSGSGTDYTLTISSLQPEDFATYYCQQG |
| | | NTLPWTFGQGTKVEIK |
| 67 | Anti-CD3 U28 VH | EVQLVESGGGLVQPGGSLRLSCAASGYSFTGYAMNWVRQAPGK |
| | | GLEWVALINPYKGVSTYNQKFKDRFTFSVDKSKNTAYLQMNSLRA |
| | | EDTAVYYCARSGYYGDSDWYFDVWGQGTLVTVSS |
| 68 | Anti-CD3 scFv U28 | QDIRNY |
| | CDRL1 | |
| 69 | Anti-CD3 scFv U28 | YTS |
| | CDRL2 | |
| 70 | Anti-CD3 scFv U28 | QQGNTLPWT |
| | CDRL3 | |
| 71 | Anti-CD3 scFv U28 | GYSFTGYA |
| | CDRH1 | |
| 72 | Anti-CD3 scFv U28 | INPYKGVS |
| | CDRH2 | |
| 73 | Anti-CD3 scFv U28 | ARSGYYGDSDWYFDV |
| | CDRH3 | |
| 74 | Anti-CD3 scFv U0 VH | EVQLVESGGGLVQPGGSLRLSCAASGYSFTGYTMNWVRQAPGK |
| | | GLEWVALINPYKGVSTYNQKFKDRFTISVDKSKNTAYLQMNSLRAE |
| | | DTAVYYCARSGYYGDSDWYFDVWGQGTLVTVSS |
| 75 | Anti-CD3 scFv U0 | GYSFTGYT |
| | CDRH1 | |
| 76 | Fc region with hole and | APELLGGPSVFLFPPKPKDTLYITREPEVTCVVVDVSHEDPEVKFN |
| | YTE mutation mutations | WYVDGVEVHNAKTKPREEQYGSTYRVVSVLTVLHQDWLNGKEYK |
| | | CKVSNKALPAPIEKTISKAKGQPREPQVYTLPPSRDELTKNQVSLS |
| | | CAVKGFYPSDIAVEWESNGQPENNYKTTPPVLDSDGSFFLVSKLT |
| | | VDKSRWQQGNVFSCSVMHEALHNHYTQKSLSLSPGK |
| 77 | Fc region with knob and | APELLGGPSVFLFPPKPKDTLYITREPEVTCVVVDVSHEDPEVKFN |
| | YTE mutations | WYVDGVEVHNAKTKPREEQYGSTYRVVSVLTVLHQDWLNGKEYK |
| | | CKVSNKALPAPIEKTISKAKGQPREPQVYTLPPSRDELTKNQVSLW |
| | | CLVKGFYPSDIAVEWESNGQPENNYKTTPPVLDSDGSFFLYSKLTV |
| | | DKSRWQQGNVFSCSVMHEALHNHYTQKSLSLSPGK |
| 78 | HLA-A*02 restricted | ALWGPDPAAA, |
| | peptide derived from | |
| | PreProinsulin | |

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the purview of this application and scope of the appended claims.

### Example 1—Multidomain Fc Fusion Molecules with Improved Potency

Multidomain TCR-anti-CD3 fusion protein molecules incorporating a half-life extending Fc domain have been described previously in WO 2020/157211 and shown to be functional. However, it was subsequently found that such molecules have substantially reduced ability to activate T cells in vitro relative to the non Fc-fused format of the molecule and therefore were not considered optimal for therapeutic use. Further engineering was carried out to identify novel molecular formats with improved therapeutic properties.

An initial panel of 8 multidomain Fc fusion molecules with different domain arrangements was constructed, incorporating either an IgG1 or an IgG4 Fc domain. In a first example the TCR variable domains of the multidomain molecules were designed to specifically recognise the HLA-A*02 restricted peptide GVYDGREHTV derived from MAGEA4, with high affinity. Such TCR variable domains are described in WO2017175006. Exemplary sequences of multidomain Fc function molecules according to the invention are also provided in SEQ ID NOs: 52, 54 and 56, SEQ ID NOs: 52, 58 and 56, or SEQ IDs 52, 60 and 61. The immune cell engaging domain was an anti-CD3 scFv fragment. Such sequences can be found in (WO2020157210) and are also provided in SEQ ID NOs: 62 and 63. The ability of these different molecules to drive T cell activation in the presence of antigen positive and antigen negative cells was investigated using an ELISpot assay with IFNγ as a read out of T cell activation.

ELISpot assays were performed using a human IFN-γ ELISPOT kit (BD Biosciences) according to the manufacturer's instructions. Briefly, T2 cells pulsed with 100 nM MAGEA4 peptide were used as antigen positive targets cells; T2 cells pulsed with an irrelevant peptide were used as antigen negative target cells. PBMCs were used as effector cells. The effector target ratio was 0.5:1.

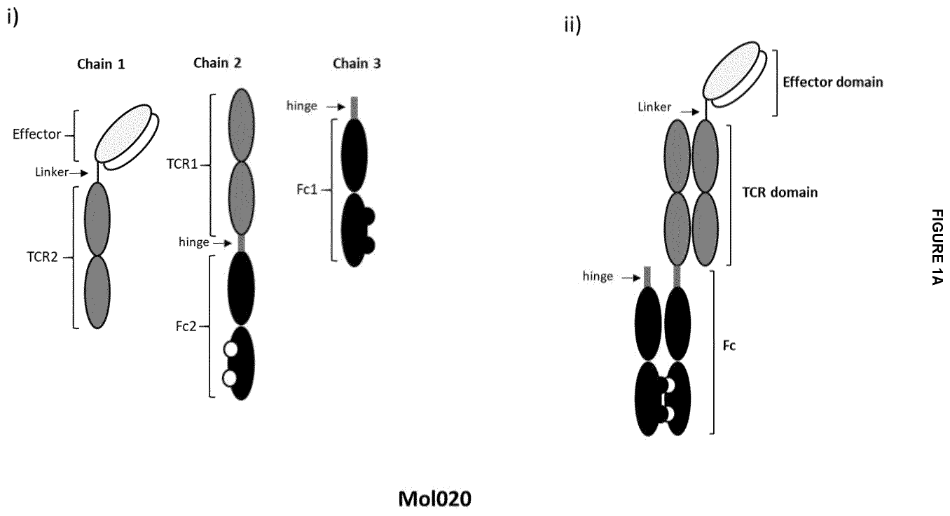

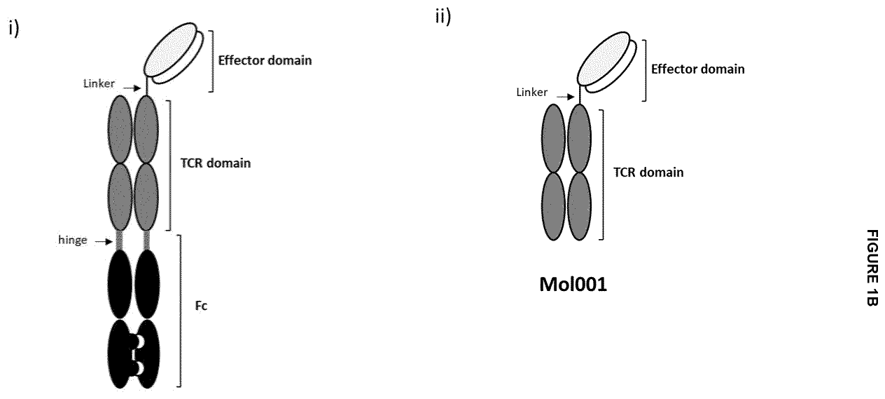

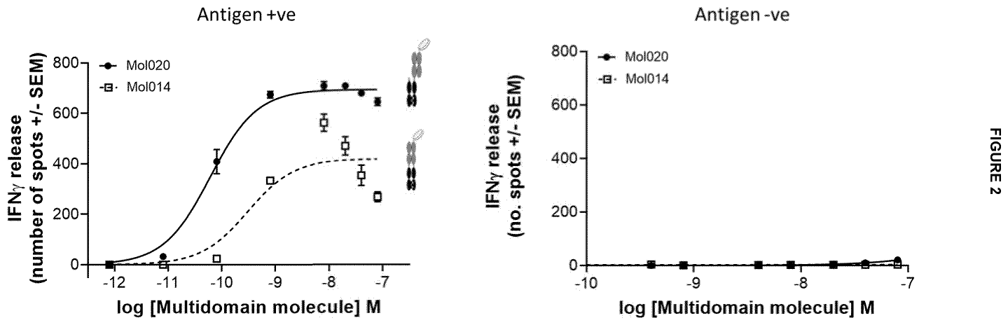

A multidomain molecule having the format shown in FIG. 1A, and termed Mol020, in which each of the functional domains was arranged across three polypeptide chains, was selected based on potency and specificity data.

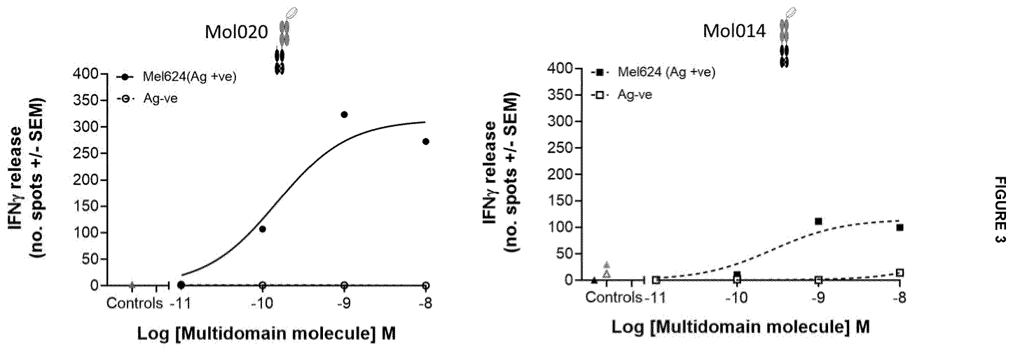

FIG. 2 shows a comparison of T cell activation in the presence and absence of antigen positive cells for two different formats tested (fused to IgG1). The multidomain molecule having the Mol020 format was substantially more potent than the equivalent molecule in an alternative two chain format (Mol014, as shown in FIG. 1B(i)), while retaining a high degree of specificity.

An extended panel of 38 additional Fc fusion formats was subsequently made and screened by ELISpot assay to see if a more potent format could be identified. None of the 38 formats tested showed better activity than Mol020.

To determine if Mol020 would also give rise to a high degree of potency with an alternative TCR, the MAGEA4 specific TCR variable domains in Mol020 and Mol014 were replaced with TCR variable domains that specifically recognise the HLA-A*02 restricted peptide SLLQHLIGL derived from PRAME. Such high affinity PRAME specific TCRs are described in WO2018/234319. IFNγ ELISpot assays were performed as described above except that human melanoma Mel624 cells were used as antigen positive target cells. Mel624 cells in which PRAME was knocked out were used as antigen negative cells.

The data in FIG. 3 demonstrate that Mol020 was again substantially more potent than an alternative two chain format molecule (Mol014), while retaining a high degree of specificity. Overall, these data demonstrate that Mol020 is superior to Mol014 irrespective of the TCR sequence.

### Example 2—Multidomain Fc Fusion Molecules Targeting MAGEA4 Retain a Similar Therapeutic Window to Non-Fc Molecules

The potency and specificity of a multidomain Fc fusion molecule targeting MAGEA4 (Mol020) was compared with an equivalent multidomain molecule without the Fc domain (Mol001, as shown in FIG. 1B(ii)). Mol014 and Mol001 incorporate the same MAGEA4 specific TCR. Such TCR-antiCD3 fusion molecules are further described in WO2017175006.

IFNγ ELISpot assays were carried out as per the manufacturer's instructions. Briefly, Human lung cancer NCI-H1755 cells were used as antigen positive targets cells and human melanoma Mel202 cells were used as antigen negative target cells. Mel202 cells had been prior transduced with HLA-A2 B2M resulting in a very high expression level of HLA-A2; this is known to give rise to an artificially high level of non-specific reactivity, which facilitates comparison. PBMCs were used as effector cells. The effector target ratio was 0.8:1 for Mel202 and 1:1 for NCI-H1755.

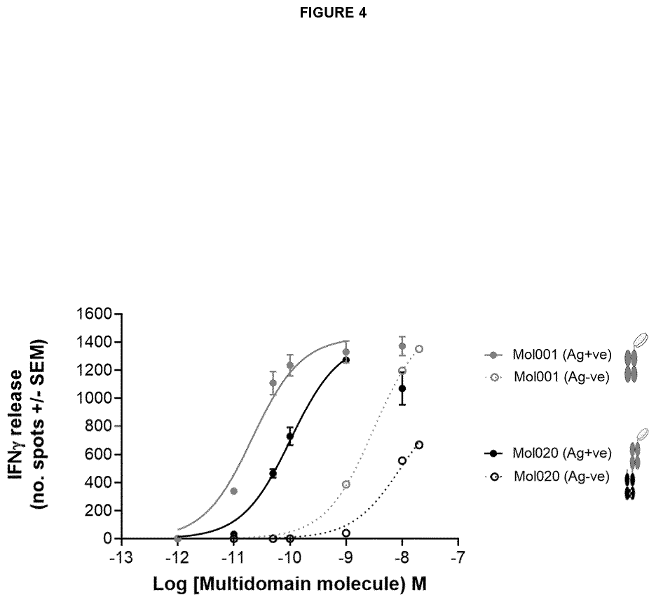

The data presented in FIG. 4 show that while Mol020 gives rise to a less potent response than Mol001, against antigen positive cells, there is also a corresponding reduction in non-specific activity for Mol020 relative to Mol001 at higher concentrations. The difference between on target and off target T cell activation is the same for both molecules. In summary these data show that the therapeutic window for Mol020, relative to the non-Fc fusion Mol001 is maintained.

### Example 3—Multidomain Fc Fusion Molecules Targeting MAGEA4 Demonstrate Extended In Vivo Half-Life Compared to Non-Fc Molecules

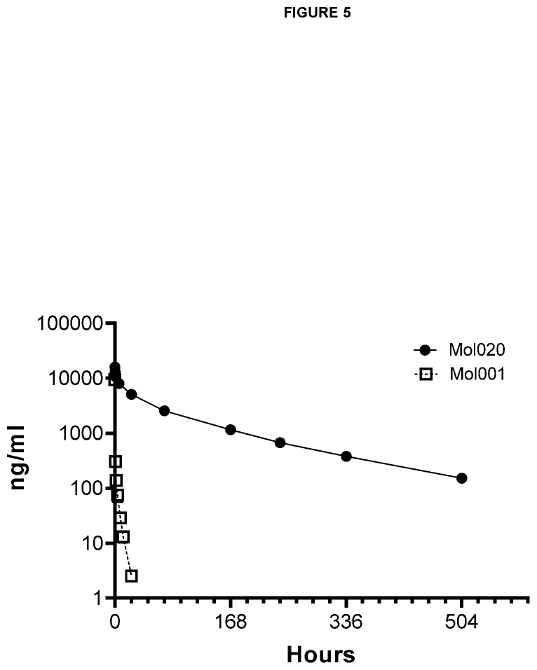

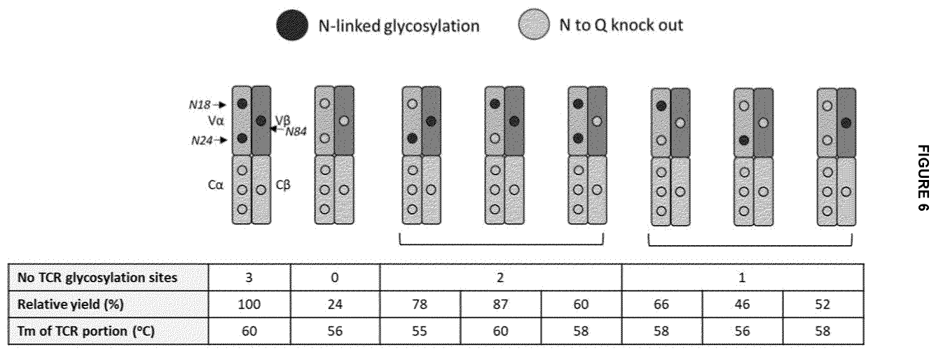

To determine in vivo half-life the pharmacokinetic (PK) properties of Mol020 and Mol001 were assessed in Tg32 SCID mice. Test article was dosed by IV bolus at 1 mg/Kg, 4 mice per compound, with serial sampling of blood over a 21-day period. Sample was detected in serum by electrochemiluminescent immunoassay, with capture on biotinylated MAGEA4 peptide-HLA, and detection with sulfo-tagged anti-scFv antibody. FIG. 5 shows serum concentration overtime. PK parameters were extracted by non-compartmental analysis. Mol001 showed rapid clearance, while Mol020 demonstrated a substantial increase in t_{1/2}, calculated as 4.5d.

### Example 4—Preparation of Multidomain Fc Fusion Molecules that Target MAGEA4

Multidomain Fc fusion molecules (Mol020) that recognise the HLA-A*02 restricted peptide GVYDGREHTV derived from MAGEA4 were prepared using CHO cells and purified via a two-step process.

Briefly, material was generated by transient transfection of expiCHO cells as per the manufacturer's ‘Max-titre’ protocol (Thermo Fisher). Following incubation CHO cultures were centrifuged at 10000 rpm to remove cells and other debris and the supernatant filtered before loading onto a HiTrap Protein L column, with PBS as running buffer. Protein was then eluted with 100 mM Citrate pH 2.5 and the pH raised with 200 ul 2M Tris pH 8.3 per ml eluate prior to concentration in 10000 MWCO concentrators. Further purification was then carried out using a SUP 200 10/300 size exclusion column with PBS as the running buffer. Peak fractions were collected and purity was determined by SDS-PAGE.

### Example 5—Effect of Glycosylation on Yield and Thermal Stability of Multidomain Fc Fusion Molecules that Target MAGEA4

To investigate the effects of glycosylation, sites within the TCR portion of the molecules were identified and mutated. In brief, seven N-linked glycosylation sites were identified in the TCR variable domains and systematically engineered out by replacing N with Q. Molecules were prepared as described in Example 4.

The concentration of purified material was measured by absorbance at 280 nm using a Nanodrop spectrophotometer and extinction coefficient and molecular weight values calculated from the sequence using the Snap Gene software. The final mg of protein was then divided by the overall expression volume in litres.

The UNCLE instrument (Unchained Labs) was used to assess unfolding and thermal aggregation of purified proteins in PBS in parallel quartz capillaries (‘Unis’). The machine uses Static Light Scattering to monitor aggregation and the barycentric mean of tryptophan fluorescence to monitor unfolding of protein samples. Temperature was increased at a rate of 0.5 C/min and protein samples were run at both 50 and 200 μg/ml to monitor denaturation and aggregation. The melting temperature of the TCR portion of the molecule was used as an indicator of thermal stability.

Analysis of glycosylation site variants showed that removal of all seven N-linked glycosylation sites in the TCR portion of the molecule resulted in an approximately 75% reduction in yield. Further investigation demonstrated that removal of the four N-linked glycosylation sites in the TCR constant region did not appear to contribute to the reduction in yield.

Double and single glycosylation site variants in the TCR variable regions were subsequently made and tested. FIG. 6 provides a schematic diagram showing each of the variants, with the locations of the glycosylation sites indicated. The three glycosylation sites in the TCR variable domains are shown at positions N18 and N24 of the alpha chain and N84 of the beta chain. The corresponding relative yield is shown below each of the variants, the melting temperature (Tm) of the TCR portion of the molecule is also shown. The data demonstrate a graduated loss in yield with reduced glycosylation.

### Example 6—Binding Affinity and Kinetics of Mono and Aglycosylated Multidomain Fc Fusion Molecules that Target MAGEA4

In the examples below molecule Mol020v13 is aglycosylated while molecule Mol020v14 contains a single glycosylation site at position N18 in the TCR alpha variable domain. The full amino acid sequence of both molecules is provided in SEQ ID NOs: 52, 54, 56 and 52, 58, 56 respectively.

To verify that the TCR and anti-CD3 portions of the molecule bind to respective target molecules, single cycle kinetics was performed by Surface Plasmon Resonance (SPR) on a T200 BIAcore, followed by a single injection of CD3(γε). Binding to FcRn was also assessed.

Measurement of CD3 and pHLA binding was carried out on an 8K Biacore instrument with streptavidin first immobilised to the chip using EDC-NHS amine coupling followed by binding of biotinylated CD3 or pHLA. Single cycle kinetic experiments were carried out with five injections at either 37° C. (pHLA) or at 25° C. (CD3) in a running buffer of PBS (pH 7.4) with 0.005% surfactant P20. Off and on rates fitted with 1:1 binding kinetic analysis in Biaevaluation software.

Measurement of FcRn binding was carried out using a Biacore T200 instrument. Fc-ImmTAC molecules were immobilised to a CM5 sensor chip by first amine coupling streptavidin to the chip, then binding biotinylated pHLA to the treated chip, then Fc-ImmTAC to this pHLA, taking advantage of the long dissociation time of the TCR-pHLA interaction to effectively immobilise these molecules. Soluble FcRn (Immunitrak) was then injected across the sensor surface at increasing concentrations then given time to dissociate. Response was then plotted against concentration and then fitted with ‘one-site total’ equation in graph pad prism to estimate the K_{D} at equilibrium. These experiments were carried out at 25° C. in 20 mM MES pH 6 with 0.005% surfactant P20 and 137 mM NaCl.

Both Mol020v13 and Mol020v14 showed similarly strong binding to pHLA and CD3, which was also consistent with the non-Fc fused Mol001. In addition, Mol020v13 and Mol020v14 demonstrated binding to FcRn.

TABLE x

| | pHLA | | CD3 | | |
|---|---|---|---|---|---|
| | K_{D} (nM) | T_{1/2} (min) | K_{D} (nM) | T_{1/2} (min) | FcRn |
| Mol001 | 0.21 | 111 | 10 | 3.16 | No |
| Mol020v14 (glyc) | 0.17 | 108 | 16 | 1.9 | Yes |
| Mol020v13 | 0.18 | 89 | 21.7 | 1.32 | Yes |
| (aglyc) | | | | | |

### Example 7 Serum Stability of Mono and Aglycosylated Multidomain Fc Fusion Molecules that Target MAGEA4

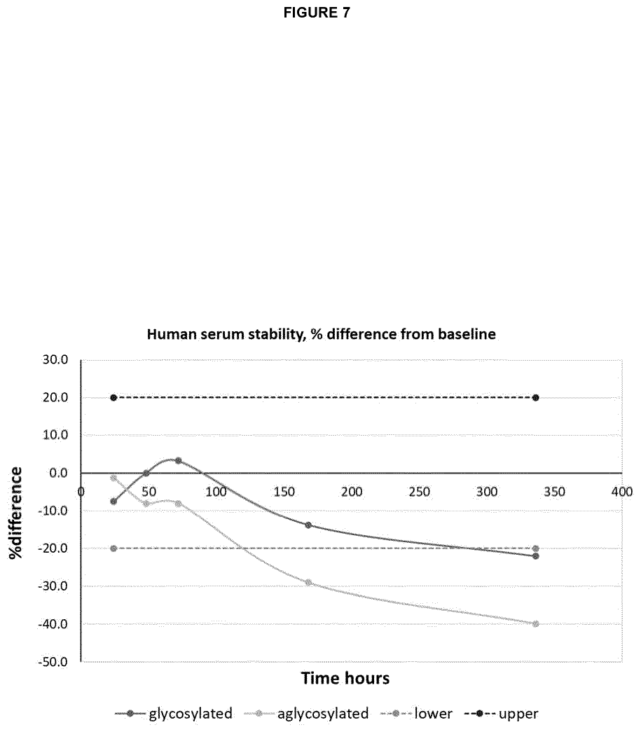

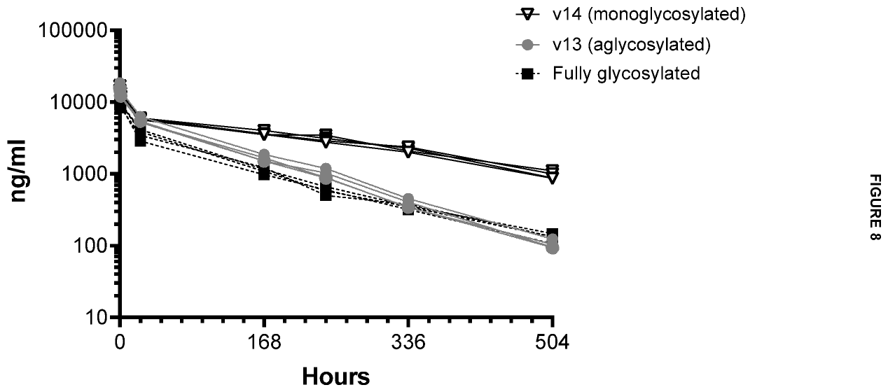

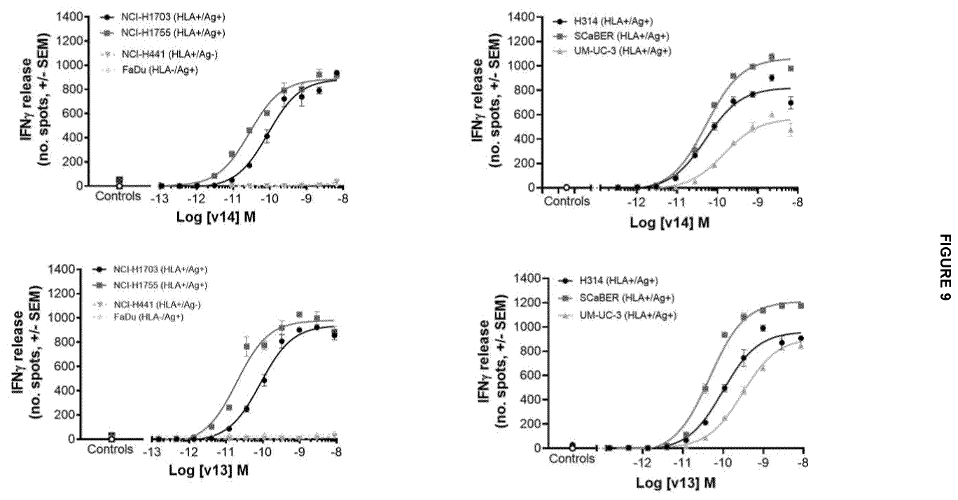

The stability of Mol020v14 and Mol020v13 over time was assessed at 37° C. in human serum in vitro, as a proxy for in vivo stability. Bispecific binding activity was assayed by electrochemiluminescent immunoassay, with capture on biotinylated MAGEA4 peptide-HLA, and detection with sulfo-tagged anti-scFv antibody at various timepoints over a total of 14 days. Percent change from baseline was calculated for each time point and plotted. The results are shown in FIG. 7.

### Example 8—In Vivo Half Life of Mono and Aglycosylated Multidomain Fc Fusion Molecules that Target MAGEA4

A further PK study in Tg32 SCID mice was performed as described in Example 3 to compare the impact of glycosylation on in-vivo half-life. In this example, the monoglycosylated variant mol020v14, is compared with the aglycosylated variant, mol020v13. A fully glycosylated molecule (as used in Example 3), retaining all 7 TCR glycosylation sites intact, was used as a control. The data shown in FIG. 8 indicate that the monoglycosylated variant (mol020v14) demonstrated a further increase in in vivo half-life relative to the aglycosylated variant (mol020v13). The molecule with all 7 glycosylation sites intact is comparable to the aglycosylated molecule.

The PK analysis for Mol020v14 was repeated at three dose levels as indicated in the table below. The data showed consistent PK parameters were obtained across wide dose range. The terminal t_{1/2} calculated by non-compartmental analysis was 5.5-7.2 days

| | | |
|---|---|---|
| Dose level | Clearance (ml/hr/Kg) | T½ |

| | | | |
|---|---|---|---|
| 0.665 | mg/Kg | 0.357 ± 0.016 | 7.2 d ± 0.7 |
| 0.0665 | mg/Kg | 0.375 ± 0.048 | 7.1 d ± 0.4 |
| 0.00665 | mg/Kg | 0.457 ± 0.025 | 5.5 d ± 1.4 |

### Example 9 Multidomain Fc Fusion Molecules that Bind MAGEA4 Demonstrate Potent and Specific Activity Against Antigen Positive Cancer Cell Lines

Further assessment of potency and specificity of v14 and v13 was carried out using various cancer cell lines. T cells activation was assessed by IFNγ ELISpot assay and cell killing was assessed using the xCELLigence assay.

### ELISpot

Assays were performed using a human IFNγ ELISPOT kit (BD Biosciences) according to the manufacturer's instructions. Briefly, target cells were prepared at a density of 1×10⁶/ml in assay medium and plated at 50,000 cells per well in a volume of 50 μl. PBMCs isolated from fresh donor blood, were used as effector cells. Fusion molecules were titrated down from 10 nM to give final concentrations represented. Samples were detected using AEC chromagen. Spot counting was performed using a CTL analyser with Immunospot software (Cellular Technology Limited). Ec50 values were calculated from the data.

### Xcelligence

Assays were performed using the xCELLigence platform with appropriate 96 well plates for impedance reading (xCELLigence E-plate 96 PET part number 300600900) and carried out according to the manufacturer's instructions. Target cells were plated at the required density and incubated overnight to allow them to adhere. Test molecules were prepared at various concentrations and 50 μl of each was added to the relevant well such that final concentrations were between 100 fM and 10 nM. Effector cells were used at an effector target cell ratio of 10:1 and plated in 50 μl. A control sample without fusion was also prepared along with samples containing either effector cells alone, or target cells alone. The final volume in each well was adjusted to 200 μl using assay medium. The percentage of cytolysis was determined using the normalised Cell Index (impedance measurement). In all cases, assays were performed in triplicate measurements taken every 2 hours over 96 hours. Ec50 calculations were derived from percent cytolysis curves at 72 hours:

Antigen positive (MAGEA4 +ve; HLA-A*02 +ve) cancer cell lines used in this example were:
NCI-H1703 (NSCLC)
NCI-H1755 (NSCLC)
H314 (Head and Neck carcinoma)
SCaBER (Bladder carcinoma)
UM-UC-3 (Bladder carcinoma)

Antigen negative cells lines were negative for either MAGEA4 or HLA-A*02

### Results

The data shown in FIG. 9 demonstrate that T cell activation was observed against all HLA^{+ve}/Ag^{+ve} cell lines from various indications, with Ec50 values in the pM range. Limited responses were observed in antigen negative cells, even at the highest concentrations of test molecule.

The table below shows an average of Ec50 values obtained from three different PBMC donors.

| | IFNγ release - Ec50 (pM) | | | | |
|---|---|---|---|---|---|
| | NCI-H1703 | NCI-H1755 | H314 | SCaBER | UM-UC-3 |
| Mol020v14 (glyc) | 163 | 39 | 94 | 69 | 314 |
| Mol020v13 (aglyc) | 153 | 31 | 97 | 54 | 526 |

Cancer cell killing was observed at concentrations as low as 0.17 pM in the most sensitive cell lines (NCI-H1703 and NCI-H1755) up to 370 pM.

Ec50 values were calculated from the data obtained at 72 h. Data from one T cell donor are shown in the table below. UM-UC-3 cells were not amenable to testing by xCELLigence assay.

| | Killing - Ec50 (pM) | | | |
|---|---|---|---|---|
| | NCI-H1703 | NCI-H1755 | H314 | SCaBER |
| Mol020v14 (glyc) | 0.38 | <0.17 | 23 | 14* |
| Mol020v13 (aglyc) | 0.38 | <0.17 | 26 | 14* |

*Bottom of curve constrained to 0% for EC50 calculation

Overall these data demonstrate that mol020v13 and mol020v14 give rise to a comparable potent and specific T cell response against antigen positive cancer cells, suitable for therapeutic use.

### Example 10—Multidomain Fc Fusion Molecules that Bind MAGEA4 Show Limited Reactivity Against a Panel of Normal Cells Lines

mol020v13 and mol020v14 were further tested for reactivity against a panel of cells derived from high risk normal tissue types, including, cardiac, muscular, excretory, gastro-intestinal, pulmonary, bone, as well as induced pluripotent stem cells (astrocytes, cardiomyocytes, hepatocytes). In each case reactivity was determined using an ELISpot assay to detect IFNγ and granzyme B release at various concentrations of test molecule up to 10 nM. The lowest concentration of test molecule at which IFNγ was detected was recorded.

For both molecules limited loss of specificity was observed at 1.1 nM with broad loss of specificity observed from 3.3 nM. The lowest concentrations that gave rise to reactivity was 0.55 nM and 1.1 nM for monoglycosylated and aglycosylated respectively.

Overall, the differences between the two molecules were minimal and both molecules exhibit an acceptable profile for therapeutic use

### Example 11—Multidomain Fc Fusion Molecules Comprising TCR Variants that Bind to a HLA-A*02 Restricted Peptide from PreProlnsulin (PPI) Fused to a PD-1 Agonist Binding Domain Retain Target Specificity and are Functional In Vitro

### a) Production of Multidomain Binding Molecules

TCRs that bind to the HLA-A*02 restricted peptide ALWGPDPAAA, derived from PreProinsulin, were isolated by panning TCR phage libraries, and the amino acid sequences of the corresponding TCR alpha and beta variable regions determined. The construction and panning of native TCR phage libraries has been described previously (WO2015136072, WO2017046201, WO2017046198). Soluble TCRs were created by fusing the variable regions to truncated versions of the respective alpha and beta chain constant domains, and a non-native interchain disulphide bond was incorporated between constant domain residues as previously described (WO2003020763). One of the soluble TCRs identified, TCR a2b3, was further mutated to remove potential glycosylation sites and to further optimise the sequence for manufacturing processes. Five TCR sequence variants were tested and referred to by Molecule ID below)

A PD-1 agonist antibody V_{HH} domain was fused to the N terminus of the beta chain of the soluble TCR via a short linker to produce TCR PD-1 agonist binding molecules. To extend in vivo half-life, a functionally silent Fc domain was attached to the C terminus of the TCR alpha chain via a truncated hinge region.

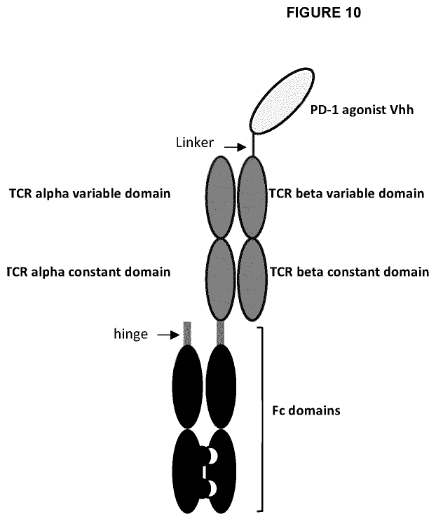

FIG. 10 provides a schematic of the resulting half-life extended TCR PD-1 agonist binding molecule.

### b) Mammalian Expression

Molecules according to part a) were expressed in CHO cells using the Thermo ExpiCHO™ transient expression protocol, followed by purification using immobilized metal affinity chromatography and size exclusion chromatography.

### c) Biophysical CHARACTERISATION

TCR PD-1 agonist binding molecules with an Fc domain were tested for binding to the target peptide and mimetic peptides. Experiments were carried out using single cycle kinetics as described above, except that measurements were performed at 37° C.

TABLE 5

| | | | alpha | beta | | | | | |
|---|---|---|---|---|---|---|---|---|---|
| | TRAV | TRBV | chain | chain | | PPI | | K_{D} | |
| Molecule | SEQ | SEQ | (SEQ | (SEQ | Yield | (ALWGPDPAAA) | | mim1 | Window |
| ID | ID | ID | ID) | ID) | (mg/L) | K_{D} (pM) | T ½ (h) | (nM) | to mim1 |
| a18b16 | 24 | 31 | 36 | 38 | 27 | 39 | 15.6 | 31 | 503 |
| a19b19 | 26 | 34 | 40 | 41 | 39.9 | 57 | 14.0 | 60 | 1052 |
| a19b20 | 26 | 74 | 40 | 80 | 49.5 | 39 | 15.2 | 50 | 1282 |
| a19b21 | 26 | 76 | 40 | 81 | 53.2 | 40 | 15.5 | 65 | 1625 |
| a19b22 | 26 | 78 | 40 | 82 | 43.5 | 40 | 16.7 | 60 | 1500 |

Data show that TCR PD-1 agonist binding molecules including an Fc domain can be produced in mammalian cells at high yield and maintain high affinity recognition of target and a suitable window of binding to mim1. This high level of specificity indicates that the molecules are particularly suitable for therapeutic development as a potential treatment of T1 D.

### d) In Vitro Function—Jurkat NFAT Cell Reporter Assays

To determine the ability of the TCR PD-1 agonist binding molecules to inhibit signalling in activated T cells a NFAT reporter assay was developed. Briefly, Jurkat cells, expressing i) a TCR specific for a HLA-A*02 restricted peptide from Melan A (ELAGIGILTV), ii) PD-1, and iii) a luciferase reporter driven by an NFAT-response element, were incubated with PPI positive beta cell line ECN90 pulsed with a peptide derived from Melan-A to trigger TCR signalling and NFAT promoter-mediated luminescence. Control experiments were performed using PPI negative target cells lines (Mel624, NCI-H1703) in place of ECN90.

Target cells were harvested and plated at 50000 cells/well in OptiP3 media, into the inner 60 wells of a white 96-well cell culture plate that had been pre-coated with β-coat (Univercell Biosolutions). After incubating at 37° C., 5% CO₂ for 16-20 hours, media was removed and assay buffer containing Melan-A peptide was added. No peptide was added to Mel624 melanoma line that naturally present the melan-A peptide. After pulsing for 2 hours at 37° C., 5% CO₂, assay buffer alone or assay buffer containing titrations of TCR PD-1 agonist binding molecules was added to each well. The assay was initiated by immediately adding 50000 Jurkat NFL Mel5 PD-1 effector cells and incubating for 16-20 hours at 37° C., 5% CO₂. Bioluminescent signal was detected and quantified using Bio-Glo™ Luciferase Assay System (Promega) and a luminometer (CLARIOstar). NFAT activity was normalised against TCR-stimulated controls and dose response data was analyzed in Prism (GraphPad) using a four parameter, non-linear least squares fit to determine IC₅₀ values.

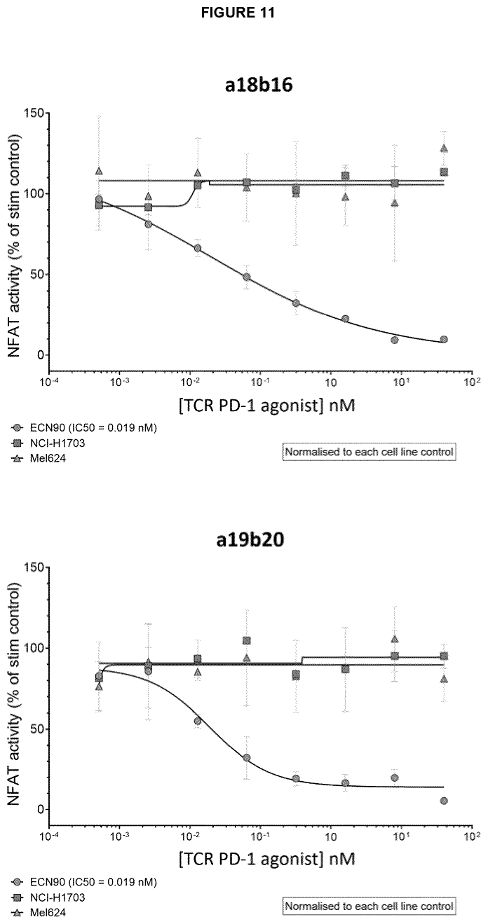

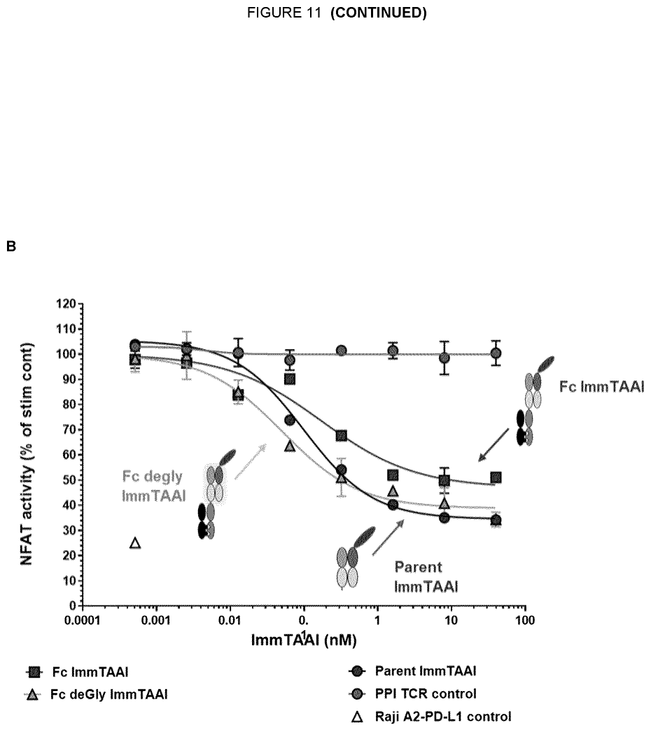

The resulting IC₅₀ values are provided in the table below for each of the indicated TCR-PD1 agonist binding molecules. Values are based on averages from 2 independent experiments. FIG. 11 shows data for 1 experiment from two of the molecules tested.

TABLE 6

| Molecule ID | IC₅₀ (pM) | Max NFAT inhibition (%) |
|---|---|---|
| a18b16 | 24 (n = 2: 19/29) | 89% (90.25 / 86.7) |
| a19b19 | 23 (n = 2: 21/25) | 92% (89.08 / 85) |
| a19b20 | 23 (n = 2: 19/27) | 92% (94.62 / 88.9) |
| a19b21 | 21 (n = 2: 13/29) | 92% (96.64 / 86.56) |
| a19b22 | 112.5 (n = 2: 113/112) | 88% (88.19 / 87.8) |

A similar NFAT based reporter assay was used to assess the impact of the Fc domain on IC₅₀ values.

In this case HLA-A*02 human B lymphoblastoid cells (Raji) pulsed for 2 hours at 37° C., 5% CO₂ with 20 μM PPI peptide were used as target cells. Cells were harvested and plated at 50000 cells/well in assay media (R10 without antibiotics), into the inner 60 wells of a white 96-well cell culture plate. Subsequently, the cells were treated with 2 μg/ml SEB (Staphylococcal enterotoxin B) for 1 hour at 37° C., 5% CO₂. The assay buffer alone or assay buffer containing titrations of TCR PD-1 agonist binding molecules was added to each well. The assay was initiated by immediately adding 50000 Jurkat NFL Mel5 PD-1 effector cells and incubating for 16-20 hours at 37° C., 5% CO₂.

Bioluminescent signal was detected and as described above.

Data were obtained using TCR-PD1 agonist binding molecule a18b16 with an Fc domain as described above and a glycosylated variant with or without the Fc domain.

The data in FIG. 11b show that inclusion of the Fc domain has a minimal impact on in vitro potency.

The data from the reporter assays demonstrate that TCR PD-1 agonist binding molecules can potently inhibit activation of T cells and indicate the therapeutic potential of the molecules for the treatment of T1 D.

### Example 12—TCR-PD1 Agonist Binding Molecules Provide Extended In Vivo Half Life

Pharmacokinetic properties of the TCR PD-1 agonist binding molecule a18b16 with an Fc domain (as described in example 11) was assessed in SCID mice. Test article was dosed intravenously (IV) or subcutaneously (SC) at 1 mg/Kg, with serial sampling of blood over a 21 day period. Four mice were sampled per time point per dosing route., The binding molecule was detected in serum using a bifunctional MSD (Meso Scale Diagnostics) assay. PK parameters were extracted by non-compartmental analysis.

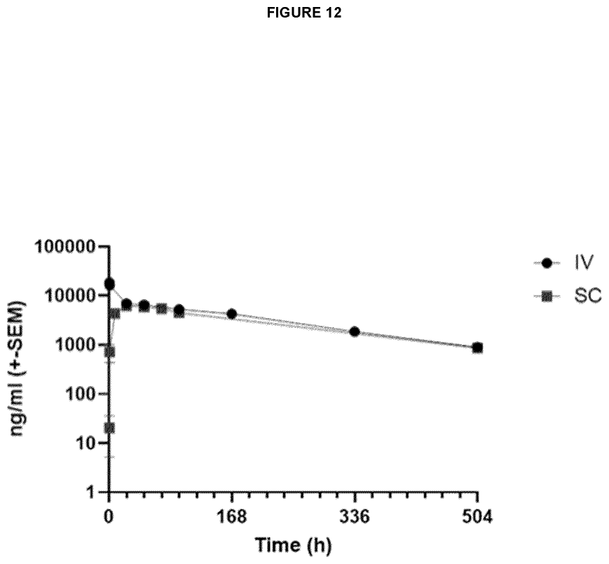

Mean PK parameters are shown in the table below. FIG. 12 shows concentrations of reagent in serum over the course of three weeks.

TABLE 7

| Dose | Tₘₐₓ | Cₘₐₓ | AUCₗₐₛₜ | AUC_{inf} | t_{1/2} | CL ^{a} | V ^{b} |
|---|---|---|---|---|---|---|---|
| Route (N) | (h) | (ng/mL) | (h*ng/mL) | (h*ng/mL) | (h) | (mL/h/kg) | (mL/kg) |
| Intravenous | 0.08 | 18500 | 1760000 | 1970000 | 6.57 | 0.509 | 116 |
| Subcutaneous | 24 | 6210 | 1400000 | 1610000 | 6.89 | 0.621 | 148 |

This study indicated that the TCR PD-1 agonist with Fc has a terminal t_{1/2} of approximately 7 days and a subcutaneous bioavailability of >80%. These properties indicate a therapeutic potential to provide a convenient dosing schedule for the treatment of T1 D.

### Example 13—TCR-PD-1 Agonist Binding Molecules Demonstrate Robust Efficacy in In Vitro Models

### a) Primary Human T Cell IL-2 Assay

TCR PD-1 agonist binding molecules as described in Example 11 were tested to determine their ability to inhibit activation of primary human CD4+ T cells by antigen-presenting cells (APCs). Free PD-1 agonist was used as a control, along with a non-targeted TCR PD-1 agonist control that does not bind to PPI peptide.

Raji cells transduced with an HLA-A*02 β2-microgobulin were used as APCs (Raji-A2). Primary human CD4+ T cells were isolated from PBMCs using a pan T cell isolation kit (Miltenyi). T cells were pre-activated by incubating with irradiated Raji A2 cells, pre-loaded with 1 μg/ml SEB (Sigma). After pre-activation, the expanded T cells were predominantly CD4+ T cells and typically 60-70% PD-1 positive Raji A2 cells were pulsed, or not, with 20 μM PPI peptide at 2×10⁶ cell/ml in R10 for 2 hours at 37° C., 5% CO₂. Raji A2 cells were then loaded with 31.6 ng/ml SEB for 1 hour at 37° C., 5% CO₂ and irradiated with 33Gy. Raji A2 cells were plated at 100,000 cells/well and test molecule added. After 1 hour preincubation, washed pre-activated T cells were added to the Raji A2 cells at 100,000 cells/well and incubated for 48 hours at 37° C., 5% CO₂. Supernatants were collected and IL-2 levels were measured by ELISA (IL2 Ready-SET-Go! ELISA, Invitrogen). IL-2 release was normalised against SEB-stimulated controls and dose response data was analyzed in Prism (GraphPad) using a four parameter, non-linear least squares fit to determine IC₅₀ values.

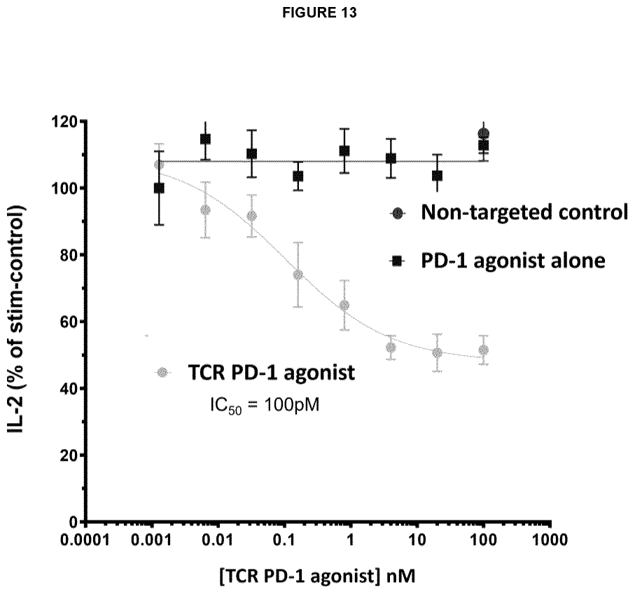

Results demonstrated that in the presence of PPI peptide-pulsed APCs, TCR PD-1 agonist molecules reduced IL-2 production from activated T cells by 40-50%, when present at picomolar levels (FIG. 13). In addition, PD-1 agonist alone, and the non-targeted TCR PD-1 agonist control, did not show a reduction in IL-2 levels, indicating that targeting of the PD-1 agonist to the immune synapse is required for functional activity.

These data demonstrate that targeted TCR PD-1 agonist molecules are potent inhibitors of primary CD4+ T cells. Furthermore, the lack of activity seen with non-targeted molecules indicate the potential to avoid the risk of systemic activation in vivo.

b) Protection of Pancreatic R-Cell Co-Cultured with Autoreactive T Cells

TCR PD-1 agonist binding molecules as described in Example 11 were tested to determine their ability to inhibit killing of the pancreatic b-cell line EndoCbH2-A2 and cytokine release by autoreactive CD8+ T cells.

EndoC-bH2 target cells labelled with mKate 2 (EndoC-bH2 Red) were generated by transducing EndoC-bH2 cells with HLA-A2 β2-microglobulin lentivirus construct and NucLight red lentivirus reagent (Sartorius). Target cells were plated at 5×10⁴ cells per well of a 96 well plate in Optib3 media, incubated over night at 37° C. 5% CO₂. TCR PD-1 agonist molecules, or control molecules, were added at different concentrations and incubated for 2 hours. To initiate the assay, one of two β-cell specific CD8+ T cell clones, having high or low affinity for target cells, was added to EndoC-bH2 red target cells at 5×10⁴ cells per well. PD-L1 transduced EndoC-bH2 red target cells +/− anti-PD-L1 blocking antibody were used as additional controls. Cell killing was determined by quantification of EndoC-bH2 red cell number over time using the IncuCyte S3 imaging system (Sartorius). The number of red nucleus-labelled cells at each time point was normalised to the initial number of objects to take in account variation in cell density in the area visualised. The number of events were acquired in four images and averaged. Cytokine release was measured by V-PLEX Plus Proinflammatory Panel 1 (human) kit in accordance with the manufacturer's instructions (MSD, Meso Scale Diagnostics) using culture supernatants from the IncuCyte killing assays at 24 hours after time point. For the cytokine assay non-stimulated T cells alone were assessed as additional controls. Cytokine release was normalised against stimulated controls and dose response data was analyzed in Prism (GraphPad) using a four parameter, non-linear least squares fit to determine IC₅₀ values.

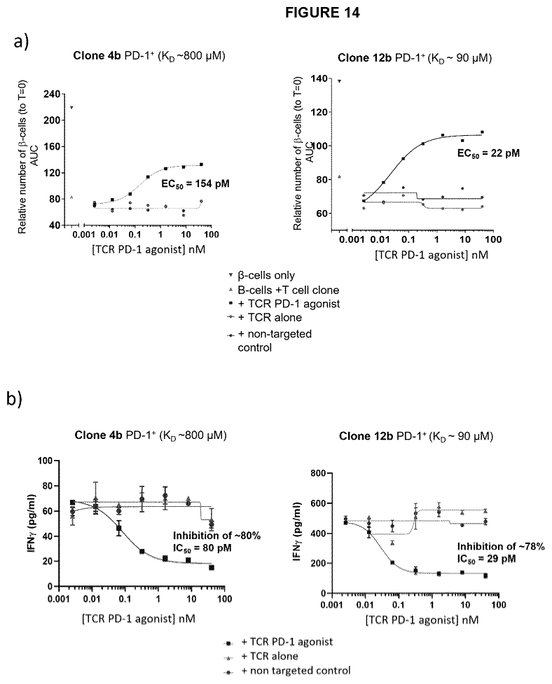

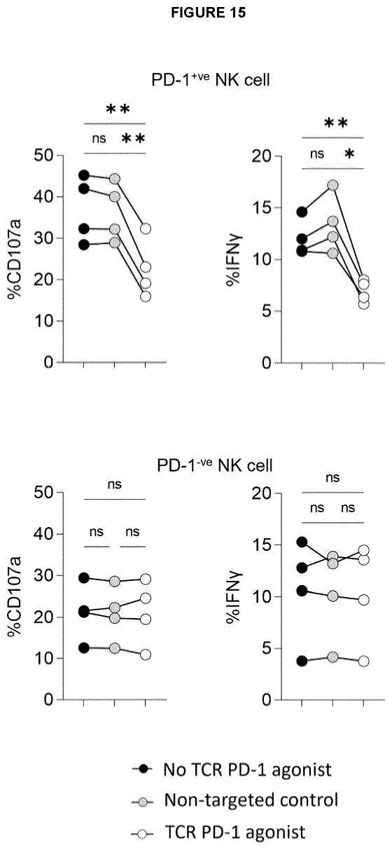

Data showed that the relative number of β cells, when co-cultured in the presence of autoreactive T cells, increased in a dose dependent manner with increasing concentrations of TCR PD-1 agonist binding molecule, thereby demonstrating that the molecules can protect p cells from killing by autoreactive T cells. No effect was observed with the PPI TCR alone or with a non-targeted control (FIG. 14a).

Cell culture supernatants from both co-culture assays were assessed for cytokine production and showed that TCR PD-1 agonist potently inhibits IFNγ production by autoreactive T cells (FIG. 14b).

These data demonstrate that TCR PD-1 agonist binding molecules inhibit killing and cytokine release by T cells that bookend the anticipated affinity range of the natural repertoire of autoreactive T cells, indicating the therapeutic potential of the molecule.

### c) Suppression of PD-1^{+ve} NK Cell Stimulation

TCR PD-1 agonist binding molecules as described in Example 11 were further investigated to determine their ability to inhibit stimulation of PD-1^{+ve} NK cells. To explore if TCR PD-1 agonist can specifically inhibit PD-1+NK cells, NK cells were activated with the pancreatic β cell line EndoC-βH2. Activation was monitored by expression of cytotoxicity marker CD107a and IFNγ production.

Primary human NK cells were isolated from PBMCs using a NK cell isolation kit (Milteny Biotec 130-092-657). The NK cells were incubated 6 days in R10 medium (RPMI-1640 supplemented with 10% heat-inactivated FBS, 2 mM L-glutamine, 1 mM sodium pyruvate) with Dexamethasone (500 ng/mL, Merck, D2915), IL-12 (10 ng/mL, Miltenyi Biotec 130-096-704) IL-15 (25 ng/mL, Peprotech) and IL-18 (100 ng/mL, R&D systems, 9124-IL-050). After 6 days, NK cells were washed in R10 and incubated with or without the TCR-PD1 agonist binding molecule for 4 h (37C, 5% CO₂) with EndoC-βH2 HLA-A2+ cell at ratio (effector/target) ¼ in R10 with monensin, brefeldin A (GolgiPlug and GolgiStop BD) and Anti-CD107a antibody. After activation, NK cells were subject to surface staining (Anti-CD56, Anti-CD3, Anti-PD1 and dead cell marker) for 30 min, then fixed and permeabilized (eBioscience Foxp3 Transcription Factor Staining Buffer Set Cat: 00-5523-00) for IFNγ intra cellular staining.

Data showed that in presence of TCR PD-1 agonist the level CD107 and IFNγ expression decreased in the PD-1 +ve NK cells. No effect was observed on PD-1 −ve NK cells. Thus, TCR PD-1 agonist specifically decreases PD-1+NK cell activation (FIG. 15). Data shown were obtained from 2 independent experiments.

These data demonstrate that TCR PD-1 agonist binding molecules inhibit stimulation of PD-1^{+Ve} NK cells. This provides a potential additional therapeutic mechanism of action and could provide differentiation from other approaches.

### d) Targeting of Beta Cells in Pancreas Tissue Slices

To evaluate the activity of the TCR-PD-1 agonist molecules in a physiologically relevant setting, live pancreas tissue slices from non-diabetic and diabetic tissue donors were treated with TCR PD-1 agonist pre-labelled with a fluorescent dye. Confocal images were captured showing specific targeting of the molecule to b-cells within the islets in both non-diabetic and diabetic donors. Furthermore, an increase of T cell mobility within the islets was observed, suggesting a reduction of b-cell-T cell interaction.

In total, these data obtained across various disease relevant models, evidence the therapeutic potential of the TCR-PD-1 agonist molecules for the treatment of T1 D.

1. A multi-domain binding molecule comprising:
(i) a peptide-major histocompatibility complex (pMHC) binding domain comprising a first variable region linked to a constant region (VC1) and a second variable region linked to a constant region (VC2), wherein VC1 and VC2 dimerise to form the pMHC binding domain;
(ii) an immune cell engaging (ICE) domain; and
(iii) a half-life extending domain comprising a first portion of an IgG Fc region (FC1) and a second portion of an IgG1 Fc region (FC2);
wherein the multi-domain binding molecule comprises:
i) a first polypeptide chain in which the ICE domain is linked to the N terminus of VC1;
ii) a second polypeptide chain in which VC2 is linked via its C terminus to the N terminus of FC1; and
iii) a third polypeptide chain comprising FC2; and
wherein the pMHC binding domain and the ICE domain are capable of binding to a pMHC complex and an immune cell respectively.

2. The multi-domain binding molecule of claim 1, wherein the immune cell engaging domain comprises:
i) an antibody light chain variable domain (ICE-VL) linked to an antibody heavy chain variable domain (ICE-VH); or
ii) a single domain antibody (ICE-SD).

3. The multi-domain binding molecule of claim 1, wherein the immune cell is a T cell and the immune cell engaging domain is selected from:
i) a T cell engaging immune effector domain; or
ii) a T cell engaging an immune suppressor.

4. A multi-domain binding molecule comprising:
(i) a peptide-major histocompatibility complex (pMHC) binding domain comprising a first variable region linked to a constant region (VC1) and a second variable region linked to a constant region (VC2), wherein VC1 and VC2 dimerise to form the pMHC binding domain;
(ii) a T cell engaging immune effector domain comprising an antibody light chain variable domain (TCE-VL) linked to an antibody heavy chain variable domain (TCE-VH); and
(iii) a half-life extending domain comprising a first portion of an IgG Fc region (FC1) and a second portion of an IgG1 Fc region (FC2);
wherein the multi-domain binding molecule comprises:
i) a first polypeptide chain in which the T cell engaging immune effector domain is linked to the N terminus of VC1;
ii) a second polypeptide chain in which VC2 is linked via its C terminus to the N terminus of FC1; and
iii) a third polypeptide chain comprising FC2; and
wherein the pMHC binding domain and the T cell engaging immune effector domain are capable of binding to a pMHC complex and a T cell respectively.

5. The multi-domain binding molecule of claim 4, wherein the T cell engaging immune effector is an ScFv.

6. The multi-domain binding molecule of claim 4 or claim 5, wherein:
(i) VC1 comprises either (a) a TCR alpha (TCRα) variable and constant region; or (b) a TCR beta (TCRβ) variable and constant region; and
(ii) VC2 comprises the other of (a) and (b).

7. The multi-domain binding molecule of claim 6, wherein VC1 comprises the TCRβ variable and constant region and VC2 comprises the TCRα variable and constant region.

8. The multi-domain binding molecule of any one of claims 4-7, wherein the TCE-VL region is linked via its C terminus to the N terminus of the TCE-VH region and the TCE-VH region is linked via its C terminus to the N terminus of VC1.

9. The multi-domain binding molecule of any one of claims 1-8, wherein the immune cell is a T cell and the ICE domain is a CD3 effector domain that activates a T cell through interaction with CD3 and/or a TCR/CD3 complex.

10. The multi-domain binding molecule of any one of claims 1-9, wherein the pMHC binding domain binds to a tumour associated antigen peptide in complex with an MHC.

11. The multi-domain binding molecule of claim 10, wherein the tumour associated antigen is MAGEA4.

12. The multi-domain binding molecule of claim 11, wherein the pMHC binding domain binds to a GVYDGREHTV (SEQ ID NO: 1) HLA-A*02 complex.

13. The multi-domain binding molecule of any one of claims 1-12, wherein two or more of the domains are linked via linkers and/or IgG hinge sequences.

14. The multi-domain binding molecule of claim 13, wherein the linker or linkers have a sequence selected from the group of GGGGS (SEQ ID NO: 34), GGGSG (SEQ ID NO: 35), GGSGG (SEQ ID NO: 36), GSGGG (SEQ ID NO: 37), GSGGGP (SEQ ID NO: 38), GGEPS (SEQ ID NO: 39), GGEGGGP (SEQ ID NO: 40), GGEGGGSEGGGS (SEQ ID NO: 41), GGGSGGGG (SEQ ID NO: 42), GGGGSGGGGSGGGGSGGGGSGGGS (SEQ ID NO: 46), GGGGSGGGGSGGGGSGGGGS (SEQ ID NO: 43), EAAAK (SEQ ID NO: 44) and EAAAKEAAAKEAAAK (SEQ ID NO: 45).

15. The multi-domain binding molecule of any one of claims 1-14, wherein the half-life extending domain comprises one or more amino acid substitutions which facilitate dimerisation of FC1 and FC2.

16. The multi-domain binding molecule of claim 15, wherein:
(i) one of FC1 or FC2 comprises one or more amino acid substitutions selected from the group consisting of T366S, L368A, and Y407V (numbered according to the EU numbering scheme); and
(ii) the other of FC1 or FC2 comprises the amino acid substitution T366W (numbered according to the EU numbering scheme).

17. The multi-domain binding molecule of any one of claims 1-16, wherein FC1 and/or FC2 comprise one or more amino acid substitutions which prevent or reduce binding to FcγR.

18. The multi-domain binding molecule of claim 17, wherein FC1 and/or FC2 comprise a N297G amino acid substitution (numbered according to the EU numbering scheme).

19. The multi-domain binding molecule of any one of claims 1-18, wherein the half-life extending domain comprises one or more amino acid substitutions which promote binding to FcRn.

20. The multi-domain binding molecule of claim 19, wherein FC1 and/or FC2 comprise M252Y/S254T/T256E amino acid substitutions (numbered according to the EU numbering scheme).

21. The multi-domain binding molecule of any one of claims 1-20, wherein the VC1 and/or VC2 comprises one or more amino acid substitutions which remove a glycosylation site.

22. The multi-domain binding molecule of claim 21 wherein the substitution is an N to Q substitution.

23. The multi-domain binding molecule of claim 21 or 22, wherein VC1 comprises either (a) a TCRα variable and constant region or (b) a TCRβ variable and constant region and VC2 comprises the other of (a) and (b), and wherein:
(i) the TCRα chain comprises one or more amino acid substitutions at positions selected from the group consisting of N18, N24, N146, N180 and N191 (numbered according to SEQ ID NO: 25); and/or
(ii) the TCRβ chain comprises an amino acid substitution at position N84 and N186 (numbered according to SEQ ID NO: 27).

24. The multi-domain binding molecule of any preceding claim, wherein the immune cell engaging domain is a T cell engaging immune effector domain, which comprises:
(i) a TCE-VL region comprising CDRs of SEQ ID No: 68, 69, and 70 as CDR1, CDR2 and CDR3 respectively; and
(ii) a TCE-VH region comprising CDRs of SEQ ID No: 71, 72, and 73 as CDR1, CDR2 and CDR3 respectively.

25. The multi-domain binding molecule of any preceding claim, wherein the immune cell engaging domain is a T cell engaging immune effector domain which comprises a TCE-VL at least 80% identical to the sequence of SEQ ID No: 66 and a TCE-VH at least 80% identical to the sequence of SEQ ID No: 67.

26. The multi-domain binding molecule of any preceding claim, wherein VC1 comprises either (a) a TCRα variable and constant region or (b) a TCRβ variable and constant region and VC2 comprises the other of (a) and (b), and wherein:
(i) the TCRα variable domain comprising CDRs of SEQ ID No: 2, 3, and 4 as CDR1, CDR2 and CDR3 respectively; and
(ii) the TCRβ variable domain comprising CDRs of SEQ ID No: 8, 9, and 10 as CDR1, CDR2 and CDR3 respectively.

27. The multi-domain binding molecule of any preceding claim, wherein one of VC1 or VC2 comprises a TCRα variable region at least 80% identical to the sequence of SEQ ID No: 6 and the other of VC1 or VC1 comprises a TCRβ variable region at least 80% identical to the sequence of SEQ ID No: 11.

28. The multi-domain binding molecule of any preceding claim, wherein one of VC1 or VC2 comprises a TCRα constant region that is at least 80% identical to the sequence of SEQ ID No: 29 and the other of VC1 or VC2 comprises a TCRβ constant region that is at least 80% identical to the sequence of SEQ ID No: 31.

29. The multi-domain binding molecule of any preceding claim, wherein one of FC1 or FC2 has an amino acid sequence that is at least 80% identical to the sequence of SEQ ID No: 49 and the other of FC1 or FC2 has an amino acid sequence that is at least 80% identical to the sequence of SEQ ID No: 50.

30. The multi-domain binding molecule of any preceding claim, wherein:
(i) the first polypeptide chain of the molecule has a sequence at least 80% identical to the sequence of SEQ ID NO: 56;
(ii) the second polypeptide chain of the molecule has a sequence at least 80% identical to the sequence of SEQ ID NO: 54.
(iii) the third polypeptide chain of the molecule has a sequence at least 80% identical to the sequence of SEQ ID NO: 52.

31. The multi-domain binding molecule of any preceding claim, wherein the multi-domain binding molecule comprises:
(i) a first polypeptide chain comprising in the following order, from N-terminus to C-terminus
a. an amino acid sequence of an anti-CD3 scFv,
b. optionally followed by a linker sequence provided in SEQ ID NO: 34, and
c. an amino acid sequence of a TCRβ variable and constant region;
(ii) a second polypeptide chain comprising in the following order, from N-terminus to C-terminus,
a. an amino acid sequence of a TCRα variable and constant region,
b. optionally followed by a truncated hIgG1 hinge sequence provided in SEQ ID NO: 33 and
c. an Fc region having the sequence provided in SEQ ID NO: 50;
(iii) a third polypeptide chain comprising an Fc region having the sequence provided in SEQ ID NO: 52;
wherein the TCRα chain and TCRβ chain dimerise to form a peptide-major histocompatibility complex (pMHC) binding domain.

32. A nucleic acid encoding the multi-domain binding molecule of any one of claims 1-31.

33. An expression vector comprising the nucleic acid of claim 32.

34. A host cell comprising the nucleic acid of claim 32 or the expression vector of claim 33.

35. A method of making the multi-domain binding molecule of any one of claims 1-31, comprising maintaining the host cell of claim 34 under optimal conditions for expression of the nucleic acid of claim 32 or the expression vector of claim 33 and isolating the multi-domain antigen binding polypeptide.

36. A pharmaceutical composition comprising the multi-domain binding molecule of any one of claims 1-31, the nucleic acid of claim 32, the expression vector of claim 33 or the host cell of claim 34.

37. The multi-domain binding molecule of any one of claims 1-31, the nucleic acid of claim 32, the expression vector of claim 33, the host cell of claim 34 or the pharmaceutical composition of claim 36, for use as a medicament.

38. A method of treatment comprising administering the multi-domain binding molecule of any one of claims 1-31, the nucleic acid of claim 32, the expression vector of claim 33, the host cell of claim 34 or the pharmaceutical composition of claim 32 to a patient in need thereof.

39. The multi-domain binding molecule of any one of claims 1-31, the nucleic acid of claim 32, the expression vector of claim 33, the host cell of claim 34 or the pharmaceutical composition of claim 36 for use in treating cancer.

40. The multi-domain binding molecule, nucleic acid, expression vector, host cell or pharmaceutical composition of claim 39, wherein the cancer expresses MAGEA4.

41. The multi-domain binding molecule of any one of claims 1-31, the nucleic acid of claim 32, the expression vector of claim 33, the host cell of claim 34 or the pharmaceutical composition of claim 36 for use in treating an infectious disease or an autoimmune disease.
